(19) United States
(12) Patent Application Publication
(10) Pub. No.: US 2024/0254509 A1
(43) Pub. Date: Aug. 1, 2024

(54) PRODUCTS AND METHODS FOR TREATMENT OF DYSTROPHIN-BASED MYOPATHIES USING CRISPR-CAS9 TO CORRECT DMD EXON DUPLICATIONS

(71) Applicant: RESEARCH INSTITUTE AT NATIONWIDE CHILDREN'S HOSPITAL, Columbus, OH (US)

(72) Inventors: Kevin Flanigan, Columbus, OH (US); Anthony Aaron Stephenson, Columbus, OH (US)

(21) Appl. No.: 18/279,169

(22) PCT Filed: Mar. 4, 2022
(86) PCT No.: PCT/US22/18829

§ 371 Date: Aug. 28, 2023

Related U.S. Application Data
(60) Provisional application No. 63/156,443, filed on Mar. 4, 2021.

(51) Int. Cl. C12N 15/86; A61P 21/00; C12N 9/22; C12N 15/113

(52) U.S. Cl. CPC C12N 15/86; A61P 21/00; C12N 9/22; C12N 15/113; C12N 2310/20; C12N 2750/14143; C12N 2800/80; C12N 2830/008

(57) ABSTRACT

The disclosure relates to the field of gene therapy for the treatment of a muscular dystrophy including, but not limited to, Duchenne's muscular dystrophy (DMD), Becker's muscular dystrophy (BMD), or intermediate muscular dystrophy (IMD). More particularly, the disclosure provides nucleic acids, including nucleic acids comprising guide RNAs (gRNAs) and nucleic acids encoding gRNAs to be used with nucleic acids encoding clustered regularly-interspaced short palindromic repeat associated protein 9 (Cas9), and adeno-associated virus (AAV) comprising the nucleic acids to deliver nucleic acids encoding guide RNAs and Cas9 to correct single or multiple DMD exon duplication mutations for use in treating a muscular dystrophy including, but not limited to, DMD, BMD, or IMD, resulting from an exon duplication mutation amenable to CRISPR-Cas9 therapy of the DMD gene.

### STATEMENT OF GOVERNMENT INTEREST

This invention was made with government support under grant no. AR070604 from the National Institutes of Health. The government has certain rights in the invention.

### INCORPORATION BY REFERENCE OF THE SEQUENCE LISTING

This application contains, as a separate part of disclosure, a Sequence Listing in computer-readable form (filename: 55009_Seqlisting.txt; Size: 57,093 bytes: Created: Mar. 2, 2022) which is incorporated by reference herein in its entirety.

### FIELD

This disclosure relates to the field of gene therapy for the treatment of muscular dystrophy. More particularly, the disclosure provides products, methods, and uses for a ne gene therapy for treating, ameliorating, delaying the progression of, and/or preventing a muscular dystrophy involving CRIPSR/Cas9 gene editing for correction of DMD exon duplications.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often lead to divorce, especially if responsibility for the muscular dystrophy can be laid at the feet of one of the parental partners. The burden of quest to find a cure often becomes a life-long, highly focused effort that detracts and challenges every aspect of life. Beyond the family, the the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). It is the most common severe childhood form of muscular dystrophy affecting 1 in 5000 newborn males. DMD is caused by mutations in the DMD gene leading to absence of dystrophin protein (427 KDa) in skeletal and cardiac muscles, as well as the gastrointestinal tract and retina. Dystrophin not only protects the sarcolemma from eccentric contractions, but also anchors a number of signaling proteins in close proximity to sarcolemma. Another form of MD is Becker Muscular Dystrophy (BMD). BMD, like DMD, is a genetic disorder that gradually makes the body's muscles weaker and smaller. BMD affects the muscles of the hips, pelvis, thighs, and shoulders, as well as the heart, but is known to cause less severe problems than DMD.

Many clinical cases of DMD are linked to deletion mutations in the DMD gene. In contrast to the deletion mutations, DMD exon duplications account for around 5% of disease-causing mutations in unbiased samples of dystrophinopathy patients [Dent et al., Am J Med Genet, 134(3): 295-298 (2005)], although in some catalogues of mutations the number of duplications is higher, including that published by the United Dystrophinopathy Project by Flanigan et al. [Hum Mutat, 30(12): 1657-1666 (2009)], in which it was 11%.

Other forms of MD are Becker Muscular Dystrophy (BMD) and intermediate muscular dystrophy (IMD). BMD is one of nine types of muscular dystrophies, a group of genetic, degenerative diseases primarily affecting voluntary muscles. BMD is also caused by a change in the dystrophin gene, which makes the protein too short. The flawed dystrophin puts muscle cells at risk for damage with normal use. See also, U.S. Patent Application Publication Nos. 2012/0077860, published Mar. 29, 2012; 2013/0072541, published Mar. 21, 2013; and 2013/0045538, published Feb. 21, 2013. IMD is a categorization of muscular dystrophy phenotype for patients who walk past the age of 12 but stop walking by age 15. The use of an IMD classification of patients is helpful to describe patients who are less severe than is typical for DMD but more severe than is typical for BMD.

Despite many lines of research following the identification of the DMD gene, treatment options for various muscular dystrophies involving the DMD gene are limited. Thus, there remains a need in the art for treatments for muscular dystrophies including, but not limited to DMD, BMD, and IMD.

### SUMMARY

The disclosure provides products, methods, and uses for a new gene therapy for treating, ameliorating, delaying the progression of, and/or preventing a muscular dystrophy involving a mutation resulting from a DMD exon duplication. More particularly, the disclosure provides products, methods, and uses for a new gene therapy for treating, ameliorating, delaying the progression of, and/or preventing a muscular dystrophy involving CRIPSR/Cas9 gene editing for correction of DMD exon duplications.

The disclosure provides a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276. In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

The disclosure provides an adeno-associated virus (AAV) comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276. In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1. In some aspects, the AAV lacks rep and cap genes. In some aspects, the AAV is a recombinant AAV (rAAV) or a self-complementary recombinant AAV (scAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, the AAV is AAV1, AAV9 or AAVrh.74.

The disclosure provides a nanoparticle, extracellular vesicle, or exosome comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276. In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

The disclosure provides a composition comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; and a pharmaceutically acceptable carrier. In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

The disclosure provides a composition comprising an AAV comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; and a pharmaceutically acceptable carrier. In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1. In some aspects, the AAV lacks rep and cap genes. In some aspects, the AAV is a recombinant AAV (rAAV) or a self-complementary recombinant AAV (scAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, the AAV is AAV1, AAV9 or AAVrh.74.

The disclosure provides a composition comprising a nanoparticle, extracellular vesicle, or exosome comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; and a pharmaceutically acceptable carrier.

The disclosure provides a method of correcting a mutation of the dystrophin (DMD) gene in a cell comprising contacting the cell with
(a) (i) a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (ii) an AAV comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (iii) a nanoparticle, extracellular vesicle, or exosome comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; or
   (iv) a composition comprising the aforesaid nucleic acid; the aforesaid AAV; or the aforesaid nanoparticle, extracellular vesicle, or exosome; and a pharmaceutically acceptable carrier; and
(b) (i) a nucleic acid encoding a Cas9 enzyme or a functional fragment thereof;
   (ii) an AAV comprising a nucleic acid encoding a Cas9 enzyme or a functional fragment thereof;
   (iii) a nanoparticle, extracellular vesicle, or exosome comprising a nucleic acid encoding a Cas9 enzyme or a functional fragment thereof; or
   (iv) a composition comprising the nucleic acid encoding a Cas9 enzyme or a functional fragment thereof, the AAV comprising a nucleic acid encoding a Cas9 enzyme or a functional fragment thereof, or the nanoparticle, extracellular vesicle, or exosome comprising a nucleic acid encoding a Cas9 enzyme or a functional fragment thereof.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1. In some aspects, the AAV lacks rep and cap genes. In some aspects, the AAV is a recombinant AAV (rAAV) or a self-complementary recombinant AAV (scAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, the AAV is AAV1, AAV9 or AAVrh.74.

In some aspects, the nucleic acid encoding the Cas9 enzyme or the functional fragment thereof comprises at least or about 70% identity to the nucleotide sequence set forth in SEQ ID NO: 277 or 278.

The disclosure provides a method of treating, ameliorating, and/or preventing a muscular dystrophy in a subject having a mutation in the dystrophin (DMD) gene comprising administering to the subject an effective amount of
(a) (i) a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (ii) an AAV comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (iii) a nanoparticle, extracellular vesicle, or exosome comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; or
   (iv) a composition comprising the aforesaid nucleic acid; the aforesaid AAV; or the aforesaid nanoparticle, extracellular vesicle, or exosome; and a pharmaceutically acceptable carrier; and
(b) (i) a nucleic acid encoding a Cas9 enzyme or a functional fragment thereof;
   (ii) an AAV comprising a nucleic acid encoding a Cas9 enzyme or a functional fragment thereof;
   (iii) a nanoparticle, extracellular vesicle, or exosome comprising a nucleic acid encoding a Cas9 enzyme or a functional fragment thereof; or
   (iv) a composition comprising the nucleic acid encoding a Cas9 enzyme or a functional fragment thereof, the AAV comprising a nucleic acid encoding a Cas9 enzyme or a functional fragment thereof, or the nanoparticle, extracellular vesicle, or exosome comprising a nucleic acid encoding a Cas9 enzyme or a functional fragment thereof.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1. In some aspects, the AAV lacks rep and cap genes. In some aspects, the AAV is a recombinant AAV (rAAV) or a self-complementary recombinant AAV (scAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, the AAV is AAV1, AAV9 or AAVrh.74.

In some aspects, the nucleic acid encoding the Cas9 enzyme or the functional fragment thereof comprises at least or about 70% identity to the nucleotide sequence set forth in SEQ ID NO: 277 or 278.

In some aspects, the muscular dystrophy is Duchenne's muscular dystrophy (DMD), Becker's muscular dystrophy (BMD), or intermediate muscular dystrophy (IMD).

In some aspects, the mutation is a single- or multiple-exon duplication of the DMD gene. In some aspects, the single- or multiple-exon duplication is involving surrounding, or affecting exon 2 or 3 of the DMD gene. In some aspects, the duplication is a duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene.

The disclosure provides the use of
(a) (i) a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (ii) an AAV comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276;
   (iii) a nanoparticle, extracellular vesicle, or exosome comprising a nucleic acid comprising a nucleotide sequence encoding an RNA sequence comprising at least 90% identity to the sequence set forth in any one of SEQ ID NOs: 1-184; a nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; a nucleotide sequence comprising the RNA sequence set forth in any one of SEQ ID NOs: 1-184; or a nucleotide sequence that specifically hybridizes to a target nucleic acid of the DMD gene comprising the nucleotide sequence set forth in any one of SEQ ID NOs: 185-276; or
   (iv) a composition comprising the aforesaid nucleic acid; the aforesaid AAV; or the aforesaid nanoparticle, extracellular vesicle, or exosome; and a pharmaceutically acceptable carrier; and
(b) (i) a nucleic acid encoding a Cas9 enzyme or a functional fragment thereof;
   (ii) an AAV comprising a nucleic acid encoding a Cas9 enzyme or a functional fragment thereof;
   (iii) a nanoparticle, extracellular vesicle, or exosome comprising a nucleic acid encoding a Cas9 enzyme or a functional fragment thereof; or
   (iv) a composition comprising the nucleic acid encoding a Cas9 enzyme or a functional fragment thereof, the AAV comprising a nucleic acid encoding a Cas9 enzyme or a functional fragment thereof, or the nanoparticle, extracellular vesicle, or exosome comprising a nucleic acid encoding a Cas9 enzyme or a functional fragment thereof for the preparation of a medicament for expressing the dystrophin (DMD) gene in a cell; for treating, ameliorating, and/or preventing a muscular dystrophy; and/or for the preparation of a medicament for treating, ameliorating, and/or preventing a muscular dystrophy.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1.

In some aspects, the nucleic acid further comprises a promoter sequence. In some aspects, the promoter is any of U6, U7, tRNA, H1, minimal CMV, T7, EF1-alpha, minimal EF1-alpha, or a muscle-specific promoter. In some aspects, the promoter is U6 or H1. In some aspects, the muscle-specific promoter is unc45b, tMCK, minimal MCK, CK6, CK7, CK8, MHCK7, CK8e, SPC5-12, or CK1. In some aspects, the AAV lacks rep and cap genes. In some aspects, the AAV is a recombinant AAV (rAAV) or a self-complementary recombinant AAV (scAAV). In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, the AAV is AAV1, AAV9 or AAVrh.74.

In some aspects, the nucleic acid encoding the Cas9 enzyme or the functional fragment thereof comprises at least or about 70% identity to the nucleotide sequence set forth in SEQ ID NO: 277 or 278.

In some aspects, the muscular dystrophy is Duchenne's muscular dystrophy (DMD), Becker's muscular dystrophy (BMD), or intermediate muscular dystrophy (IMD).

In some aspects, the mutation is a mutation of the DMD gene.

In some aspects, the mutation is a single- or multiple-exon duplication of the DMD gene. In some aspects, the single- or multiple-exon duplication is involving surrounding, or affecting exon 2 or 3 of the DMD gene. In some aspects, the duplication is a duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene.

In some aspects, the methods or uses of the disclosure result in increased expression of dystrophin protein in the cell or in the subject. In some aspects, the methods or uses of the disclosure inhibit progression of dystrophic pathology in the subject. In some aspects, the methods or uses of the disclosure improve muscle function in the subject. In some aspects, the improvement in muscle function is an improvement in muscle strength. In some aspects, the improvement in muscle function is an improvement in stability in standing and walking.

In some aspects, the nucleic acid, AAV, nanoparticle, extracellular vesicle, exosome, or composition, or medicament is formulated for intramuscular injection, oral administration, subcutaneous, intradermal, or transdermal transport, injection into the blood stream, or for aerosol administration.

Other features and advantages of the disclosure will become apparent from the following description of the drawing and the detailed description. It should be understood, however, that the drawing, detailed description, and the specific examples, while indicating embodiments of the disclosed subject matter, are given by way of illustration only, because various changes and modifications within the spirit and scope of the disclosure will become apparent to those skilled in the art from this detailed description.

### BRIEF DESCRIPTION OF THE DRAWINGS

FIG. 1A-B shows a representation of exon duplication correction using a single gRNA to target a duplicated site. FIG. 1A shows a representative single exon duplication and a potential CRISPR-Cas9-mediated corrective therapy. A single gRNA targeted (orange triangles) within the duplicated region will cleave both copies of the duplicated region and catalyze reversion to the normal coding sequence. FIG. 1B shows a representative multi-exon duplication and a potential CRISPR-Cas9-mediated corrective therapy. A single gRNA targeted (orange triangles) within the duplicated region (gray shaded region) will cleave both copies of the duplicated region and catalyze reversion to the normal coding sequence.

FIG. 2 shows a human DMD partial gene map overlaid with Staphylococcus aureus (green) and Campylobacter jejuni (blue) gRNA target sites within intron 1 (white) exon 2 (black) and intron 2 (cyan). Scale bar tick marks represent 100 base pairs (bp).

FIG. 3 shows T7E1 mutation detection assay on PCR amplicons generated using gDNA extracted from HEK293 cells following transient expression of plasmids encoding SaCas9 and gRNA as indicated above each lane. Input amplicon (“-”) was run alongside amplicon treated with T7E1 enzyme (“+”) on a 10% PA-TBE gel and imaged after staining with ethidium bromide. Fragments C-E are arbitrarily named amplicons from different regions of the DMD gene that span one or more of the gRNA target sites. The amplicons were designed to work within the length and position limitations of T7E1 mutation detection assay. The target sites were scattered around the gene and thus no one ˜600 bp amplicon covered all of them for the sake of the T7E1 assay. Thus, amplicons were arbitrarily named to keep track of them for the sake of the mutation detection assay. For each different amplicon, gDNA from untreated HEK293 was used as a negative control (“untreated”). For each reaction, locations of expected cleavage fragments are marked with asterisks (“*”). “10 bp ins control” represents a positive control containing equimolar amounts of two DNAs that differ by a 10 bp insertion. Note the strong evidence of editing using hDSA-001, hDSA-002, hDSA-027, and hDSA-030 gRNAs.

FIG. 4 shows T7E1 mutation detection assay on PCR amplicons generated using gDNA extracted from HEK293 cells following transient expression of plasmids encoding CjCas9 and gRNA as indicated above each lane. Input amplicon (“-”) was run alongside amplicon treated with T7E1 enzyme (“+”) on a 10% PA-TBE gel and imaged after staining with ethidium bromide. Fragments B-E are arbitrarily named amplicons from different regions of the DMD gene that span one or more gRNA target site(s). For each different amplicon, gDNA from untreated HEK293 was used as a negative control (“untreated”). For each reaction, locations of expected cleavage fragments are marked with asterisks (“*”). Note the strong evidence of editing using hDCJ-003, hDCJ-008, hDCJ-027, and hDCJ-031 gRNAs.

FIG. 5 shows human DMD partial gene map overlaid with Staphylococcus aureus gRNA target sites within intron 3. Scale bar tick marks represent 100 bp. For mutations in which the target sequence is duplicated (e.g., duplication of exons 2-6), simultaneous cutting by Cas9 at both sites results in deletion of the intervening duplicated sequence and thus restoration of the normal exon arrangement as in FIG. 1B.

FIG. 6 shows T7E1 mutation detection assay on PCR amplicons generated using gDNA extracted from HEK293 cells following transient expression of plasmids encoding SaCas9 and gRNA as indicated above each lane. Input amplicon (“−”) was run alongside amplicon treated with T7E1 enzyme (“+”) on a 10% PA-TBE gel and imaged after staining with ethidium bromide. Fragments α-γ are arbitrarily named amplicons from different regions of the DMD gene that span one or more gRNA target sites. For each different amplicon, gDNA from untreated HEK293 was used as a negative control (“untreated”). For each reaction, locations of expected cleavage fragments are marked with asterisks (“*”). “10 bp ins control” represents a positive control containing equimolar amounts of two DNAs that differ by a 10 bp insertion. Note the strong evidence of editing using JHI3SA003, JHI3SA007, JHI3SA008, JHI3SA013, JHI3SA015, JHI3SA016, and JHI3SA017 gRNAs.

FIG. 7 shows RT-PCR analysis of DMD exons 1-3 in Dup2 patient cells treated with a 1:1 mixture of rAAV encoding MHCK7 promoter-driven Cas9 and scAAV encoding three copies of gRNA hDSA030 driven by U6 promoters at three doses; high (H), medium (M), and low (L) as indicated in Table 3. Cells were then transdifferentiated into myotubes for two weeks before extraction of whole RNA. After cDNA synthesis from whole RNA, PCR was performed using primers in the DMD 5′ UTR and exon 3, resulting in a band of ˜350 bp for Dup2 as indicated by untreated samples (U) and ˜300 bp for the wild-type sequence as shown by heathy control RNA. A minor band corresponding to complete deletion of exon 2 (del2) is also observed at ˜260 bp. RT neg represents an RT-PCR reaction without RNA and NTC represent a PCR reaction without template cDNA. n=3 biological replicates except for group H where n=2. Bands were quantified via densitometry using ImageJ software and plotted as percent of total amplicon density (percent of total transcripts). Bars represent means and error bars are standard deviations.

FIG. 8 shows RT-PCR analysis of DMD exons 1-8 in Dup2-6 patient cells with a 1:1 mixture of rAAV encoding MHCK7 promoter-driven Cas9 and scAAV encoding three copies of gRNA hDSA030 driven by U6 promoters at three doses; high (H), medium (M), and low (L) as indicated in Table 3. Cells were then transdifferentiated into myotubes for two weeks before extraction of whole RNA. After cDNA synthesis from whole RNA, PCR was performed using primers in the DMD 5′ UTR and exon 8, resulting in a band of ˜1300 bp for Dup2-6 as indicated by untreated samples (U) and ˜700 bp for the wild-type sequence as shown by heathy control RNA. RT neg represents an RT-PCR reaction without RNA and NTC represent a PCR reaction without template cDNA. n=3 biological replicates except for group M where n=2. Bands were quantified via densitometry using ImageJ software and plotted as percent of total amplicon density (percent of total transcripts). Bars represent means and error bars are standard deviations.

FIG. 9 shows a cartoon representation of the AAV-vectorized approach to express Cas9 and gRNA in a Dup2 DMD patient muscle fiber. Upon expression, Cas9 and gRNA bind and translocate to the nucleus where Cas9 is guided to a genomic DNA target site programmed by the gRNA. When targeting within the duplicated region the Cas9 cut site is duplicated which results in simultaneous binding and cleavage by Cas9 at both sites removing the intervening duplicated DNA sequence. After DNA repair to fix the broken DNA backbone, the duplication mutation is removed and normal exon arrangement is restored which leads to expression of dystrophin and restoration of normal muscle fiber physiology.

FIG. 10 shows immunofluorescence microscopy images of representative regions of tibialis anterior muscle cross sections from mice four weeks after intramuscular injection (n=4 biological replicates each) of rAAV encoding MHCK7 promoter-driven Cas9 and scAAV encoding three copies of gRNA mDSA010 driven by U6 promoters at three doses; high (H), medium (M), and low (L) as indicated in Table 4. Cross sections were co-stained with a dystrophin antibody (top panel, red channel) and a laminin antibody (green channel, not shown). Sarcolemmal dystrophin accumulation was quantified for each individual fiber for each whole muscle cross section using an intensity-under-the-mask method with the laminin channel serving to determine the sarcolemma mask coordinates. Fibers with at least 30% of the sarcolemma containing dystrophin staining were counted as positive. A mask was then generated, overlayed on each fiber analyzed, and colored with a continuous rainbow gradient based upon the fiber's percent dystrophin-positive perimeter with 0-0.99% in purple and 100% in red. Percent of fibers with >30% dystrophin-positive sarcolemma perimeter were then graphed with bars representing mean percent of dystrophin positive fibers for each treatment group and error bars representing standard deviations.

FIG. 11A-C shows RT-PCR analysis of DMD transcripts in cells from two patients with a Dup2 mutation (FIG. 11A) and one patient with a Dup2-6 mutation (FIG. 11B) after treatment of the cells with a 1:1 mixture of rAAV encoding MHCK7 promoter-driven Cas9 and scAAV encoding three copies of gRNA hDSA030 driven by U6 promoters at a MOI of 4E6 vg/cell. Cells were then transdifferentiated into myotubes for two weeks before extraction of whole RNA. After cDNA synthesis from whole RNA, PCR was performed using primers in the DMD 5′ UTR and exon 8, resulting in a band of ˜1300 bp for Dup2-6 as indicated by untreated samples (Untreated) and ˜700 bp for the wild-type sequence (WT). n=3 biological replicates. Bands were quantified via densitometry using ImageLab Software and plotted as percent of total amplicon density (percent of total transcripts) (FIG. 11C). Bars represent means and error bars are standard deviations.

### DETAILED DESCRIPTION

The disclosure provides products, methods, and uses for treating, ameliorating, delaying the progression of, and/or preventing a dystrophinopathy or a muscular dystrophy involving the DMD gene. Dystrophinopathies are rare (˜1 in 5,000 live male births) but most often fatal diseases caused by mutations in the DMD gene which codes for dystrophin, the vital, muscle-specific structural protein. Disease severity ranges from muscle weakness later in life for the mildest forms of BMD to complete loss of ambulation in adolescence and death from cardiac and respiratory complications in the teens and early twenties for the most severe forms of DMD. In addition to the devastating impact on patients, the socioeconomical and psychological burden on families is enormous. Most patients have no highly effective therapeutic options and are typically treated with supportive care and corticosteroids that provide only very modest benefits and cause serious side-effects.

More specifically, the disclosure provides products, methods, and uses for treating, ameliorating, delaying the progression of, and/or preventing a muscular dystrophy involving duplication mutations in one or more DMD exons.

The products and methods provided herein provide for the expression of a full-length dystrophin protein, or a functional form of dystrophin protein, for use in treating, ameliorating, or preventing a muscular dystrophy resulting from such duplication mutations affecting various regions of the DMD gene. DMD, the largest known human gene, provides instructions for making a protein called dystrophin. Dystrophin is located primarily in muscles used for movement (skeletal muscles) and in heart (cardiac) muscle. In some aspects, the mutation is a single- or multiple-exon duplication involving, surrounding, or affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), the largest known human gene, which encodes dystrophin. Dystrophin is located primarily in muscles used for movement (skeletal muscles) and in heart (cardiac) muscle.

More particularly, the disclosure provides nucleic acids comprising nucleotide sequences encoding guide RNAs (gRNAs), nucleic acids comprising nucleotide sequences of the guide RNAs (gRNAs), nucleic acids encoding CRISPR-Cas9 enzymes, and/or CRISPR-Cas9 enzymes to be used in a CRISPR-Cas9-based strategy to correct single- or multiple-exon duplication of the DMD gene, vectors comprising the nucleic acids for carrying out the exon duplications in various DMD regions, and methods for treating, ameliorating, delaying the progression of, and/or preventing a muscular dystrophy involving duplication mutations in one or more DMD exons. The disclosure therefore provides products, methods, and uses for restoring full-length dystrophin, or a functional form of dystrophin, to a vast cohort of muscular dystrophy patients with diverse mutations of the DMD gene.

### Guide RNAs and Target Sites

The disclosure includes gRNAs to guide Cas9 to user-chosen DNA sites and target sites on the DMD gene for guide RNA targeting, and Cas9 to generate DNA double-stranded breaks at user-chosen sites or target sites on the DMD gene. As used herein, “target”, “target site”, “target sequence” or “target nucleic acid” is either the forward or reverse strand of the sequences provided herein designated as target sequence. Thus, the target is the coding strand or its complement.

Cas9 requires double-stranded DNA to bind and cut; however, the gRNA anneals to only one of the two strands. Despite this, Cas9 binds and cuts both strands of the given sequences. The natural CRISPR Cas9 system contains two RNAs, one is called the crRNA and contains sequences called spacer (assigns its targeting specificity) direct repeat (helps it bind with tracrRNA and Cas9) and a tracrRNA which contains a region complementary to the crRNA direct repeat and anneals to the crRNA direct repeat sequence such that they form a dsRNA that binds to Cas9. Guide RNAs can target either the coding or non-coding strand. The strand a gRNA should be designed to bind depends on which strand the PAM sequence is on. The strand that contains the PAM (e.g., 5′-NNGRRT-3′ (SEQ ID NO: 279) for SaCas9 and 5′-NNNNRYAC-3′ (SEQ ID NO: 280) for CjCas9) is called the non-target strand and it contains the protospacer sequence which matches the sequence of the corresponding spacer region of the gRNA. The spacer region of the gRNA thus binds to the non-PAM-containing strand (the target strand). The target sequences given in the Table 1 are coding sequences of the DMD gene and thus can be either the target or non-target strand (i.e., sense or antisense). Cas9 requires double-stranded DNA where one strand contains the PAM and the other contains the target sequence (i.e., the target strand).

Table 1 provided herein below provides Staphylococcus aureus and Campylobacter jejuni gRNA sequences that target various regions of the human and or mouse DMD gene, including full gRNA sequences and spacer sequences of the gRNAs, and the target sequences for each of the gRNAs.

Table 2 provided herein below provides exemplary Staphylococcus aureus and Campylobacter jejuni Cas9 coding sequences as used in the methods of the disclosure. The provision of these sequences herein is for exemplary purposes and is not meant to limit the methods of the disclosure to these particular Cas9 sequences. As set out herein above, the methods of the disclosure are meant to be practiced with any Cas9 species, homolog, ortholog, or variant, including functional fragments thereof.

More particularly, the disclosure provides nucleic acids comprising sequences designed to bind to various DMD exon or intron sequences to provide a full-length dystrophin protein, or a functional form of dystrophin, for use in treating a muscular dystrophy resulting from a mutation involving, surrounding, or affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3,3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene. The disclosure provides nucleic acids comprising nucleotide sequences encoding guide RNAs, and vectors, such as recombinant adeno-associated virus (rAAV) and self-complementary adeno-associated virus (scAAV), comprising the nucleic acids to deliver nucleic acids encoding the guide RNA and Cas9 to provide a full-length dystrophin, or a functional form of dystrophin, for use in treating a muscular dystrophy resulting from the mutations involving, surrounding, or affecting exon 2 or 3 including, but not limited, to duplication of exon(s) 2, 2-3,2-4, 2-5, 2-6,2-7, 2-8,2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9,3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene. The disclosure also provides nucleic acids comprising guide RNA (gRNA) nucleotide sequences targeting the DMD gene. These sequences are designed to bind to various DMD exon or intron sequences to provide a full-length dystrophin protein, or a functional form of dystrophin protein, for use in treating a muscular dystrophy resulting from a mutation involving, surrounding, or affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene.

The disclosure includes various nucleic acids comprising, consisting essentially of, or consisting of the various nucleotide sequences described herein. In some aspects, the nucleic acid comprises the nucleotide sequence. In some aspects, the nucleic acid consists essentially of the nucleotide sequence. In some aspects, the nucleic acid consists of the nucleotide sequence.

In some embodiments, the nucleic acid comprises a nucleotide sequence encoding an RNA sequence comprising at least 90% identity to the sequence set forth in any one of SEQ ID NOs: 1-184; the nucleotide sequence encoding the RNA sequence set forth in any one of SEQ ID NOs: 1-184; a nucleotide sequence comprising an RNA sequence comprising at least 90% identity to the sequence set forth in any one of SEQ ID NOs: 1-184; or the nucleotide sequence comprising the RNA sequence set forth in any one of SEQ ID NOs: 1-184. In some aspects, the disclosure comprises a nucleic acid comprising a nucleotide sequence comprising at least 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in set forth in any one of SEQ ID NOs: 1-184. In some aspects, the disclosure comprises a nucleic acid comprising a nucleotide sequence encoding a gRNA comprising at least 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, or 70% identity to the nucleotide sequence set forth in set forth in any one of SEQ ID NOs: 1-184. Exemplary nucleotide sequences of the disclosure include, but are not limited to, those identified in Table 1 below.

Table 1 provides Staphylococcus aureus and Campylobacter jejuni gRNA nucleotide sequences designed to target human and mouse DMD exons and the flanking intronic sequences of the DMD gene. Asterisks after the gRNA ID in column 1 indicate the gRNA targets both mouse and human sequences. The third column in Table 1 provides the gRNA sequences (i.e., SEQ ID NOs: 1-92 comprising both bolded and underlined font) comprising both the unique spacer sequences (i.e., SEQ ID NOs: 93-184, set out in bolded font in columns 3 and 5) and the common repeat:antirepeat gRNA sequences (underlined font in column 3). Table 1 also provides the target nucleotide sequences of the DMD gene.

TABLE 1
Staphylococcus aureus and Campylobacter jejuni gRNA sequences and DMD target sequences.

| | | gRNA sequence (5′-3′) | | | | | |
|---|---|---|---|---|---|---|---|
| | | (comprises UNIQUE SPACER | gRNA | Unique | Spacer | | |
| | | (bold font) + common | SEQ | spacer | SEQ | genomic DNA | Target |
| | Cas | repeat:antirepeat gRNA | ID | sequence | ID | target sequence | SEQ |
| gRNA ID | protein | (underlined font) | NO: | (5′-3′) | NO: | (coding strand) | ID NO: |
| hDSA-001 | S. aureus | GAUCAUACAGUAUUUGAACGA | 1 | GAUCAUACAGUA | 93 | ATCATACAGTAT | 185 |
| | Cas9 | CUGUUUUAGUACUCUGGAAAC | | UUUGAACGACU | | TTGAACGACTAT | |
| | | AGAAUCUACUAAAACAAGGCAA | | | | GGGT | |
| | | AAUGCCGUGUUUAUCUCGUCA | | | | | |
| | | ACUUGUUGGCGAGAUUUUU | | | | | |
| hDSA-002 | S. aureus | GUUCAGAUAUUACAAAUCUGA | 2 | GUUCAGAUAUUA | 94 | TTCAGATATTAC | 186 |
| | Cas9 | GGGUUUUAGUACUCUGGAAAC | | CAAAUCUGAGG | | AAATCTGAGGCG | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| hDSA- | S. aureus | GAUCUUGCUCUGUCGCCCACG | 3 | GAUCUUGCUCU | 95 | GATCTTGCTCTG | 187 |
| 003* | Cas9 | CGUUUUAGUACUCUGGAAACA | | GUCGCCCACGC | | TCGCCCACGCT | |
| | | GAAUCUACUAAAACAAGGCAAA | | | | GGAGT | |
| | | AUGCCGUGUUUAUCUCGUCAA | | | | | |
| | | CUUGUUGGCGAGAUUUUU | | | | | |
| hDSA- | S. aureus | GGCUCACUGCAACCUCCACCU | 4 | GGCUCACUGCA | 96 | GGCTCACTGCAA | 188 |
| 004* | Cas9 | CGUUUUAGUACUCUGGAAACA | | ACCUCCACCUC | | CCTCCACCTCCT | |
| | | GAAUCUACUAAAACAAGGCAAA | | | | GGAT | |
| | | AUGCCGUGUUUAUCUCGUCAA | | | | | |
| | | CUUGUUGGCGAGAUUUUU | | | | | |
| hDSA- | S. aureus | GAGGCUGAGGCAGGAGAAUCA | 5 | GAGGCUGAGGC | 97 | ATTCAAATGATT | 189 |
| 005* | Cas9 | UGUUUUAGUACUCUGGAAACA | | AGGAGAAUCAU | | CTCCTGCCTCAG | |
| | | GAAUCUACUAAAACAAGGCAAA | | | | | |
| | | AUGCCGUGUUUAUCUCGUCAA | | | | CCTC | |
| | | CUUGUUGGCGAGAUUUUU | | | | | |
| hDSA- | S. aureus | GCUACUUGGGAGGCUGAGGCA | 6 | GCUACUUGGGA | 98 | ATTCTCCTGCCT | 190 |
| 006* | Cas9 | GGUUUUAGUACUCUGGAAACA | | GGCUGAGGCAG | | CAGCCTCCCAAG | |
| | | GAAUCUACUAAAACAAGGCAAA | | | | TAGC | |
| | | AUGCCGUGUUUAUCUCGUCAA | | | | | |
| | | CUUGUUGGCGAGAUUUUU | | | | | |
| hDSA- | S. aureus | GCUGCCUCAGCCUCCCAAGUA | 7 | GCUGCCUCAGC | 99 | CTGCCTCAGCCT | 191 |
| 007* | Cas9 | GCGUUUUAGUACUCUGGAAAC | | CUCCCAAGUAGC | | CCCAAGTAGCTG | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| hDSA- | S. aureus | GAUUUUUGUAUUUUCAGUAGA | 8 | GAUUUUUGUAUU | 100 | ATTTTTGTATTT | 192 |
| 008* | Cas9 | GAGUUUUAGUACUCUGGAAAC | | UUCAGUAGAGA | | TCAGTAGAGATG | |
| | | AGAAUCUACUAAAACAAGGCAA | | | | GGGT | |
| | | AAUGCCGUGUUUAUCUCGUCA | | | | | |
| | | ACUUGUUGGCGAGAUUUUU | | | | | |
| hDSA- | S. aureus | GACAGUUUGGGAGGCCAGUGA | 9 | GACAGUUUGGG | 101 | ATCCGTCCTCAC | 193 |
| 009* | Cas9 | GGGUUUUAGUACUCUGGAAAC | | AGGCCAGUGAG | | TGGCCTOCCAAA | |
| | | AGAAUCUACUAAAACAAGGCAA | | G | | CTGT | |
| | | AAUGCCGUGUUUAUCUCGUCA | | | | | |
| | | ACUUGUUGGCGAGAUUUUU | | | | | |
| hDSA- | S. aureus | GCCUCACUGGCCUCCCAAACU | 10 | GCCUCACUGGC | 102 | CCTCACTGGCCT | 194 |
| 010* | Cas9 | GUGUUUUAGUACUCUGGAAAC | | CUCCCAAACUGU | | CCCAAACTGTTG | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| hDSA- | S. aureus | GAUUUUUCAGAAGUAAUUUUA | 11 | GAUUUUUCAGAA | 103 | ATTTTTCAGAAG | 195 |
| 011* | Cas9 | AUGUUUUAGUACUCUGGAAAC | | GUAAUUUUAAU | | TAATTTTAATTTG | |
| | | AGAAUCUACUAAAACAAGGCAA | | | | GAT | |
| | | AAUGCCGUGUUUAUCUCGUCA | | | | | |
| | | ACUUGUUGGCGAGAUUUUU | | | | | |
| hDSA-012 | S. aureus | GUCAUUGAUAAAUGGAAUUAA | 12 | GUCAUUGAUAAA | 104 | ACTCATGTTTAA | 196 |
| | Cas9 | ACGUUUUAGUACUCUGGAAAC | | UGGAAUUAAAC | | TTCCATTTATCAA | |
| | | AGAAUCUACUAAAACAAGGCAA | | | | TGA | |
| | | AAUGCCGUGUUUAUCUCGUCA | | | | | |
| | | ACUUGUUGGCGAGAUUUUU | | | | | |
| hDSA-013 | S. aureus | GUCAUGUUUAAUUCCAUUUAU | 13 | GUCAUGUUUAAU | 105 | TCATGTTTAATTC | 197 |
| | Cas9 | CAGUUUUAGUACUCUGGAAAC | | UCCAUUUAUCA | | CATTTATCAATG | |
| | | AGAAUCUACUAAAACAAGGCAA | | | | AAT | |
| | | AAUGCCGUGUUUAUCUCGUCA | | | | | |
| | | ACUUGUUGGCGAGAUUUUU | | | | | |
| hDSA-014 | S. aureus | GUAGUAUUACAUUCAUUGAUA | 14 | GUAGUAUUACAU | 106 | ATTCCATTTATCA | 198 |
| | Cas9 | AAGUUUUAGUACUCUGGAAAC | | UCAUUGAUAAA | | ATGAATGTAATA | |
| | | AGAAUCUACUAAAACAAGGCAA | | | | CTA | |
| | | AAUGCCGUGUUUAUCUCGUCA | | | | | |
| | | ACUUGUUGGCGAGAUUUUU | | | | | |
| hDSA-015 | S. aureus | GAAAGUUACUUUGGUUGUAAA | 15 | GAAAGUUACUUU | 107 | AAAGTTACTTTG | 199 |
| | Cas9 | AUGUUUUAGUACUCUGGAAAC | | GGUUGUAAAAU | | GTTGTAAAATAT | |
| | | AGAAUCUACUAAAACAAGGCAA | | | | GAAT | |
| | | AAUGCCGUGUUUAUCUCGUCA | | | | | |
| | | ACUUGUUGGCGAGAUUUUU | | | | | |
| hDSA- | S. aureus | GUUCAAAAGAAAACAUUCACA | 16 | GUUCAAAAGAAA | 108 | TTCAAAAGAAAA | 200 |
| 016* | Cas9 | AAGUUUUAGUACUCUGGAAAC | | ACAUUCACAAA | | CATTCACAAAAT | |
| | | AGAAUCUACUAAAACAAGGCAA | | | | GGGT | |
| | | AAUGCCGUGUUUAUCUCGUCA | | | | | |
| | | ACUUGUUGGCGAGAUUUUU | | | | | |
| hDSA-017 | S. aureus | GAAAUUGUGCAUUUACCCAUU | 17 | GAAAUUGUGCA | 109 | ATTCACAAAATG | 201 |
| | Cas9 | UUGUUUUAGUACUCUGGAAAC | | UUUACCCAUUUU | | GGTAAATGCACA | |
| | | AGAAUCUACUAAAACAAGGCAA | | | | ATTT | |
| | | AAUGCCGUGUUUAUCUCGUCA | | | | | |
| | | ACUUGUUGGCGAGAUUUUU | | | | | |
| hDSA-018 | S. aureus | GUAAAUGCACAAUUUUCUAAG | 18 | GUAAAUGCACAA | 110 | TAAATGCACAAT | 202 |
| | Cas9 | GUGUUUUAGUACUCUGGAAAC | | UUUUCUAAGGU | | TTTCTAAGGTAA | |
| | | AGAAUCUACUAAAACAAGGCAA | | | | GAAT | |
| | | AAUGCCGUGUUUAUCUCGUCA | | | | | |
| | | ACUUGUUGGCGAGAUUUUU | | | | | |
| hDSA-019 | S. aureus | GAUGACACUAUGAGAGAAAUA | 19 | GAUGACACUAU | 111 | ATCCGTTTTATTT | 203 |
| | Cas9 | AAGUUUUAGUACUCUGGAAAC | | GAGAGAAAUAA | | CTCTCATAGTGT | |
| | | AGAAUCUACUAAAACAAGGCAA | | A | | CAT | |
| | | AAUGCCGUGUUUAUCUCGUCA | | | | | |
| | | ACUUGUUGGCGAGAUUUUU | | | | | |
| hDSA-020 | S. aureus | GAUAUUAUGUUCACUCUUAUU | 20 | GAUAUUAUGUUC | 112 | ATATTATGTTCA | 204 |
| | Cas9 | UAGUUUUAGUACUCUGGAAAC | | ACUCUUAUUUA | | CTCTTATTTAAG | |
| | | AGAAUCUACUAAAACAAGGCAA | | | | GAGT | |
| | | AAUGCCGUGUUUAUCUCGUCA | | | | | |
| | | ACUUGUUGGCGAGAUUUUU | | | | | |
| hDSA-021 | S. aureus | GUUCUGCUGCUUACUCCUUAA | 21 | GUUCUGCUGCU | 113 | ACTCTTATTTAA | 205 |
| | Cas9 | AUGUUUUAGUACUCUGGAAAC | | UACUCCUUAAAU | | GGAGTAAGCAG | |
| | | AGAAUCUACUAAAACAAGGCAA | | | | CAGAA | |
| | | AAUGCCGUGUUUAUCUCGUCA | | | | | |
| | | ACUUGUUGGCGAGAUUUUU | | | | | |
| hDSA-022 | S. aureus | GAUCUUUGAUUCUUCUUGACA | 22 | GAUCUUUGAUUC | 114 | ATCTTTGATTCTT | 206 |
| | Cas9 | AUGUUUUAGUACUCUGGAAAC | | UUCUUGACAAU | | CTTGACAATGTG | |
| | | AGAAUCUACUAAAACAAGGCAA | | | | AGT | |
| | | AAUGCCGUGUUUAUCUCGUCA | | | | | |
| | | ACUUGUUGGCGAGAUUUUU | | | | | |
| hDSA- | S. aureus | GUAAAGAAACUCACAUUGUCA | 23 | GUAAAGAAACUC | 115 | ATTCTTCTTGAC | 207 |
| 023* | Cas9 | AGGUUUUAGUACUCUGGAAAC | | ACAUUGUCAAG | | AATGTGAGTTTC | |
| | | AGAAUCUACUAAAACAAGGCAA | | | | TTTA | |
| | | AAUGCCGUGUUUAUCUCGUCA | | | | | |
| | | ACUUGUUGGCGAGAUUUUU | | | | | |
| hDSA- | S. aureus | GCAAUGUGAGUUUCUUUAAUG | 24 | GCAAUGUGAGU | 116 | CAATGTGAGTTT | 208 |
| 024* | Cas9 | ACGUUUUAGUACUCUGGAAAC | | UUCUUUAAUGAC | | CTTTAATGACAT | |
| | | AGAAUCUACUAAAACAAGGCAA | | | | | |
| | | AAUGCCGUGUUUAUCUCGUCA | | | | GAGT | |
| | | ACUUGUUGGCGAGAUUUUU | | | | | |
| hDSA-025 | S. aureus | GUUUCUUUAAUGACAUGAGUC | 25 | GUUUCUUUAAUG | 117 | TTTCTTTAATGAC | 209 |
| | Cas9 | CUGUUUUAGUACUCUGGAAAC | | ACAUGAGUCCU | | ATGAGTCCTCAG | |
| | | AGAAUCUACUAAAACAAGGCAA | | | | AGT | |
| | | AAUGCCGUGUUUAUCUCGUCA | | | | | |
| | | ACUUGUUGGCGAGAUUUUU | | | | | |
| hDSA- | S. aureus | GCUGCAAUCUUCCAUCAAGUA | 26 | GCUGCAAUCUUC | 118 | CTGCAATCTTCC | 210 |
| 026* | Cas9 | GCGUUUUAGUACUCUGGAAAC | | CAUCAAGUAGC | | ATCAAGTAGCTA | |
| | | AGAAUCUACUAAAACAAGGCAA | | | | GAAT | |
| | | AAUGCCGUGUUUAUCUCGUCA | | | | | |
| | | ACUUGUUGGCGAGAUUUUU | | | | | |
| hDSA-027 | S. aureus | GCACCCAGCAGAAGAAGAUAU | 27 | GCACCCAGCAG | 119 | CACCCAGCAGAA | 211 |
| | Cas9 | GAGUUUUAGUACUCUGGAAAC | | AAGAAGAUAUG | | GAAGATATGAGG | |
| | | AGAAUCUACUAAAACAAGGCAA | | A | | GAAT | |
| | | AAUGCCGUGUUUAUCUCGUCA | | | | | |
| | | ACUUGUUGGCGAGAUUUUU | | | | | |
| hDSA-028 | S. aureus | GAAUUCCCUCAUAUCUUCUUC | 28 | GAAUUCCCUCAU | 120 | ACCCAGCAGAA | 212 |
| | Cas9 | UGGUUUUAGUACUCUGGAAAC | | AUCUUCUUCUG | | GAAGATATGAGG | |
| | | AGAAUCUACUAAAACAAGGCAA | | | | GAATT | |
| | | AAUGCCGUGUUUAUCUCGUCA | | | | | |
| | | ACUUGUUGGCGAGAUUUUU | | | | | |
| hDSA-029 | S. aureus | GCAAGAAAAGCAAUAUAAUUG | 29 | GCAAGAAAAGC | 121 | CAAGAAAAGCAA | 213 |
| | Cas9 | CUGUUUUAGUACUCUGGAAAC | | AAUAUAAUUGCU | | TATAATTGCTTT | |
| | | AGAAUCUACUAAAACAAGGCAA | | | | GGGT | |
| | | AAUGCCGUGUUUAUCUCGUCA | | | | | |
| | | ACUUGUUGGCGAGAUUUUU | | | | | |
| hDSA-030 | S. aureus | GAAUAUGCUCUAAACUAUAGU | 30 | GAAUAUGCUCUA | 122 | ATTCAGCCACTA | 214 |
| | Cas9 | GGGUUUUAGUACUCUGGAAAC | | AACUAUAGUGG | | TAGTTTAGAGCA | |
| | | AGAAUCUACUAAAACAAGGCAA | | | | TATT | |
| | | AAUGCCGUGUUUAUCUCGUCA | | | | | |
| | | ACUUGUUGGCGAGAUUUUU | | | | | |
| hDSA- | S. aureus | GAAAUUAAGUAAAUAUUAAGA | 31 | GAAAUUAAGUAA | 123 | ATCCATACTCTT | 215 |
| 031* | Cas9 | GUGUUUUAGUACUCUGGAAAC | | AUAUUAAGAGU | | AATATTTACTTAA | |
| | | AGAAUCUACUAAAACAAGGCAA | | | | TTT | |
| | | AAUGCCGUGUUUAUCUCGUCA | | | | | |
| | | ACUUGUUGGCGAGAUUUUU | | | | | |
| hDSA- | S. aureus | GAUUUGCAAAUUAAGUAAAUA | 32 | GAUUUGCAAAUU | 124 | ACTCTTAATATTT | 216 |
| 032* | Cas9 | UUGUUUUAGUACUCUGGAAAC | | AAGUAAAUAUU | | ACTTAATTTGCA | |
| | | AGAAUCUACUAAAACAAGGCAA | | | | AAT | |
| | | AAUGCCGUGUUUAUCUCGUCA | | | | | |
| | | ACUUGUUGGCGAGAUUUUU | | | | | |
| hDSA-033 | S. aureus | GAGUCACGCUAUUGAUUAAAA | 33 | GAGUCACGCUA | 125 | ACTCAATTTTTAA | 217 |
| | Cas9 | AGUUUUAGUACUCUGGAAACA | | UUGAUUAAAAA | | TCAATAGCGTGA | |
| | | GAAUCUACUAAAACAAGGCAAA | | | | CTC | |
| | | AUGCCGUGUUUAUCUCGUCAA | | | | | |
| | | CUUGUUGGCGAGAUUUUU | | | | | |
| mDSA- | S. aureus | GUGGUAUUGAACAUUCAAUUA | 34 | GUGGUAUUGAA | 126 | ATTCTTTTTAATT | 218 |
| 001 | Cas9 | AAGUUUUAGUACUCUGGAAAC | | CAUUCAAUUAAA | | GAATGTTCAATA | |
| | | AGAAUCUACUAAAACAAGGCAA | | | | CCA | |
| | | AAUGCCGUGUUUAUCUCGUCA | | | | | |
| | | ACUUGUUGGCGAGAUUUUU | | | | | |
| mDSA- | S. aureus | GUUAAUUUAGAAAUUCUUUUU | 35 | GUUAAUUUAGAA | 127 | TTAATTTAGAAAT | 219 |
| 002* | Cas9 | AAGUUUUAGUACUCUGGAAAC | | AUUCUUUUUAA | | TCTTTTTAATTGA | |
| | | AGAAUCUACUAAAACAAGGCAA | | | | AT | |
| | | AAUGCCGUGUUUAUCUCGUCA | | | | | |
| | | ACUUGUUGGCGAGAUUUUU | | | | | |
| mDSA- | S. aureus | GAAUAAAAAAUAAAAUUUAUA | 36 | GAAUAAAAAAUA | 128 | ATCCTAGTTATA | 220 |
| 003* | Cas9 | ACGUUUUAGUACUCUGGAAAC | | AAAUUUAUAAC | | AATTTTATTTTTT | |
| | | AGAAUCUACUAAAACAAGGCAA | | | | ATT | |
| | | AAUGCCGUGUUUAUCUCGUCA | | | | | |
| | | ACUUGUUGGCGAGAUUUUU | | | | | |
| mDSA- | S. aureus | GAAAUUGUGCAUUUAUCCAUU | 37 | GAAAUUGUGCA | 129 | ATTCACAAAATG | 221 |
| 004 | Cas9 | UUGUUUUAGUACUCUGGAAAC | | UUUAUCCAUUUU | | GATAAATGCACA | |
| | | AGAAUCUACUAAAACAAGGCAA | | | | ATTT | |
| | | AAUGCCGUGUUUAUCUCGUCA | | | | | |
| | | ACUUGUUGGCGAGAUUUUU | | | | | |
| mDSA- | S. aureus | GUUCAAAAGAAAACAUUCACA | 38 | GUUCAAAAGAAA | 130 | TTCAAAAGAAAA | 222 |
| 005* | Cas9 | AAGUUUUAGUACUCUGGAAAC | | ACAUUCACAAA | | CATTCACAAAAT | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| mDSA- | S. aureus | GUCCACAUUUCAAAUUAUAGC | 39 | GUCCACAUUUCA | 131 | ATTCTAGAGCTA | 223 |
| 007 | Cas9 | UCGUUUUAGUACUCUGGAAAC | | AAUUAUAGCUC | | TAATTTGAAATG | |
| | | AGAAUCUACUAAAACAAGGCAA | | | | TGGA | |
| | | AAUGCCGUGUUUAUCUCGUCA | | | | | |
| | | ACUUGUUGGCGAGAUUUUU | | | | | |
| mDSA- | S. aureus | GUUCUAGAGCUAUAAUUUGAA | 40 | GUUCUAGAGCU | 132 | TTCTAGAGCTAT | 224 |
| 00 | Cas9 | AUGUUUUAGUACUCUGGAAAC | | AUAAUUUGAAAU | | AATTTGAAATGT | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| mDSA- | S. aureus | GUUUCUUAUGUGUCAUUUUAA | 41 | GUUUCUUAUGU | 133 | TTTCTTATGTGT | 225 |
| 009 | Cas9 | UUGUUUUAGUACUCUGGAAAC | | GUCAUUUUAAUU | | CATTTTAATTTTG | |
| | | AGAAUCUACUAAAACAAGGCAA | | | | GAT | |
| | | AAUGCCGUGUUUAUCUCGUCA | | | | | |
| | | ACUUGUUGGCGAGAUUUUU | | | | | |
| mDSA- | S. aureus | GUUUAAAACAUAUGUAAAGAG | 42 | GUUUAAAACAUA | 134 | TTTAAAACATAT | 226 |
| 010 | Cas9 | GUGUUUUAGUACUCUGGAAAC | | UGUAAAGAGGU | | GTAAAGAGGTG | |
| | | AGAAUCUACUAAAACAAGGCAA | | | | GGAAT | |
| | | AAUGCCGUGUUUAUCUCGUCA | | | | | |
| | | ACUUGUUGGCGAGAUUUUU | | | | | |
| hDCJ-001 | C. jejuni | GUUAUCUUUGCUUCCCAGAAC | 43 | GUUAUCUUUGCU | 135 | GTGTGCTAAGTT | 227 |
| | Cas9 | UGUUUUAGUCCCUGAAAAGGG | | UCCCAGAACU | | CTGGGAAGCAAA | |
| | | ACUAAAAUAAAGAGUUUGCGG | | | | | |
| | | GACUCUGCGGGGUUACAAUCC | | | | GATAAC | |
| | | CCUAAAACCGCUUUUUUU | | | | | |
| hDCJ-002 | C. jejuni | GUAGUUAUCUUUGCUUCCCAG | 44 | GUAGUUAUCUUU | 136 | GTGCTAAGTTCT | 228 |
| | Cas9 | AAGUUUUAGUCCCUGAAAAGG | | GCUUCCCAGAA | | GGGAAGCAAAG | |
| | | GACUAAAAUAAAGAGUUUGCG | | | | ATAACTA | |
| | | GGACUCUGCGGGGUUACAAUC | | | | | |
| | | CCCUAAAACCGCUUUUUUU | | | | | |
| hDCJ-003 | C. jejuni | GAUUAUUCCUUCCUUUGCAGC | 45 | GAUUAUUCCUUC | 137 | GTATGTAGATGC | 229 |
| | Cas9 | AUGUUUUAGUCCCUGAAAAGG | | CUUUGCAGCAU | | TGCAAAGGAAG | |
| | | GACUAAAAUAAAGAGUUUGCG | | | | GAATAAT | |
| | | GGACUCUGCGGGGUUACAAUC | | | | | |
| | | CCCUAAAACCGCUUUUUUU | | | | | |
| hDCJ-004 | C. jejuni | GAUGACAGCUGAGAGUUUCUU | 46 | GAUGACAGCUG | 138 | ATGACAGCTGAG | 230 |
| | Cas9 | UUGUUUUAGUCCCUGAAAAGG | | AGAGUUUCUUUU | | AGTTTCTTTTAAT | |
| | | GACUAAAAUAAAGAGUUUGCG | | | | TATAC | |
| | | GGACUCUGCGGGGUUACAAUC | | | | | |
| | | CCCUAAAACCGCUUUUUUU | | | | | |
| hDCJ- | C. jejuni | GAGUAUCUAUUCAUAACUGGC | 47 | GAGUAUCUAUUC | 139 | AGTATCTATTCA | 231 |
| 005* | Cas9 | GAGUUUUAGUCCCUGAAAAGG | | AUAACUGGCGA | | TAACTGGCGAAT | |
| | | GACUAAAAUAAAGAGUUUGCG | | | | AAATAC | |
| | | GGACUCUGCGGGGUUACAAUC | | | | | |
| | | CCCUAAAACCGCUUUUUUU | | | | | |
| hDCJ- | C. jejuni | GAGUAUUUAUUCGCCAGUUAU | 48 | GAGUAUUUAUUC | 140 | GTATCTATTCAT | 232 |
| 006* | Cas9 | GAGUUUUAGUCCCUGAAAAGG | | GCCAGUUAUGA | | AACTGGCGAATA | |
| | | GACUAAAAUAAAGAGUUUGCG | | | | AATACT | |
| | | GGACUCUGCGGGGUUACAAUC | | | | | |
| | | CCCUAAAACCGCUUUUUUU | | | | | |
| hDCJ- | C. jejuni | GUCAGAGGAGACUAUUUUUUU | 49 | GUCAGAGGAGA | 141 | TCAGAGGAGACT | 233 |
| 007* | Cas9 | AUGUUUUAGUCCCUGAAAAGG | | CUAUUUUUUUAU | | ATTTTTTTATAAT | |
| | | GACUAAAAUAAAGAGUUUGCG | | | | CATAC | |
| | | GGACUCUGCGGGGUUACAAUC | | | | | |
| | | CCCUAAAACCGCUUUUUUU | | | | | |
| hDCJ-008 | C. jejuni | GAUAUGACCAAACCCAUAGUC | 50 | GAUAUGACCAAA | 142 | GTATTTGAACGA | 234 |
| | Cas9 | GUGUUUUAGUCCCUGAAAAGG | | CCCAUAGUCGU | | CTATGGGTTTGG | |
| | | GACUAAAAUAAAGAGUUUGCG | | | | TCATAT | |
| | | GGACUCUGCGGGGUUACAAUC | | | | | |
| | | CCCUAAAACCGCUUUUUUU | | | | | |
| hDCJ- | C. jejuni | GUUGCAGUGAGCCGAGAUCAU | 51 | GUUGCAGUGAG | 143 | GTGCAGTGCCAT | 235 |
| 009* | Cas9 | GGGUUUUAGUCCCUGAAAAGG | | CCGAGAUCAUG | | GATCTCGGCTCA | |
| | | GACUAAAAUAAAGAGUUUGCG | | G | | CTGCAA | |
| | | GGACUCUGCGGGGUUACAAUC | | | | | |
| | | CCCUAAAACCGCUUUUUUU | | | | | |
| hDCJ- | C. jejuni | GGAGGUUGCAGUGAGCCGAG | 52 | GGAGGUUGCAG | 144 | GTGCCATGATCT | 236 |
| 010* | Cas9 | AUGUUUUAGUCCCUGAAAAGG | | UGAGCCGAGAU | | CGGCTCACTGCA | |
| | | GACUAAAAUAAAGAGUUUGCG | | | | ACCTCC | |
| | | GGACUCUGCGGGGUUACAAUC | | | | | |
| | | CCCUAAAACCGCUUUUUUU | | | | | |
| hDCJ- | C. jejuni | GCAAAAAUUAGCCAGGCAUGG | 53 | GCAAAAAUUAGC | 145 | GTGCATGCCACC | 237 |
| 011* | Cas9 | UGGUUUUAGUCCCUGAAAAGG | | CAGGCAUGGUG | | ATGCCTGGCTAA | |
| | | GACUAAAAUAAAGAGUUUGCG | | | | TTTTTG | |
| | | GGACUCUGCGGGGUUACAAUC | | | | | |
| | | CCCUAAAACCGCUUUUUUU | | | | | |
| hDCJ- | C. jejuni | GAUGGCGAAACCCCAUCUCUA | 54 | GAUGGCGAAAC | 146 | GTATTTTCAGTA | 238 |
| 012* | Cas9 | CUGUUUUAGUCCCUGAAAAGG | | CCCAUCUCUACU | | GAGATGGGGTTT | |
| | | GACUAAAAUAAAGAGUUUGCG | | | | CGCCAT | |
| | | GGACUCUGCGGGGUUACAAUC | | | | | |
| | | CCCUAAAACCGCUUUUUUU | | | | | |
| hDCJ-013 | C. jejuni | GUUUAAUUCCAUUUAUCAAUG | 55 | GUUUAAUUCCAU | 147 | TTTAATTCCATTT | 239 |
| | Cas9 | AAGUUUUAGUCCCUGAAAAGG | | UUAUCAAUGAA | | ATCAATGAATGT | |
| | | GACUAAAAUAAAGAGUUUGCG | | | | AATAC | |
| | | GGACUCUGCGGGGUUACAAUC | | | | | |
| | | CCCUAAAACCGCUUUUUUU | | | | | |
| hDCJ-014 | C. jejuni | GAUGAAUGUAAUACUAAAUGU | 56 | GAUGAAUGUAA | 148 | ATGAATGTAATA | 240 |
| | Cas9 | AAGUUUUAGUCCCUGAAAAGG | | UACUAAAUGUAA | | CTAAATGTAAAA | |
| | | GACUAAAAUAAAGAGUUUGCG | | | | AAACAC | |
| | | GGACUCUGCGGGGUUACAAUC | | | | | |
| | | CCCUAAAACCGCUUUUUUU | | | | | |
| hDCJ-015 | C. jejuni | GUAAUACUAAAUGUAAAAAAA | 57 | GUAAUACUAAAU | 149 | GTAATACTAAAT | 241 |
| | Cas9 | CGUUUUAGUCCCUGAAAAGGG | | GUAAAAAAAC | | GTAAAAAAACAC | |
| | | ACUAAAAUAAAGAGUUUGCGG | | | | TAACAC | |
| | | GACUCUGCGGGGUUACAAUCC | | | | | |
| | | CCUAAAACCGCUUUUUUU | | | | | |
| hDCJ- | C. jejuni | GAGAAAACAUUCACAAAAUGG | 58 | GAGAAAACAUUC | 150 | AGAAAACATTCA | 242 |
| 016* | Cas9 | GUGUUUUAGUCCCUGAAAAGG | | ACAAAAUGGGU | | CAAAATGGGTAA | |
| | | GACUAAAAUAAAGAGUUUGCG | | | | ATGCAC | |
| | | GGACUCUGCGGGGUUACAAUC | | | | | |
| | | CCCUAAAACCGCUUUUUUU | | | | | |
| hDCJ-017 | C. jejuni | GUUGUGAAAUUUUCAAAAUGG | 59 | GUUGUGAAAUU | 151 | GTTGTGAAATTT | 243 |
| | Cas9 | AGUUUUAGUCCCUGAAAAGGG | | UUCAAAAUGGA | | TCAAAATGGACT | |
| | | ACUAAAAUAAAGAGUUUGCGG | | | | ATGTAC | |
| | | GACUCUGCGGGGUUACAAUCC | | | | | |
| | | CCUAAAACCGCUUUUUUU | | | | | |
| hDCJ-018 | C. jejuni | GAUAAAACGGAUUUUUAAGAU | 60 | GAUAAAACGGA | 152 | GTACCTGTGTAT | 244 |
| | Cas9 | ACGUUUUAGUCCCUGAAAAGG | | UUUUUAAGAUAC | | CTTAAAAATCCG | |
| | | GACUAAAAUAAAGAGUUUGCG | | | | TTTTAT | |
| | | GGACUCUGCGGGGUUACAAUC | | | | | |
| | | CCCUAAAACCGCUUUUUUU | | | | | |
| hDCJ-019 | C. jejuni | GAGAGAAAUAAAACGGAUUUU | 61 | GAGAGAAAUAA | 153 | GTGTATOTTAAA | 245 |
| | Cas9 | UAGUUUUAGUCCCUGAAAAGG | | AACGGAUUUUUA | | AATCCGTTTTATT | |
| | | GACUAAAAUAAAGAGUUUGCG | | | | TCTCT | |
| | | GGACUCUGCGGGGUUACAAUC | | | | | |
| | | CCCUAAAACCGCUUUUUUU | | | | | |
| hDCJ-020 | C. jejuni | GUGAGAGAAAUAAAACGGAUU | 62 | GUGAGAGAAAU | 154 | GTATCTTAAAAA | 246 |
| | Cas9 | UUGUUUUAGUCCCUGAAAAGG | | AAAACGGAUUUU | | TCCGTTTTATTTC | |
| | | GACUAAAAUAAAGAGUUUGCG | | | | | |
| | | GGACUCUGCGGGGUUACAAUC | | | | TCTCA | |
| | | CCCUAAAACCGCUUUUUUU | | | | | |
| hDCJ-021 | C. jejuni | GCAGAUUUGCACAGCUAAAAU | 63 | GCAGAUUUGCA | 155 | GTGTCATTTTATT | 247 |
| | Cas9 | AAGUUUUAGUCCCUGAAAAGG | | CAGCUAAAAUAA | | TTAGCTGTGCAA | |
| | | GACUAAAAUAAAGAGUUUGCG | | | | ATCTG | |
| | | GGACUCUGCGGGGUUACAAUC | | | | | |
| | | CCCUAAAACCGCUUUUUUU | | | | | |
| hDCJ-022 | C. jejuni | GAGAGUGAACAUAAUAUUUCC | 64 | GAGAGUGAACA | 156 | GTGCAAATCTGG | 248 |
| | Cas9 | AGGUUUUAGUCCCUGAAAAGG | | UAAUAUUUCCAG | | AAATATTATGTTC | |
| | | GACUAAAAUAAAGAGUUUGCG | | | | ACTCT | |
| | | GGACUCUGCGGGGUUACAAUC | | | | | |
| | | CCCUAAAACCGCUUUUUUU | | | | | |
| hDCJ-023 | C. jejuni | GAGUAAGCAGCAGAAGAUAUG | 65 | GAGUAAGCAGC | 157 | AGTAAGCAGCAG | 249 |
| | Cas9 | GCGUUUUAGUCCCUGAAAAGG | | AGAAGAUAUGG | | AAGATATGGCAA | |
| | | GACUAAAAUAAAGAGUUUGCG | | C | | AGATAC | |
| | | GGACUCUGCGGGGUUACAAUC | | | | | |
| | | CCCUAAAACCGCUUUUUUU | | | | | |
| hDCJ-024 | C. jejuni | GUAAGCAGCAGAAGAUAUGGC | 66 | GUAAGCAGCAG | 158 | TAAGCAGCAGAA | 250 |
| | Cas9 | AAGUUUUAGUCCCUGAAAAGG | | AAGAUAUGGCA | | GATATGGCAAAG | |
| | | GACUAAAAUAAAGAGUUUGCG | | A | | ATACAC | |
| | | GGACUCUGCGGGGUUACAAUC | | | | | |
| | | CCCUAAAACCGCUUUUUUU | | | | | |
| hDCJ-025 | C. jejuni | GUCACAUUGUCAAGAAGAAUC | 67 | GUCACAUUGUCA | 159 | GTATATCTTTGA | 251 |
| | Cas9 | AAGUUUUAGUCCCUGAAAAGG | | AGAAGAAUCAA | | TTCTTCTTGACA | |
| | | GACUAAAAUAAAGAGUUUGCG | | | | ATGTGA | |
| | | GGACUCUGCGGGGUUACAAUC | | | | | |
| | | CCCUAAAACCGCUUUUUUU | | | | | |
| hDCJ-026 | C. jejuni | GUAUGCUCUAAACUAUAGUGG | 68 | GUAUGCUCUAAA | 160 | GTGTATTCAGCC | 252 |
| | Cas9 | CUGUUUUAGUCCCUGAAAAGG | | CUAUAGUGGCU | | ACTATAGTTTAG | |
| | | GACUAAAAUAAAGAGUUUGCG | | | | AGCATA | |
| | | GGACUCUGCGGGGUUACAAUC | | | | | |
| | | CCCUAAAACCGCUUUUUUU | | | | | |
| hDCJ-027 | C. jejuni | GAAUAUGCUCUAAACUAUAGU | 69 | GAAUAUGCUCUA | 161 | GTATTCAGCCAC | 253 |
| | Cas9 | GGGUUUUAGUCCCUGAAAAGG | | AACUAUAGUGG | | TATAGTTTAGAG | |
| | | GACUAAAAUAAAGAGUUUGCG | | | | CATATT | |
| | | GGACUCUGCGGGGUUACAAUC | | | | | |
| | | CCCUAAAACCGCUUUUUUU | | | | | |
| hDCJ-028 | C. jejuni | GUCUUGUUUUUGUGCAGGCUU | 70 | GUCUUGUUUUU | 162 | TCTTGTTTTTGT | 254 |
| | Cas9 | CAGUUUUAGUCCCUGAAAAGG | | GUGCAGGCUUC | | GCAGGCTTCAAT | |
| | | GACUAAAAUAAAGAGUUUGCG | | A | | CCATAC | |
| | | GGACUCUGCGGGGUUACAAUC | | | | | |
| | | CCCUAAAACCGCUUUUUUU | | | | | |
| hDCJ-029 | C. jejuni | GAAUAUUAAGAGUAUGGAUUG | 71 | GAAUAUUAAGA | 163 | GTGCAGGCTTCA | 255 |
| | Cas9 | AAGUUUUAGUCCCUGAAAAGG | | GUAUGGAUUGA | | ATCCATACTCTT | |
| | | GACUAAAAUAAAGAGUUUGCG | | A | | AATATT | |
| | | GGACUCUGCGGGGUUACAAUC | | | | | |
| | | CCCUAAAACCGCUUUUUUU | | | | | |
| hDCJ-030 | C. jejuni | GACUCUUAAUAUUUACUUAAU | 72 | GACUCUUAAUAU | 164 | ACTCTTAATATTT | 256 |
| | Cas9 | UUGUUUUAGUCCCUGAAAAGG | | UUACUUAAUUU | | ACTTAATTTGCA | |
| | | GACUAAAAUAAAGAGUUUGCG | | | | AATAC | |
| | | GGACUCUGCGGGGUUACAAUC | | | | | |
| | | CCCUAAAACCGCUUUUUUU | | | | | |
| hDCJ-031 | C. jejuni | GCGUGACUCUAGAUGAUUAUA | 73 | GCGUGACUCUA | 165 | GCGTGACTCTAG | 257 |
| | Cas9 | GGUUUUAGUCCCUGAAAAGGG | | GAUGAUUAUAG | | ATGATTATAGGT | |
| | | ACUAAAAUAAAGAGUUUGCGG | | | | GGACAC | |
| | | GACUCUGCGGGGUUACAAUCC | | | | | |
| | | CCUAAAACCGCUUUUUUU | | | | | |
| mDCJ-001 | C. jejuni | GCUAAAUUAAUAGAUAUGUUC | 74 | GCUAAAUUAAUA | 166 | GTATGGACCTGA | 258 |
| | Cas9 | AGGUUUUAGUCCCUGAAAAGG | | GAUAUGUUCAG | | ACATATCTATTAA | |
| | | GACUAAAAUAAAGAGUUUGCG | | | | TTTAG | |
| | | GGACUCUGCGGGGUUACAAUC | | | | | |
| | | CCCUAAAACCGCUUUUUUU | | | | | |
| mDCJ-002 | C. jejuni | GAGAAAUUCUUUUUAAUUGAA | 75 | GAGAAAUUCUUU | 167 | AGAAATTCTTTTT | 259 |
| | Cas9 | UGGUUUUAGUCCCUGAAAAGG | | UUAAUUGAAUG | | AATTGAATGTTC | |
| | | GACUAAAAUAAAGAGUUUGCG | | | | AATAC | |
| | | GGACUCUGCGGGGUUACAAUC | | | | | |
| | | CCCUAAAACCGCUUUUUUU | | | | | |
| mDCJ-003 | C. jejuni | GAGAAAACAUUCACAAAAUGG | 76 | GAGAAAACAUUC | 168 | AGAAAACATTCA | 260 |
| | Cas9 | AUGUUUUAGUCCCUGAAAAGG | | ACAAAAUGGAU | | CAAAATGGATAA | |
| | | GACUAAAAUAAAGAGUUUGCG | | | | ATGCAC | |
| | | GGACUCUGCGGGGUUACAAUC | | | | | |
| | | CCCUAAAACCGCUUUUUUU | | | | | |
| mDCJ-004 | C. jejuni | GUAGAAUCAAUUUCAAGAAGU | 77 | GUAGAAUCAAUU | 169 | GTATTGATTGAC | 261 |
| | Cas9 | CAGUUUUAGUCCCUGAAAAGG | | UCAAGAAGUCA | | TTCTTGAAATTG | |
| | | GACUAAAAUAAAGAGUUUGCG | | | | ATTCTA | |
| | | GGACUCUGCGGGGUUACAAUC | | | | | |
| | | CCCUAAAACCGCUUUUUUU | | | | | |
| mDCJ-005 | C. jejuni | GAGACUUGCAGAUCCAAAAUU | 78 | GAGACUUGCAG | 170 | GTGTCATTTTAA | 262 |
| | Cas9 | AAGUUUUAGUCCCUGAAAAGG | | AUCCAAAAUUAA | | TTTTGGATCTGC | |
| | | GACUAAAAUAAAGAGUUUGCG | | | | AAGTCT | |
| | | GGACUCUGCGGGGUUACAAUC | | | | | |
| | | CCCUAAAACCGCUUUUUUU | | | | | |
| JHI3SA001 | S. aureus | GAGAAAGUUAACUUUAAACUU | 79 | GAGAAAGUUAA | 171 | AGAAAGTTAACT | 263 |
| | Cas9 | AGGUUUUAGUACUCUGGAAAC | | CUUUAAACUUAG | | TTAAACTTAGTA | |
| | | AGAAUCUACUAAAACAAGGCAA | | | | GAAT | |
| | | AAUGCCGUGUUUAUCUCGUCA | | | | | |
| | | ACUUGUUGGCGAGAUUUUU | | | | | |
| JHI3SA002 | S. aureus | GAACAUAGUUUUAUGACAUCU | 80 | GAACAUAGUUUU | 172 | AACATAGTTTTAT | 264 |
| | Cas9 | AGGUUUUAGUACUCUGGAAAC | | AUGACAUCUAG | | GACATCTAGTAG | |
| | | AGAAUCUACUAAAACAAGGCAA | | | | AAT | |
| | | AAUGCCGUGUUUAUCUCGUCA | | | | | |
| | | ACUUGUUGGCGAGAUUUUU | | | | | |
| JHI3SA003 | S. aureus | GAUCAUACGAGGUUGCUUUAC | 81 | GAUCAUACGAG | 173 | ATTCCTTAGTAA | 265 |
| | Cas9 | UAGUUUUAGUACUCUGGAAAC | | GUUGCUUUACUA | | AGCAACCTCGTA | |
| | | AGAAUCUACUAAAACAAGGCAA | | | | | |
| | | AAUGCCGUGUUUAUCUCGUCA | | | | TGAT | |
| | | ACUUGUUGGCGAGAUUUUU | | | | | |
| JHI3SA006 | S. aureus | GAGUUAUUUUACAAGGAACCU | 82 | GAGUUAUUUUAC | 174 | AGTTATTTTACAA | 266 |
| | Cas9 | GUGUUUUAGUACUCUGGAAAC | | AAGGAACCUGU | | GGAACCTGTATG | |
| | | AGAAUCUACUAAAACAAGGCAA | | | | AGT | |
| | | AAUGCCGUGUUUAUCUCGUCA | | | | | |
| | | ACUUGUUGGCGAGAUUUUU | | | | | |
| JHI3SA007 | S. aureus | GAACCUGUAUGAGUUUAUACA | 83 | GAACCUGUAUG | 175 | AACCTGTATGAG | 267 |
| | Cas9 | UGGUUUUAGUACUCUGGAAAC | | AGUUUAUACAUG | | TTTATACATGTG | |
| | | AGAAUCUACUAAAACAAGGCAA | | | | GGAT | |
| | | AAUGCCGUGUUUAUCUCGUCA | | | | | |
| | | ACUUGUUGGCGAGAUUUUU | | | | | |
| JHI3SA008 | S. aureus | GUAUAUCAAUCAUGUAUCUUC | 84 | GUAUAUCAAUCA | 176 | ATTCCTGTGAAG | 268 |
| | Cas9 | ACGUUUUAGUACUCUGGAAAC | | UGUAUCUUCAC | | ATACATGATTGA | |
| | | AGAAUCUACUAAAACAAGGCAA | | | | TATA | |
| | | AAUGCCGUGUUUAUCUCGUCA | | | | | |
| | | ACUUGUUGGCGAGAUUUUU | | | | | |
| JHI3SA011 | S. aureus | GUGGGGGCUUAAACUCUACUU | 85 | GUGGGGGCUUA | 177 | ATTCTAAAAAGT | 269 |
| | Cas9 | UUGUUUUAGUACUCUGGAAAC | | AACUCUACUUUU | | AGAGTTTAAGCC | |
| | | AGAAUCUACUAAAACAAGGCAA | | | | CCCA | |
| | | AAUGCCGUGUUUAUCUCGUCA | | | | | |
| | | ACUUGUUGGCGAGAUUUUU | | | | | |
| JHI3SA012 | S. aureus | GCUUAGAUUGCUAUUCUAAAA | 86 | GCUUAGAUUGC | 178 | CTTAGATTGCTA | 270 |
| | Cas9 | AGGUUUUAGUACUCUGGAAAC | | UAUUCUAAAAAG | | TTCTAAAAAGTA | |
| | | AGAAUCUACUAAAACAAGGCAA | | | | GAGT | |
| | | AAUGCCGUGUUUAUCUCGUCA | | | | | |
| | | ACUUGUUGGCGAGAUUUUU | | | | | |
| JHI3SA013 | S. aureus | GAGAGACGAAGGCUAUGAUUU | 87 | GAGAGACGAAG | 179 | ATTCTGCTAAAT | 271 |
| | Cas9 | AGGUUUUAGUACUCUGGAAAC | | GCUAUGAUUUA | | CATAGCCTTCGT | |
| | | AGAAUCUACUAAAACAAGGCAA | | G | | CTCT | |
| | | AAUGCCGUGUUUAUCUCGUCA | | | | | |
| | | ACUUGUUGGCGAGAUUUUU | | | | | |
| JHI3SA015 | S. aureus | GACAGGAGGCAGGCUGGGGU | 88 | GACAGGAGGCA | 180 | ATCCTATCTACC | 272 |
| | Cas9 | AGAGUUUUAGUACUCUGGAAA | | GGCUGGGGUAG | | CCAGCCTGCCTC | |
| | | CAGAAUCUACUAAAACAAGGCA | | A | | CTGT | |
| | | AAAUGCCGUGUUUAUCUCGUC | | | | | |
| | | AACUUGUUGGCGAGAUUUUU | | | | | |
| JHI3SA016 | S. aureus | GUUAAGUGUUACAGGAGGCAG | 89 | GUUAAGUGUUA | 181 | ACCCCAGCCTG | 273 |
| | Cas9 | GCGUUUUAGUACUCUGGAAAC | | CAGGAGGCAGG | | CCTCCTGTAACA | |
| | | AGAAUCUACUAAAACAAGGCAA | | C | | CTTAA | |
| | | AAUGCCGUGUUUAUCUCGUCA | | | | | |
| | | ACUUGUUGGCGAGAUUUUU | | | | | |
| JHI3SA017 | S. aureus | GAUCUGACAAUAUAUACCGAG | 90 | GAUCUGACAAUA | 182 | ACTCTTTTCTCG | 274 |
| | Cas9 | AAGUUUUAGUACUCUGGAAAC | | UAUACCGAGAA | | GTATATATTGTC | |
| | | AGAAUCUACUAAAACAAGGCAA | | | | AGAT | |
| | | AAUGCCGUGUUUAUCUCGUCA | | | | | |
| | | ACUUGUUGGCGAGAUUUUU | | | | | |
| JHI3SA018 | S. aureus | GCGGUAUAUAUUGUCAGAUAU | 91 | GCGGUAUAUAU | 183 | CGGTATATATTG | 275 |
| | Cas9 | CUGUUUUAGUACUCUGGAAAC | | UGUCAGAUAUCU | | TCAGATATCTCT | |
| | | AGAAUCUACUAAAACAAGGCAA | | | | GGGT | |
| | | AAUGCCGUGUUUAUCUCGUCA | | | | | |
| | | ACUUGUUGGCGAGAUUUUU | | | | | |
| JHI3SA019 | S. aureus | GAUCAAGGUUUCUGGCAGACA | 92 | GAUCAAGGUUU | 184 | ACCCCAACTGTC | 276 |
| | Cas9 | GUGUUUUAGUACUCUGGAAAC | | CUGGCAGACAG | | TGCCAGAAACCT | |
| | | AGAAUCUACUAAAACAAGGCAA | | U | | TGAT | |
| | | AAUGCCGUGUUUAUCUCGUCA | | | | | |
| | | ACUUGUUGGCGAGAUUUUU | | | | | |

Thus, the disclosure provides nucleic acids for correcting single and multiple exon duplications of the DMD gene resulting from a mutation involving, surrounding, or affecting a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3,3-4, 3-5, 3-6, 3-7, 3-8,3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene. The DMD gene is the largest known gene in humans. It is 2.4 million base-pairs in size, comprises 79 exons and takes over 16 hours to be transcribed and cotranscriptionally spliced. The result of this Cas9 gene editing process allows the body to dystrophin. In some aspects, the dystrophin is a full-length dystrophin, or a functional form of dystrophin which prevents, ameliorates, or treats a muscular dystrophy which would result or results from the mutation in the DMD gene. In some aspects, the dystrophin is a shorter, usable dystrophin which, in some aspects, makes the effects of such DMD mutation less severe.

### Dystrophin and Duchenne Muscular Dystrophy

The disclosure provides products, methods and uses for treating a muscular dystrophy resulting from a mutation in the DMD gene. Such muscular dystrophies include, but are not limited to, Duchenne muscular dystrophy (DMD), Becker muscular dystrophy (BMD), and Intermediate muscular dystrophy (IMD). DMD is an X-linked genetic disorder caused by myriad mutations within the DMD gene which contains a total of 79 exons and codes for the 427 kDa muscle isoform of the dystrophin protein (Flanigan, Neurol Clin 32, 671-688, viii, doi:10.1016/j.ncl.2014.05.002 (2014)). The DMD gene encodes the dystrophin protein, which is one of the longest human genes known. Dystrophin is a structural protein which serves to reinforce the plasma membrane via a connection between cytoskeletal actin filaments and the dystroglycan complex (DGC) (Gao et al., Compr Physiol 5, 1223-1239, doi:10.1002/cphy.c140048 (2015)). As such, dystrophin has several key domains including an N-terminal actin binding domain, a central rod domain comprised of spectrin-like repeats with a second actin binding domain, and a C-terminal domain that directly interacts with the DGC (Gao et al., supra). Dystrophin acts as a shock-absorber during normal muscle contraction and is required to prevent muscle damage and degeneration during normal activity. In the absence of dystrophin, muscle degeneration leads to weakness which eventually progresses to a loss of ambulation in the early teens. Once in a wheelchair, patients have steep declines in cardiac and respiratory function (due to the involvement of the heart and diaphragm, respectively) which are the primary causes of the early mortality characteristic of DMD.

The DMD gene, the gene encoding the dystrophin protein, has a diverse mutational profile, due in part to the size of the gene (Bladen et al., Hum Mutat 36, 395-402, doi:10.1002/humu.22758 (2015)). Exonic duplications occur when a portion of the gene is duplicated and placed directly adjacent to the original gene fragment (Bladen et al. supra). Exonic deletions are when a portion of the gene containing one or more exons is fully excised from the gene (Bladen et al. supra). Both exonic deletions and duplications usually result in frameshift mutations that generally lead to loss of functional dystrophin protein. Other DMD mutations consist of subexonic insertions and deletions (indels) that also generally result in frameshift mutations (Bladen et al. supra). Other DMD mutations consist of mutations that affect the splice sites of certain exons (Bladen et al. supra). Still other DMD mutations consist of variable and highly specific mutations throughout the intronic regions of the DMD gene (Bladen et al. supra). Despite this extensive mutational profile, gene editing has shown great potential in correcting many of the types of mutations described above.

### CRISPR-Cas9 Gene Editing

Clustered Regularly Interspaced Short Palindromic Repeats and the associated protein 9 (“CRISPR-associated protein 9” or “CRISPR-Cas9”) is an adaptive immune system found in bacteria that utilizes an RNA-programmable endonuclease to protect bacteria against viral invaders. This system, which consists of a guide RNA (gRNA) and a Cas9 endonuclease protein, has been repurposed to make precise double stranded breaks (DSBs) at a site complementary to the gRNA and near a short recognition sequence known as a protospacer adjacent motif (PAM) site. Cas9 (CRISPR associated protein 9, formerly called Cas5, Csn1, or Csx12) is a 160 kilo Dalton protein which plays a vital role in the immunological defense of certain bacteria against DNA viruses and plasmids and which is heavily utilized in genetic engineering applications. Cas9 is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms (Zhang et al. (2014) Human Molecular Genetics. 23 (R1): R40-6. doi:10.1093/hmg/ddu125. PMID 24651067). This editing process has a wide variety of applications including basic biological research, development of biotechnology products, and treatment of diseases.

The disclosure utilizes CRISPR-Cas9 in the gene editing complex, methods and uses disclosed herein. The disclosure included the use of all species, homologs, orthologs, and variants of Cas9, including functional fragments thereof. Thus, as used herein, the term “Cas9”, unless expressly stated otherwise, includes all Cas9 species, homologs, orthogs, variants, including engineered Cas9 variants (e.g., Liu et al., Nat Commun 11, 3576 (2020); WO 2014/191521) and split-Cas9 (e.g., WO 2016/112242; WO 2017/197238), and functional fragments thereof. As used herein, the term “Cas9”, unless expressly stated otherwise, is any Cas9 species, homolog, ortholog, variant, engineered variant, including split-Cas9, mammalian codon-optimized Cas9, or a functional fragment thereof.

There are several different species and homologs of the Cas9 protein from different bacteria which have differences in size and PAM recognition sequence. The most well characterized variant is Cas9 from Streptococcus pyogenes (SpCas9) which is encoded by 1,371 amino acids and has a PAM recognition sequence of 5′-NGG-3′ (Jinek et al., Science 337, 816-821, doi:10.1126/science.1225829 (2012); Ran et al., Nat Protoc 8, 2281-2308, doi:10.1038/nprot.2013.143 (2013); Zhang et al., Physiol Rev 98, 1205-1240, doi:10.1152/physrev.00046.2017 (2018)). A less commonly used Cas protein is from Staphylococcus aureus (SaCas9) which, in contrast to SpCas9, is encoded by 1,053 amino acids and has a PAM recognition sequence of 5′-NNGRRT-3′ (SEQ ID NO: 279) (Ran et al., Nature 520, 186-191, doi:10.1038/nature14299 (2015)). The use of the smaller SaCas9 protein is preferable, in some aspects, in virally delivered gene therapies on account of the limited cargo space (˜5 kb) associated with viral vectors such as the Adeno-Associated Virus (AAV) (Grieger et al., J Virol 79, 9933-9944, doi:10.1128/JVI.79.15.9933-9944.2005 (2005)). Nevertheless, the disclosure includes the use of all various species, homologs, orthologs, and variants of Cas9, as well as functional fragments thereof, and is not limited to the particular Cas9 exemplified herein. In various exemplary aspects of the disclosure, Staphylococcus aureus (SaCas9) and Campylobacter jejuni Cas9 (CjCas9) are provided. The disclosure is not limited to these particular species of Cas9. In some aspects, the Cas9 is mammalian codon optimized. In some aspects, e.g., the SaCas9 is described by Tan et al. (PNAS Oct. 15, 2019 116 (42) 20969-20976; https://doi.org/10.1073/pnas.1906843116). In some aspects, the Campylobacter jejuni Cas9 is commercially available, e.g., PX404 from Addgene (Cat. No. 68338, https://www.addgene.org/68338/sequences/). In some aspects, the SpCas9 is described in the literature (UniProtKB—Q1JH43 (Q1JH43_STRPD).

In exemplary aspects, the disclosure provides Cas9 coding sequences. In exemplary aspects, Cas9 is encoded by the nucleic acid comprising the nucleotide sequence set out in SEQ ID NO: 277 or 278 (Table 2), a variant thereof comprising at least about 70%, about 75%, about 80%, about 85%, about 90%, 91%, 92%, 93%, 94%, 95%, 96%, 97%, 98%, or 99% identity to the sequence set out in SEQ ID NO: 277 or 278, or a functional fragment thereof. In exemplary aspects, the disclosure provides the nucleotide sequences encoding S. aureus Cas9 (SEQ ID NO: 277) and C. jejuni Cas9 (SEQ ID NO: 278) as set out in Table 2.

TABLE 2
Exemplary Cas9 Coding Sequences.

| SEQ | | |
|---|---|---|
| ID | | |
| NO: | Species | Cas9 Coding Sequence |
| 277 | S. aureus | ATGGCCCCAAAGAAGAAGCGGAAGGTCGGTATCCACGGAGTCCCA [symbol] |
| | | CAGCCAAGCGGAACTACATCCTGGGCCTGGACATCGGCATCACCAG |
| | | CGTGGGCTACGGCATCATCGACTACGAGACACGGGACGTGATCGAT |
| | | GCCGGCGTGCGGCTGTTCAAAGAGGCCAACGTGGAAAACAACGAG |
| | | GGCAGGCGGAGCAAGAGAGGCGCCAGAAGGCTGAAGCGGCGGAG |
| | | GCGGCATAGAATCCAGAGAGTGAAGAAGCTGCTGTTCGACTACAAC |
| | | CTGCTGACCGACCACAGCGAGCTGAGCGGCATCAACCCCTACGAG [symbol] |
| | | CCAGAGTGAAGGGCCTGAGCCAGAAGCTGAGCGAGGAAGAGTTCT [symbol] |
| | | TGCCGCCCTGCTGCACCTGGCCAAGAGAAGAGGCGTGCACAACGT [symbol] |
| | | AACGAGGTGGAAGAGGACACCGGCAACGAGCTGTCCACCAAAGAG [symbol] |
| | | AGATCAGCCGGAACAGCAAGGCCCTGGAAGAGAAATACGTGGCCGA |
| | | ACTGCAGCTGGAACGGCTGAAGAAAGACGGCGAAGTGCGGGGCAG |
| | | CATCAACAGATTCAAGACCAGCGACTACGTGAAAGAAGCCAAACAG [symbol] |
| | | TGCTGAAGGTGCAGAAGGCCTACCACCAGCTGGACCAGAGCTTCAT |
| | | CGACACCTACATCGACCTGCTGGAAACCCGGCGGACCTACTATGAG |
| | | GGACCTGGCGAGGGCAGCCCCTTCGGCTGGAAGGACATCAAAGAA [symbol] |
| | | GGTACGAGATGCTGATGGGCCACTGCACCTACTTCCCCGAGGAACT |
| | | GCGGAGCGTGAAGTACGCCTACAACGCCGACCTGTACAACGCCCT [symbol] |
| | | AACGACCTGAACAATCTCGTGATCACCAGGGACGAGAACGAGAAGC |
| | | TGGAATATTACGAGAAGTTCCAGATCATCGAGAACGTGTTCAAGCAG |
| | | AAGAAGAAGCCCACCCTGAAGCAGATCGCCAAAGAAATCCTCGTGA |
| | | ACGAAGAGGATATTAAGGGCTACAGAGTGACCAGCACCGGCAAGC [symbol] |
| | | CGAGTTCACCAACCTGAAGGTGTACCACGACATCAAGGACATTACC [symbol] |
| | | CCCGGAAAGAGATTATTGAGAACGCCGAGCTGCTGGATCAGATTGC |
| | | CAAGATCCTGACCATCTACCAGAGCAGCGAGGACATCCAGGAAGAA |
| | | CTGACCAATCTGAACTCCGAGCTGACCCAGGAAGAGATCGAGCAGA |
| | | TCTCTAATCTGAAGGGCTATACCGGCACCCACAACCTGAGCCTGAA [symbol] |
| | | GCCATCAACCTGATCCTGGACGAGCTGTGGCACACCAACGACAACC |
| | | AGATCGCTATCTTCAACCGGCTGAAGCTGGTGCCCAAGAAGGTGGA |
| | | CCTGTCCCAGCAGAAAGAGATCCCCACCACCCTGGTGGACGACTTC |
| | | ATCCTGAGCCCCGTCGTGAAGAGAAGCTTCATCCAGAGCATCAAAG |
| | | GATCAACGCCATCATCAAGAAGTACGGCCTGCCCAACGACATCATTA |
| | | TCGAGCTGGCCCGCGAGAAGAACTCCAAGGACGCCCAGAAAATGAT |
| | | CAACGAGATGCAGAAGCGGAACCGGCAGACCAACGAGCGGATCGA |
| | | GGAAATCATCCGGACCACCGGCAAAGAGAACGCCAAGTACCTGATC |
| | | GAGAAGATCAAGCTGCACGACATGCAGGAAGGCAAGTGCCTGTACA |
| | | GCCTGGAAGCCATCCCTCTGGAAGATCTGCTGAACAACCCCTTCAA [symbol] |
| | | TATGAGGTGGACCACATCATCCCCAGAAGCGTGTCCTTCGACAACA [symbol] |
| | | CTTCAACAACAAGGTGCTCGTGAAGCAGGAAGAAAACAGCAAGAAG |
| | | GGCAACCGGACCCCATTCCAGTACCTGAGCAGCAGCGACAGCAAGA |
| | | TCAGCTACGAAACCTTCAAGAAGCACATCCTGAATCTGGCCAAGGG [symbol] |
| | | AAGGGCAGAATCAGCAAGACCAAGAAAGAGTATCTGCTGGAAGAAC |
| | | GGGACATCAACAGGTTCTCCGTGCAGAAAGACTTCATCAACCGGAA [symbol] |
| | | CTGGTGGATACCAGATACGCCACCAGAGGCCTGATGAACCTGCTGC |
| | | GGAGCTACTTCAGAGTGAACAACCTGGACGTGAAAGTGAAGTCCAT [symbol] |
| | | AATGGCGGCTTCACCAGCTTTCTGCGGCGGAAGTGGAAGTTTAAGA |
| | | AGAGCGGAACAAGGGGTACAAGCACCACGCCGAGGACGCCCTGAT |
| | | CATTGCCAACGCCGATTTCATCTTCAAAGAGTGGAAGAAACTGGACA |
| | | AGGCCAAAAAAGTGATGGAAAACCAGATGTTCGAGGAAAAGCAGGC |
| | | CGAGAGCATGCCCGAGATCGAAACCGAGCAGGAGTACAAAGAGAT [symbol] |
| | | TTCATCACCCCCCACCAGATCAAGCACATTAAGGACTTCAAGGACTA |
| | | CAAGTACAGCCACCGGGTGGACAAGAAGCCTAATAGAGAGCTGATT |
| | | AACGACACCCTGTACTCCACCCGGAAGGACGACAAGGGCAACACC [symbol] |
| | | TGATCGTGAACAATCTGAACGGCCTGTACGACAAGGACAATGACAA [symbol] |
| | | CTGAAAAAGCTGATCAACAAGAGCCCCGAAAAGCTGCTGATGTACCA |
| | | CCACGACCCCCAGACCTACCAGAAACTGAAGCTGATTATGGAACAG [symbol] |
| | | ACGGCGACGAGAAGAATCCCCTGTACAAGTACTACGAGGAAACCGG |
| | | GAACTACCTGACCAAGTACTCCAAAAAGGACAACGGCCCCGTGATC [symbol] |
| | | AGAAGATTAAGTATTACGGCAACAAACTGAACGCCCATCTGGACATC |
| | | ACCGACGACTACCCCAACAGCAGAAACAAGGTCGTGAAGCTGTCCC |
| | | TGAAGCCCTACAGATTCGACGTGTACCTGGACAATGGCGTGTACAA [symbol] |
| | | TTCGTGACCGTGAAGAATCTGGATGTGATCAAAAAAGAAAACTACTA |
| | | CGAAGTGAATAGCAAGTGCTATGAGGAAGCTAAGAAGCTGAAGAAG |
| | | ATCAGCAACCAGGCCGAGTTTATCGCCTCCTTCTACAACAACGATCT |
| | | GATCAAGATCAACGGCGAGCTGTATAGAGTGATCGGCGTGAACAAC |
| | | GACCTGCTGAACCGGATCGAAGTGAACATGATCGACATCACCTACC |
| | | GCGAGTACCTGGAAAACATGAACGACAAGAGGCCCCCCAGGATCAT |
| | | TAAGACAATCGCCTCCAAGACCCAGAGCATTAAGAAGTACAGCACA [symbol] |
| | | ACATTCTGGGCAACCTGTATGAAGTGAAATCTAAGAAGCACCCTCAG |
| | | ATCATCAAAAAGGGCAAAAGGCCGGCGGCCACGAAAAAGGCCGGC [symbol] |
| | | AGGCAAAAAAGAAAAAGTAA |
| 278 | C. jejuni | ATGGCCCCAAAGAAGAAGCGGAAGGTCGGTGCTCGCATACTCGCTT |
| | | TTGATATTGGAATTTCATCCATAGGATGGGCATTTTCAGAAAATGATG |
| | | AACTTAAAGATTGTGGAGTCAGGATTTTCACAAAAGTAGAGAATCCC [symbol] |
| | | AAACAGGGGAAAGCCTTGCTCTCCCAAGGAGACTGGCGCGATCCG [symbol] |
| | | AAGGAAACGACTTGCTAGGCGCAAAGCAAGGTTGAATCATCTTAAA [symbol] |
| | | ATCTCATTGCTAATGAATTTAAACTCAATTATGAAGATTACCAAAGTTT |
| | | TGATGAATCTTTGGCTAAAGCGTATAAAGGTAGTCTCATTTCCCCATA |
| | | TGAACTCCGTTTTCGCGCATTGAATGAACTTCTCTCTAAACAAGATTT |
| | | TGCTCGTGTCATTCTTCACATTGCAAAACGTCGCGGTTATGATGATA [symbol] |
| | | TAAGAATTCAGATGATAAGGAAAAGGGAGCGATTCTCAAAGCTATTA |
| | | AACAAAATGAGGAGAAATTGGCTAACTATCAATCTGTCGGAGAATAT |
| | | CTCTATAAGGAATATTTCCAAAAGTTTAAGGAAAATTCCAAGGAATTT |
| | | ACAAATGTGCGAAATAAGAAGGAGTCCTATGAAAGGTGCATTGCTCA |
| | | ATCCTTTCTCAAAGACGAACTCAAACTCATCTTTAAGAAACAAAGGGA |
| | | ATTTGGGTTTAGTTTTAGTAAGAAGTTTGAAGAGGAAGTATTGTCAGT |
| | | GGCTTTCTATAAACGGGCTCTCAAGGACTTTTCTCATCTGGTCGGAA |
| | | ATTGTTCTTTCTTTACGGATGAAAAGCGGGCACCGAAGAATTCACCA |
| | | CTCGCGTTTATGTTTGTCGCACTCACTCGCATTATTAATCTCCTCAAT |
| | | AACCTTAAGAATACAGAAGGAATTCTTTATACAAAAGATGATCTCAAT |
| | | GCGCTGCTTAATGAAGTTTTGAAGAATGGAACTCTTACTTATAAACAA |
| | | ACAAAGAAGTTGCTTGGGTTGTCAGATGATTATGAATTCAAAGGAGA |
| | | GAAAGGTACTTATTTTATCGAGTTTAAGAAATATAAAGAGTTTATTAAA |
| | | GCACTCGGAGAACATAATCTCTCCCAAGACGACCTTAATGAAATTGC |
| | | AAAAGATATTACACTCATTAAAGATGAAATAAAACTGAAGAAAGCACT |
| | | TGCAAAATATGATCTGAATCAAAATCAAATCGATTCACTTTCTAAATT [symbol] |
| | | GAGTTTAAAGACCATTTGAATATTTCTTTCAAAGCACTTAAATTGGTC [symbol] |
| | | CACCACTCATGCTTGAGGGGAAGAAATACGATGAAGCCTGTAATGA [symbol] |
| | | CTTAATTTGAAAGTCGCTATTAATGAAGATAAGAAGGATTTTCTTCA |
| | | GCTTTTAATGAAACCTATTATAAAGATGAGGTTACGAATCCGGTTGT [symbol] |
| | | TTGCGAGCAATTAAGGAATATAGGAAAGTACTCAACGCTTTGCTCAA |
| | | GAAGTATGGTAAAGTACATAAAATTAATATTGAACTTGCCCGCGAGG |
| | | CGGTAAGAATCATTCACAACGGGCTAAAATTGAAAAGGAGCAAAATG |
| | | AAAATTATAAAGCGAAGAAAGACGCAGAACTCGAGTGTGAAAAGTTG |
| | | GGCCTCAAAATTAATTCCAAGAATATACTCAAGCTTCGGCTGTTTAA [symbol] |
| | | GAACAAAAGGAGTTTTGTGCATATAGTGGAGAGAAAATCAAAATCTC |
| | | CGATCTTCAAGACGAAAAGATGCTGGAAATTGACCATATTTATCCATA |
| | | TTCTAGGTCTTTTGATGATAGTTATATGAATAAAGTCCTTGTATTTACA |
| | | AAACAAAACCAGGAGAAACTTAACCAAACTCCCTTTGAGGCTTTTGG |
| | | GAATGATTCCGCAAAATGGCAAAAGATTGAAGTATTGGCTAAGAATC |
| | | TCCCGACCAAGAAACAGAAACGAATTTTGGATAAGAACTATAAAGAT [symbol] |
| | | AAGAGCAGAAGAATTTTAAAGATAGAAATCTCAATGATACTCGATACA |
| | | TTGCTCGCCTTGTCTTGAATTATACCAAAGACTATTTGGACTTTCTCC |
| | | CCCTCTCAGATGATGAAAATACCAAATTGAATGACACTCAAAAGGGA |
| | | TCAAAAGTCCATGTTGAGGCCAAAAGTGGGATGCTCACTTCCGCACT |
| | | CCGCCATACGTGGGGATTTTCCGCAAAAGACAGGAATAATCACCTG [symbol] |
| | | ATCATGCTATAGATGCTGTTATAATAGCATATGCAAATAATTCCATTG |
| | | CAAAGCCTTTTCTGATTTTAAGAAGGAACAGGAAAGTAATTCTGCAGA |
| | | ATTGTATGCTAAGAAGATTTCCGAACTCGATTATAAGAATAAAAGAAA |
| | | ATTCTTTGAACCATTTAGTGGGTTTCGGCAAAAGGTCTTGGACAAAA [symbol] |
| | | TGATGAAATATTTGTCAGCAAACCAGAAAGGAAGAAACCATCCGGAG |
| | | CGCTTCATGAAGAGACTTTTCGGAAGGAAGAGGAATTTTATCAAAGC |
| | | TATGGCGGAAAAGAGGGAGTTCTTAAAGCGTTGGAGCTCGGTAAAA |
| | | ACGGAAGGTCAATGGTAAAATAGTTAAGAACGGGGATATGTTTAGG [symbol] |
| | | TTGATATATTTAAACATAAGAAAACAAATAAATTTTATGTGTTCCCAT |
| | | TTATACTATGGACTTTGCATTGAAAGTCTTGCCGAATAAAGCGGTCG [symbol] |
| | | TAGGTCCAAGAAAGGAGAGATTAAAGACTGGATATTGATGGATGAAA |
| | | ACTACGAATTTTGCTTTTCCTTGTATAAAGATAGCCTGATTTTGATACA |
| | | AACCAAAGATATGCAGGAACCAGAATTTGTTTATTATAATGCGTTTAC |
| | | AAGTAGTACTGTCAGCCTTATTGTCTCCAAACATGACAATAAATTTGA |
| | | AACCCTCAGTAAGAATCAGAAAATTTTGTTTAAGAATGCGAATGAGAA |
| | | AGAGGTTATTGCAAAATCCATTGGAATTCAAAATTTGAAGGTATTCGA |
| | | GAAGTATATTGTCAGCGCGCTCGGAGAGGTTACTAAAGCTGAATTC [symbol] |
| | | GCCAACGCGAAGATTTCAAGAAAAAAAGGCCGGCGGCCACGAAAAA |
| | | GGCCGGCCAGGCAAAAAAGAAAAAGTGA |

[symbol]

indicates data missing or illegible when filed

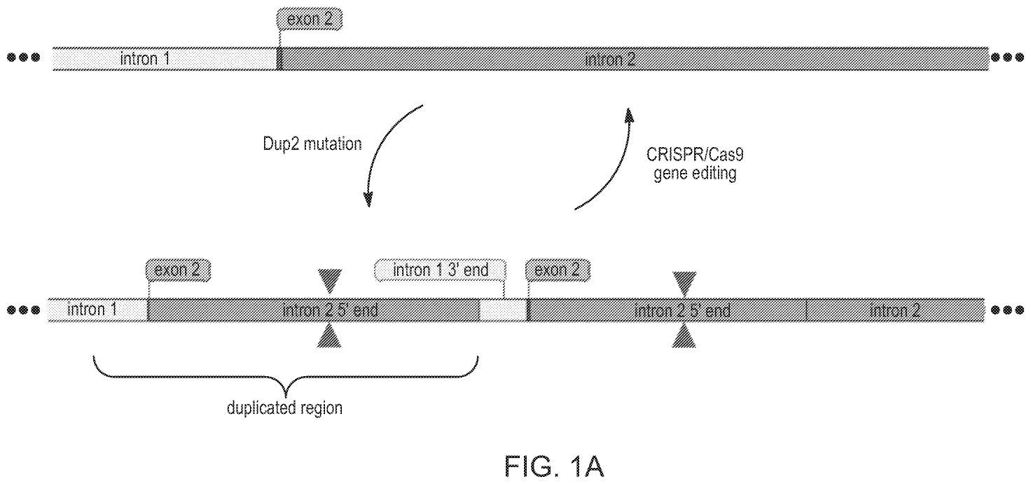

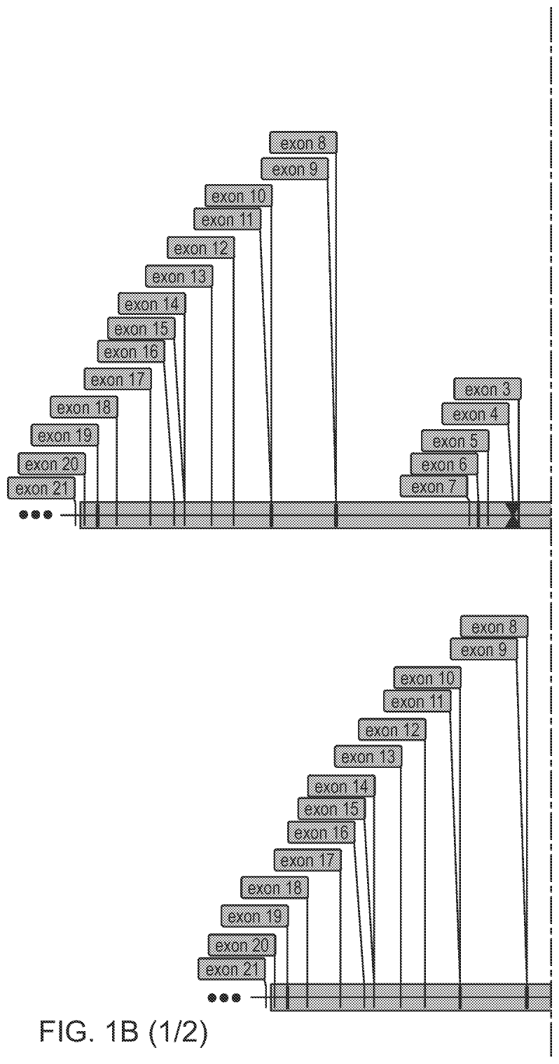

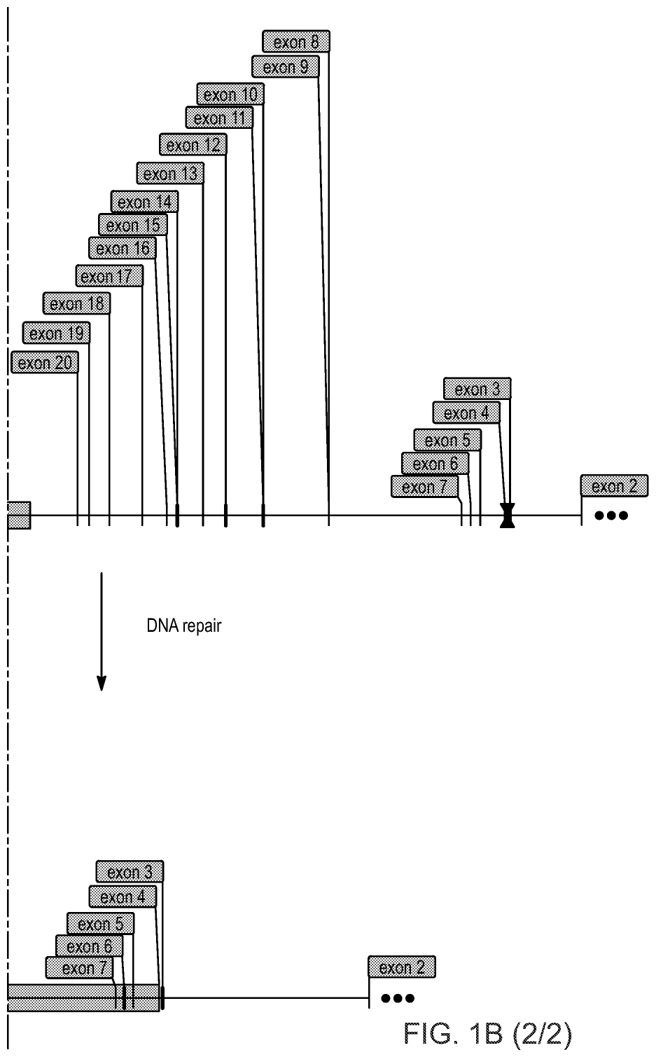

CRISPR-Cas9 somatic cell gene editing has enormous potential to correct DMD mutations and provide meaningful benefits to patients. While dystrophinopathies can be caused by a myriad of mutations of the DMD gene, exon duplications are among the most common affecting many dystrophinopathy patients. The disclosure provides an approach to correct exon duplications wherein a single guide-RNA (gRNA) is used with Cas9 to generate two cuts that excise the duplicated region of DNA and result in reversion to the normal coding sequence (FIG. 1A-B). Rather than reframing to generate a functional mutant isoform as is achieved with most contemporary exon deletion and skipping approaches, reversion of the DMD gene to the normal coding sequence (CDS) and restoration of full-length dystrophin expression, as carried out with the products, methods, and uses described herein, provide the most robust and long-term benefits to subjects with a dystrophinopathy or muscular dystrophy resulting from one or more DMD gene mutations.

In some aspects, the nucleic acid encoding Cas9 is inserted into a mammalian expression vector, including a viral vector for expression in cells. In some aspects, the nucleic acid encoding mammalian gRNA for Cas9 is cloned into a mammalian expression vector, including a viral vector for expression in cells.

In some aspects, the DNA encoding the gRNA and/or the Cas9 are under expression of a promoter. In some aspects, the promoter is a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a CK8 promoter, a CK8e promoter, an SPC5-12 promoter, or a desmin promoter.

In some aspects, the promoter is a U6 promoter. The endogenous U6 promoter normally controls expression of the U6 RNA, a small nuclear RNA (snRNA) involved in splicing, and has been well-characterized [Kunkel et al., Nature. 322(6074):73-7 (1986); Kunkel et al., Genes Dev. 2(2):196-204 (1988); Paule et al., Nucleic Acids Res. 28(6):1283-98 (2000)]. In some aspects, the U6 promoter is used to control vector-based expression of shRNA molecules in mammalian cells [Paddison et al., Proc. Natl. Acad. Sci. USA 99(3):1443-8 (2002); Paul et al., Nat. Biotechnol. 20(5):505-8 (2002)] because (1) the promoter is recognized by RNA polymerase Ill (poly Ill) and controls high-level, constitutive expression of shRNA; and (2) the promoter is active in most mammalian cell types. In some aspects, the promoter is a type III Pol Ill promoter in that all elements required to control expression of the shRNA are located upstream of the transcription start site (Paule et al., Nucleic Acids Res. 28(6):1283-98 (2000)). The disclosure includes both murine and human U6 promoters. The shRNA containing the sense and antisense sequences from a target gene connected by a loop is transported from the nucleus into the cytoplasm where Dicer processes it into small/short interfering RNAs (siRNAs). In some aspects, the nucleotide sequence encoding mammalian gRNA for Cas9 is under control of a U6 promoter. In some aspects, the nucleotide sequence encoding Cas9 is under control of a MHCK7 promoter.

Embodiments of the disclosure utilize vectors (for example, viral vectors, such as adeno-associated virus (AAV), adenovirus, retrovirus, lentivirus, equine-associated virus, alphavirus, pox viruses, herpes virus, polio virus, sindbis virus and vaccinia viruses) to deliver the nucleic acids disclosed herein, for example, nucleic acids comprising polynucleotides encoding DMD gRNAs and Cas9 enzymes disclosed herein. In some aspects, a nucleotide sequence encoding a DMD-targeted gRNA and a nucleotide sequence encoding Cas9 are cloned individually into separate vectors. In some aspects, a nucleotide sequence encoding a DMD-targeted gRNA and a nucleotide sequence encoding Cas9 are cloned into the same vector. Thus, in some aspects the disclosure includes vectors comprising one or more of the nucleotide sequences described herein above in the disclosure. In some aspects, the vectors are AAV vectors. In some aspects, the vectors are single stranded AAV (ssAAV) vectors. In some aspects the AAV is recombinant AAV (rAAV). In some aspects, the rAAV lack rep and cap genes. In some aspects, rAAV are self-complementary (sc)AAV. In various aspects throughout the disclosure, AAV is rAAV, scAAV, or ssAAV.

In some aspects, the disclosure utilizes adeno-associated virus (AAV) to deliver nucleic acids encoding the gRNA and/or nucleic acids encoding Cas9. AAV is a replication-deficient parvovirus, the single-stranded DNA genome of which is about 4.7 kb in length including 145 nucleotide inverted terminal repeat (ITRs). There are multiple serotypes of AAV. In some aspects, the AAV is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et al., J. Virol., 45: 555-564 {1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively (see also U.S. Pat. Nos. 7,282,199 and 7,790,449 relating to AAV8); the AAV9 genome is provided in Gao et al., J. Virol., 78: 6381-6388 (2004); the AAV10 genome is provided in Mol. Ther., 13(1): 67-76 (2006); and the AAV11 genome is provided in Virology, 330(2): 375-383 (2004). Information regarding MyoAAV 1A is provided by Tabebordbar et al. (Cell 184(19): 4919-38 (2021)). Information regarding AAVMYO is provided by Weinmann et al. (Nature Communications 11: 5432 (2020); doi.org/10.1038/s41467-020-19230). The genomes of AAV12, AAV13, AAVanc80, AAVrh.74, AAVrh.8, AAVrh.10, and AAV-B1 also are known in the art. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may be lyophilized and AAV-infected cells are not resistant to superinfection.

Recombinant AAV genomes of the disclosure comprise one or more AAV ITRs flanking at least one DMD-targeted polynucleotide construct. In some embodiments, the polynucleotide is a gRNA or a polynucleotide encoding the gRNA. In some aspects, the gRNA is administered with other polynucleotide constructs targeting DMD. Thus, in some aspects, the polynucleotide encoding the DMD gRNA is administered with a polynucleotide encoding the DMD donor sequence. In various aspects, the gRNA is expressed under various promoters including, but not limited to, such promoters as a U6 promoter, a U7 promoter, a T7 promoter, a tRNA promoter, an H1 promoter, an EF1-alpha promoter, a minimal EF1-alpha promoter, an unc45b promoter, a CK1 promoter, a CK6 promoter, a CK7 promoter, a miniCMV promoter, a CMV promoter, a muscle creatine kinase (MCK) promoter, an alpha-myosin heavy chain enhancer-/MCK enhancer-promoter (MHCK7), a tMCK promoter, a minimal MCK promoter, a CK8 promoter, a CK8e promoter, an SPC5-12 promoter, or a desmin promoter Specifically, this strategy is used, in various aspects, to achieve more efficient expression of the same gRNA in multiple copies in a single backbone. AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. As set out herein above, the nucleotide sequences of the genomes of various AAV serotypes are known in the art.

In some aspects, the disclosure provides a recombinant adeno-associated virus (rAAV) comprising a genome comprising at least one of the nucleic acid molecules disclosed or described herein. In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAVanc80, rAAV rh.74, rAAVrh.8, rAAVrh.10, MyoAAV 1A, AAVMYO, or rAAV-B1. In some aspects, the disclosure provides an rAAV, wherein the genome of the rAAV lacks AAV rep and cap DNA. In some aspects, the disclosure provides an rAAV, wherein the rAAV further comprises an AAV1 capsid, an AAV2 capsid, an AAV3 capsid, an AAV4 capsid, an AAV5 capsid, an AAV6 capsid, an AAV7 capsid, an AAV8 capsid, an AAV9 capsid, an AAV10 capsid, an AAV11 capsid, an AAV12 capsid, an AAV13 capsid, an rAAVanc80 capsid, an AAVrh.74 capsid, an rAAVrh.8 capsid, an rAAVrh.10 capsid, a MyoAAV 1A capsid, a AAVMYO capsid, or an rAAV-B1 capsid.

In some aspects, the disclosure provides a scAAV comprising a genome comprising at least one of the nucleic acid molecules disclosed or described herein. In some aspects, the scAAV is scAAV1, scAAV2, scAAV3, scAAV4, scAAV5, scAAV6, scAAV7, scAAV8, scAAV9, scAAV10, scAAV11, scAAV12, scAAV13, scAAVanc80, scAAV rh.74, scAAVrh.8, scAAVrh.10, scMyoAAV 1A, scAAVMYO, or scAAV-B1.

DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. In some aspects, AAV DNA in the rAAV genomes is from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

Recombinant AAV genomes of the disclosure comprise one or more AAV ITRs flanking a polynucleotide encoding, for example, one or more guide RNAs or Cas9. Embodiments of the disclosure, therefore include a rAAV genome comprising a nucleic acid comprising a nucleotide sequence set out in any of SEQ ID NOs: 1-186, or a nucleotide sequence comprising at least or about or at least about 70%, about 75%, about 80%, about 85%, about 90%, about 91%, about 92%, about 93%, about 94%, about 95%, about 96%, about 97%, about 98%, or about 99% identity to a sequence set out in any of SEQ ID NOs: 1-186.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology, 1533-539 (1992); and Muzyczka, Curr Topics in Microbial and Immunol, 158:97-129 (1992)). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988); Samulski et al., J. Virol., 63:3822-8 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine 13:1244-50 (1995); Paul et al., Human Gene Therapy 4:609-615 (1993); Clark et al., Gene Therapy 3:1124-32 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Cell transduction efficiencies of the methods of the disclosure described above and below may be at least about 60, 65, 70, 75, 80, 85, 90 or 95 percent efficient.

General principles of rAAV production are reviewed in, for example, Carter, 1992, Current Opinions in Biotechnology, 1533-539; and Muzyczka, 1992, Curr. Topics in Microbial. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment, packaging cells are stably transformed cancer cells, such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, rAAV is purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another embodiment, the disclosure includes a composition comprising rAAV comprising any of the constructs described herein. In some aspects, the disclosure includes a composition comprising the rAAV for delivering the gRNA described herein. In some aspects, the disclosure includes a composition the rAAV comprising one or more of the polynucleotide sequences encoding the gRNA described herein along with one or more polynucleotide sequences encoding Cas9. Compositions of the disclosure comprise rAAV and one or more pharmaceutically or physiologically acceptable carriers, excipients or diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

In some aspects, the disclosure includes a dual-plasmid system comprising one plasmid comprising one or more sequences encoding or comprising the gRNA; and a second plasmid comprising sequence encoding Cas9 capable of generating double-stranded DNA breaks at DNA loci determined by a gRNA spacer sequence. In some aspects, the plasmids are introduced into an AAV for delivery. In some aspects, the AAV is an rAAV, an scAAV, or an ssAAV. In some aspects, the plasmids are introduced into the cell via non-vectorized delivery.

In some other aspects, the nucleic acids are introduced into the cell via non-vectorized delivery. Thus, in an embodiment, the disclosure includes non-vectorized delivery of a nucleic acid encoding the DMD-targeting gRNA or Cas9. In some aspects, in this context, synthetic carriers able to form complexes with nucleic acids, and protect them from extra- and intracellular nucleases, are an alternative to viral vectors. In some aspects, such non-vectorized delivery includes the use of nanoparticles, extracellular vesicles, or exosomes comprising the nucleic acids of the disclosure. The disclosure also includes compositions comprising any of the constructs described herein alone or in combination.

Sterile injectable solutions are prepared by incorporating AAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of AAV to be administered in methods of the disclosure will vary depending, for example, on the particular AAV, the mode of administration, the treatment goal, the individual, and the cell type(s) being targeted, and may be determined by methods standard in the art. Titers of AAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg) (e.g., 1×10⁷ vg, 1×10⁸ vg, 1×10⁹ vg, 1×10¹⁰ vg, 1×10¹¹ vg, 1×10¹² vg, 1×10¹³ vg, and 1×10¹⁴ vg, respectively).

In an embodiment, the disclosure includes non-vectorized delivery of the nucleic acids encoding the gRNAs and/or nucleic acids encoding Cas9. In some aspects, in this context, synthetic carriers able to form complexes with nucleic acids, and protect them from extra- and intracellular nucleases, are an alternative to viral vectors. The disclosure includes such non-vectorized delivery. The disclosure also includes compositions comprising any of the constructs described herein alone or in combination.

In some aspects, the disclosure provides a method of delivering any one or more nucleic acids comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9 to a cell or to a subject in need thereof. In some aspects, the method comprises administering to the subject an AAV comprising one or more nucleotide sequences encoding (i) a DMD-targeted gRNA (e.g., a gRNA targeting a mutation involving, surrounding, or affecting a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7,3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), and (ii) a Cas9 enzyme. In some aspects, the nucleic acid encoding the Cas9 enzyme comprises the nucleotide sequence set forth in SEQ ID NO: 277 or 278, or a variant thereof comprising at least about 70% identity to the nucleotide sequence set forth in in SEQ ID NO: 277 or 278, or a functional fragment thereof. In some aspects, the method comprises administering to the subject a nucleic acid comprising a nucleotide sequence encoding (i) a gRNA, wherein at least one gRNA targets a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene, and (ii) a Cas9 enzyme or a functional fragment thereof. In some aspects, the method comprises delivering the nucleic acids in one or more AAV vectors. In some aspects, the method comprises delivering the nucleic acids via non-vectorized delivery.

In some aspects, the disclosure provides a method of delivering any one or more nucleic acids comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9 to a cell or to a subject in need thereof. In some aspects, the method comprises administering to the subject an AAV comprising one or more nucleotide sequences encoding (i) a DMD-targeted gRNA (e.g., a gRNA targeting a mutation involving, surrounding, or affecting a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7,3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), and (ii) a Cas9 enzyme. In some aspects, the nucleic acid encoding the Cas9 enzyme comprises the nucleotide sequence set forth in SEQ ID NO: 277 or 278, or a variant thereof comprising at least about 70% identity to the nucleotide sequence set forth in in SEQ ID NO: 277 or 278, or a functional fragment thereof. In some aspects, the method comprises administering to the subject a nucleic acid comprising a nucleotide sequence encoding (i) a gRNA, wherein at least one gRNA targets a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene, and (ii) a Cas9 enzyme or a functional fragment thereof. In some aspects, the method comprises delivering the nucleic acids in one or more AAV vectors. In some aspects, the method comprises delivering the nucleic acids via non-vectorized delivery.

In yet another aspect, the disclosure provides a method of increasing expression of the DMD gene or increasing the expression of a full-length dystrophin or a functional dystrophin in a cell, wherein the method comprises contacting the cell with a nucleic acid comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276 (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9 to a cell or to a subject in need thereof. In some aspects, the method comprises administering to the subject an AAV comprising one or more nucleotide sequences encoding (i) a DMD-targeted gRNA (e.g., a gRNA targeting a mutation involving, surrounding, or affecting a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7,3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), and (ii) a Cas9 enzyme. In some aspects, the nucleic acid encoding the Cas9 enzyme comprises the nucleotide sequence set forth in SEQ ID NO: 277 or 278, or a variant thereof comprising at least about 70% identity to the nucleotide sequence set forth in in SEQ ID NO: 277 or 278, or a functional fragment thereof. In some aspects, the method comprises contacting the cell with a nucleic acid comprising a nucleotide sequence encoding (i) a gRNA, wherein at least one gRNA targets a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6,3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene, and (ii) a Cas9 enzyme or a functional fragment thereof. In some aspects, the method comprises delivering the nucleic acids in one or more AAV vectors. In some aspects, the method comprises delivering the nucleic acids to the cell via non-vectorized delivery.

In yet another aspect, the disclosure provides a method of increasing expression of the DMD gene or increasing the expression of a full-length dystrophin or a functional dystrophin in a cell, wherein the method comprises contacting the cell with a nucleic acid comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9 to a cell or to a subject in need thereof. In some aspects, the method comprises administering to the subject an AAV comprising one or more nucleotide sequences encoding (i) a DMD-targeted gRNA (e.g., a gRNA targeting a duplication mutation involving, surrounding, or affecting exons 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3,3-4, 3-5, 3-6, 3-7, 3-8,3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), and (ii) a Cas9 enzyme. In some aspects, the nucleic acid encoding the Cas9 enzyme comprises the nucleotide sequence set forth in SEQ ID NO: 277 or 278, or a variant thereof comprising at least about 70% identity to the nucleotide sequence set forth in in SEQ ID NO: 277 or 278, or a functional fragment thereof. In some aspects, the method comprises contacting the cell with a nucleic acid comprising a nucleotide sequence encoding (i) a gRNA, wherein at least one gRNA targets a single or multiple exon duplication affecting exon 2 or 3 including, but not limited, to duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene), and (ii) a Cas9 enzyme or a functional fragment thereof. In some aspects, the method comprises delivering the nucleic acids in one or more AAV vectors. In some aspects, the method comprises delivering the nucleic acids via non-vectorized delivery.

In some aspects, expression of DMD or the expression of full-length dystrophin or a functional dystrophin is increased in a cell or in a subject by the methods provided herein by at least or about 5, about 10, about 15, about 20, about 25, about 30, about 35, about 40, about 45, about 50, about 55, about 60, about 65, about 70, about 75, about 80, about 85, about 90, about 95, about 96, about 97, about 98, about 99, or 100 percent.

In some aspects, the disclosure provides a recombinant gene editing complex comprising a nucleic acid comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9, which are delivered to a cell or to a subject to edit the DMD gene and correct a single exon duplication or multiple exon duplications to restore or increase full-length dystrophin expression or functional dystrophin expression in the cell or in the subject. Such gene editing complex is used for manipulating expression of DMD, increasing full-length or functional dystrophin expression, and for treating genetic disease associated with abnormal DMD expression, such as muscular dystrophy, particularly at the RNA level, where disease-relevant sequences, such as those of the DMD gene, are abhorrently expressed.

In some aspects, the disclosure provides a recombinant gene editing complex comprising a nucleic acid comprising (i) a polynucleotide encoding the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; (ii) a polynucleotide comprising the DMD gRNA comprising the nucleotide sequence set forth in any one of SEQ ID NOs: 1-184, or a variant thereof comprising at least or about 70% identity to the nucleotide sequence set forth in any one of SEQ ID NOs: 1-184; or a polynucleotide encoding a DMD gRNA targeting the nucleotide sequence set forth in any one of SEQ ID NOs: 185-276; and (iii) a nucleic acid encoding a Cas9, which are delivered to a cell or to a subject to edit the DMD gene and correct a single exon duplication or multiple exon duplications to restore or increase full-length dystrophin expression or functional dystrophin expression in the cell or in the subject. Such gene editing complex is used for manipulating expression of DMD, increasing full-length dystrophin expression or functional dystrophin expression, and for treating genetic disease associated with abnormal DMD expression, such as muscular dystrophy, particularly at the RNA level, where disease-relevant sequences, such as those of the DMD gene, are abhorrently expressed.

In some aspects, the disclosure provides AAV transducing cells for the delivery of nucleic acids comprising a nucleotide sequence encoding the gRNA and/or the Cas9 enzyme or a functional fragment thereof. Methods of transducing a target cell with AAV, in vivo or in vitro, are included in the disclosure. The methods comprise the step of administering an effective dose, or effective multiple doses, of a composition comprising an AAV of the disclosure to a subject, including an animal (such as a human being) in need thereof. If the dose is administered prior to development of the muscular dystrophy, the administration is prophylactic. If the dose is administered after the development of the muscular dystrophy, the administration is therapeutic. In embodiments of the disclosure, an effective dose is a dose that alleviates (eliminates or reduces) at least one symptom associated with the muscular dystrophy being treated, that slows or prevents progression of the muscular dystrophy, that slows or prevents progression of the muscular dystrophy, that diminishes the extent of disease, that results in remission (partial or total) of the muscular dystrophy, and/or that prolongs survival. In some aspects, the muscular dystrophy is DMD. In some aspects, the muscular dystrophy is IMD. In some aspects, the muscular dystrophy is BMD.

Combination therapies are also contemplated by the disclosure. Combination as used herein includes simultaneous treatment or sequential treatments. Combinations of methods of the disclosure with standard medical treatments (e.g., corticosteroids and/or immunosuppressive drugs) are specifically contemplated, as are combinations with other therapies such as those disclosed in International Publication No. WO 2013/016352, which is incorporated by reference herein in its entirety.

Administration of an effective dose of the compositions may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. Route(s) of administration and serotype(s) of AAV components of rAAV and scAAV (in particular, the AAV ITRs and capsid protein) of the disclosure may be chosen and/or matched by those skilled in the art taking into account the disease state being treated and the target cells/tissue(s), such as cells that express DMD. In some embodiments, the route of administration is intramuscular. In some embodiments, the route of administration is intravenous.

In particular, actual administration of AAV of the present disclosure may be accomplished by using any physical method that will transport the AAV recombinant vector into the target tissue of an animal. Administration according to the disclosure includes, but is not limited to, injection into muscle, the bloodstream, the central nervous system, and/or directly into the brain or other organ. Simply resuspending a AAV in phosphate buffered saline has been demonstrated to be sufficient to provide a vehicle useful for muscle tissue expression, and there are no known restrictions on the carriers or other components that can be co-administered with the AAV (although compositions that degrade DNA should be avoided in the normal manner with AAV). Capsid proteins of a AAV may be modified so that the AAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The AAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of AAV as a free acid (DNA contains acidic phosphate groups) or a pharmacologically acceptable salt can be prepared in water suitably mixed with a surfactant such as hydroxpropylcellulose. A dispersion of AAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. In some aspects, proper fluidity is maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

The term “transduction” is used to refer to the administration/delivery of one or more of the DMD or Cas9 constructs described herein, including, but not limited to, nucleotide sequence encoding gRNA, nucleotide sequence comprising gRNA, and one or more Cas9-encoding polynucleotides to a recipient cell either in vivo or in vitro, via a replication-deficient rAAV of the disclosure resulting in expression of the DMD gRNA and Cas9 by the recipient cell.

In one aspect, transduction with AAV is carried out in vitro. In one embodiment, desired target cells are removed from the subject, transduced with AAV and reintroduced into the subject. Alternatively, syngeneic or xenogeneic cells can be used where those cells will not generate an inappropriate immune response in the subject.

Suitable methods for the transduction and reintroduction of transduced cells into a subject are known in the art. In one embodiment, cells are transduced in vitro by combining AAV with cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

The disclosure provides methods of administering an effective dose (or doses, administered essentially simultaneously or doses given at intervals) of AAV that comprise DNA that encodes DMD gRNA targeted to restore DMD expression, and DNA that encodes Cas9 to effect cleavage of the DMD sequence to a cell or to a subject in need thereof.

Transduction of cells with AAV of the disclosure results in sustained expression of the guide RNA targeting DMD expression and the Cas9 enzyme. The disclosure thus provides methods of administering/delivering AAV which to restore full-length and/or functional dystrophin expression to a cell or to a subject. In some aspects, the cell is in a subject. In some aspects, the cell is an animal subject. In some aspects, the animal subject is a human subject.

These methods include transducing the blood and vascular system, the central nervous system, and tissues (including, but not limited to, muscle cells and neurons, tissues, such as muscle, including skeletal muscle, organs, such as heart, brain, skin, eye, and the endocrine system, and glands, such as endocrine glands and salivary glands) with one or more AAV of the present disclosure. In some aspects, transduction is carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cserjesi and Olson, Mol Cell Biol 11: 4854-4862 (1991)], control elements derived from the human skeletal actin gene [Muscat et al., Mol Cell Biol, 7: 4089-4099 (1987)], the cardiac actin gene, muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors [Semenza et al., Proc. Natl. Acad. Sci. USA, 88: 5680-5684 (1991)], steroid-inducible elements and promoters including the glucocorticoid response element (GRE) [See Mader and White, Proc. Natl. Acad. Sci. USA, 90: 5603-5607 (1993)], the tMCK promoter [see Wang et al., Gene Therapy, 15: 1489-1499 (2008)], the CK6 promoter [see Wang et al., supra] and other control elements.

Because AAV targets every affected organ expressing DMD, the disclosure includes the delivery of DNAs as described herein to all cells, tissues, and organs of a subject. In some aspects, muscle tissue, including skeleton-muscle tissue, is an attractive target for in vivo DNA delivery. The disclosure includes the sustained expression of the DMD gene to express dystrophin from transduced cells. In some aspects, the disclosure includes sustained expression of dystrophin from transduced myofibers. By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells, in some aspects, are differentiated or undifferentiated, such as myoblasts, myocytes, myotubes, cardiomyocytes and cardiomyoblasts.

In some aspects, a method of treating muscular dystrophy in a subject or patient is provided. In some aspects, “treating” includes ameliorating, inhibiting, or even preventing one or more symptoms of a muscular dystrophy, including a Duchenne muscular dystrophy, (including, but not limited to, muscle wasting, muscle weakness, myotonia, skeletal muscle problems, heart function abnormalities, breathing difficulties, issues with speech and swallowing (dysarthria and dysphagia) or cognitive impairment), abnormalities of the retina, hip weakness, facial weakness, abdominal muscle weakness, joint and spinal abnormalities, lower leg weakness, shoulder weakness, hearing loss, muscle inflammation, and nonsymmetrical weakness.

In some aspects, a method of treating results in increased expression of dystrophin protein or increased expression of an altered form or fragment of dystrophin protein that is physiologically or functionally active in the subject. In some aspects, the dystrophin is a full-length dystrophin, or a functional form of dystrophin which prevents, ameliorates, or treats a muscular dystrophy which would result or results from the mutation in the DMD gene. In some aspects, the dystrophin is a shorter, usable dystrophin which, in some aspects, makes the effects of such DMD mutation less severe. In particular aspects, the method of treating inhibits the progression of dystrophic pathology in the subject. In some aspects, the method of treating improves muscle function in the subject. In some aspects, the improvement in muscle function is an improvement in muscle strength. In some aspects, the improvement in muscle function is an improvement in stability in standing and walking. The improvement in muscle strength is determined by techniques known in the art, such as the maximal voluntary isometric contraction testing (MVICT). In some instances, the improvement in muscle function is an improvement in stability in standing and walking. In some aspects, an improvement in stability or strength is determined by techniques known in the art such as the 6-minute walk test (6MWT), the 100 meter run/walk test, or timed stair climb.

Molecular, biochemical, histological, and functional endpoints demonstrate the therapeutic efficacy of the products and methods disclosed herein for increasing the expression of the DMD gene. Endpoints contemplated by the disclosure include increasing DMD (dystrophin) protein expression, which has application in the treatment of muscular dystrophies including, but not limited to, DMD, IMD, and BMD and other disorders associated with absent or reduced DMD expression.

The disclosure also provides kits for use in the treatment of a disorder described herein. Such kits include at least a first sterile composition comprising any of the nucleic acids described herein above or any of the viral vectors described herein above in a pharmaceutically acceptable carrier. Another component is optionally a second therapeutic agent for the treatment of the disorder along with suitable container and vehicles for administrations of the therapeutic compositions. The kits optionally comprise solutions or buffers for suspending, diluting or effecting the delivery of the first and second compositions.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

All methods described herein are performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

Design and Generation of gRNA Sequences that Target DMD

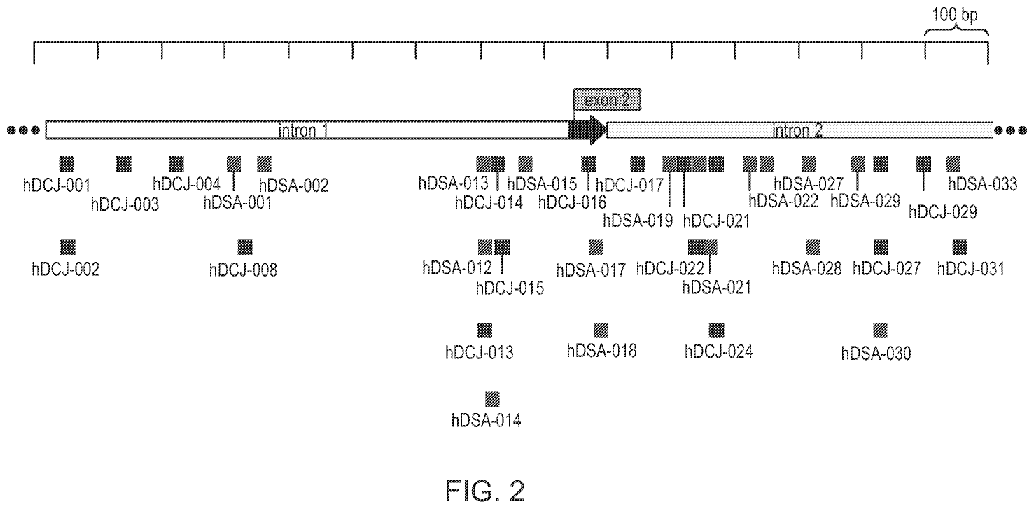

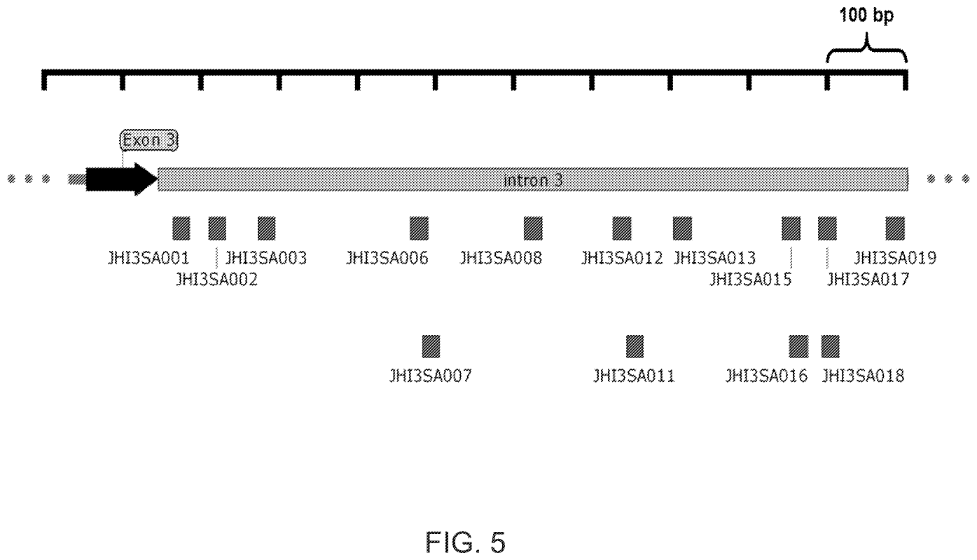

In order to test the ability to correct DMD exon duplications, various gRNA sequences were designed to be used in conjunction with CRISPR-Cas9 to target the DMD gene (see FIG. 2 and FIG. 5). Table 1 provides Staphylococcus aureus and Campylobacter jejuni gRNA nucleotide sequences designed to target human and mouse DMD exons and the flanking intronic sequences of the DMD gene.

Sequences in Table 1 were designed by searching the most current human (GRCh38.p12) and mouse (GRCm38.p6) genomic DNA reference builds for SaCas9 (5′-NNGRRT-3′) (SEQ ID NO: 279) and CjCas9 (5′-NNNNRYAC-3′) (SEQ ID NO: 280) PAM sequences within a 2000 base pair window spanning exon 2 (in intron 1, intron 2, and exon 2) and a 1000 bp window downstream of exon 3 (in intron 3). Next, potential gRNA spacer sequences corresponding to the PAM sites were gathered by collecting 22 bases upstream of the PAM sequences on the same strand and then converting them to RNA sequences. For those that did not begin with a 5′ guanine residue, an additional 5′ guanine was added to drive efficient RNA polymerase Ill transcription initiation. A three-step in silico exclusion pipeline was applied to reduce the library size while selecting for gRNAs with the highest potential gene editing efficiency and site specificity. First, the spacer sequences were screened for RNA polymerase Ill termination signals (5′-UUUUU-3′) (SEQ ID NO: 281) and excluded from further testing if they contained one or more homopolymeric sequences of five or more uracil residues. The RNA polymerase Ill-based constraints were required to drive efficient gRNA transcription from the U6 small nuclear RNA promoter used for expression. Second, gRNAs were excluded from further analysis if they targeted a genomic DNA region containing one or more common (>1% minor allele frequency) single nucleotide polymorphisms that could hinder gRNA activity for patients carrying the minor allele. Last, gRNAs were excluded if they had greater than 30 predicted off-target sites, or one or more off-target sites within an exon as predicted using the CCTop online webtool and searching within the appropriate genomic DNA reference build (either human or mouse).

### Example 2

### Experimental Materials and Methods

### Molecular Cloning

The SaCas9 and CjCas9 gRNA sequences were synthesized by Twist Bioscience as double stranded DNA fragments within U6 promoter-driven expression cassettes flanked by BamHI and XbaI restriction enzyme sites. A custom plasmid containing cytomegalovirus promoter-driven SaCas9 sequence and a U6-promoter driven SaCas9 gRNA expression cassette flanked by BamHI and XhoI sites was produced by Vector Builder Inc. The CjCas9 sequence was synthesized as three fragments by Twist Bioscience and assembled between the SnaBI and PsiI sites in place of SaCas9 in the Vector Builder Inc custom plasmid using In-Fusion Cloning (Takara Bio Inc). Each SaCas9 or CjCas9 gRNA was sub-cloned into the corresponding SaCas9 or CjCas9 plasmid using Roche rAPid DNA Dephos & Ligation Kit with the BamHI and XhoI sites. All plasmids were confirmed via Sanger sequencing.

For AAV plasmid cloning, a plasmid encoding AAV serotype 2 ITRs, a multiple cloning site, and a human growth hormone polyadenylation signal (hGHpA) was purchased from Cell Bio Labs (pAAV-MCS). To prepare the MHCK7 promoter-driven SaCas9 expression cassette, PCR was used to remove the CMV promoter and linearize the SaCas9 Vector Builder Inc custom plasmid. The MHCK7 promoter sequence was then sub-cloned in placed of the CMV promoter using In-Fusion Cloning (Takara Bio Inc). PCR was then used to amplify the MHCK7-promoter and SaCas9 coding sequence from the plasmid as well as add EcoRI and XbaI sites on the ends. The amplicon was then sub-cloned into the pAAV-MCS plasmid between the ITRs, upstream of the hGHpA using Roche rAPid DNA Dephos & Ligation Kit with EcoRI and XbaI restriction sites. To generate the scAAV containing three U6-driven gRNA expression cassettes, PCR was used to amplify a U6-driven gRNA expression cassette and add NheI and NotI restriction sites onto the ends. After subcloning the single cassette between the NheI and NotI restriction sites within a self-complimentary AAV backbone using Roche rAPid DNA Dephos & Ligation Kit, additional PCRs were used to generate U6-mDS010 amplicons with NheI sites on both ends and NotI sites on both ends. These amplicons were then individually sub-cloned at their respective restriction sites with Roche rAPid DNA Dephos & Ligation Kit to generate the three copy U6-gRNA scAAV plasmids.

In some aspects, all-in-one AAV plasmids are constructed. For the all-in-one AAV plasmids containing CMV-driven Sa Cas9, a custom plasmid encoding CMV-driven Sa Cas9 and U6-driven hDSA-018 gRNA flanked by NotI restriction sites was generated by GenScript. The plasmid was digested with NotI and the CMV-SaCas9-U6-hDSA-018 fragment sub-cloned between the ITRs into the pAAV-MCS plasmid to generate the plasmid for producing pAAV-AIO-hDSA018. BamHI and XhoI sites flanking the U6-hDSA-018 sequence were used to replace the U6-hDSA-018 with U6-mDSA-004 and U6-hDSA-017 to generate plasmids for production of pAAV-AIO-mDSA004 and pAAV-AIO-hDSA017, respectively.

All plasmids sequences were confirmed via restriction fragment lengths and Sanger sequencing.

### Cell Culture and Treatments

HEK293 cells were cultured in plastic 10 cm petri dishes with Corning DMEM with L-glutamine, 4.5 g/L glucose and sodium pyruvate supplemented with 10% HyClone Cosmic Calf Serum, 1% Gibco MEM Non-Essential Amino Acids Solution (100×), and 1% Gibco Antibiotic-Antimycotic (100×). Cells were routinely passed upon reaching 80% confluency using Gibco 0.05% trypsin-EDTA solution. For transfections, Invitrogen Lipofectamine LTX with Plus Reagent was used according to the manufacturer's suggested protocol for HEK293 cells. Patient skin fibroblast cells immortalized with human telomerase reverse transcriptase and modified with a doxycycline-inducible myoblast determination protein 1 using lentiviruses (FibroMyoD cells) were cultured in DMEM with L-glutamine, 4.5 g/L glucose and sodium pyruvate supplemented with 20% HyClone Fetal Bovine Serum, and 1% Gibco Antibiotic-Antimycotic (100×). Cells were routinely passed upon reaching 80% confluency using Gibco 0.05% trypsin-EDTA solution. For transdifferentiation into myotubes, culture medium was switched upon FibroMyoD cells reaching 60% confluence to PromoCell Skeletal Muscle Cell Growth Medium supplemented with 8 μg/mL doxycycline for three days. Medium was then switched to Skeletal Muscle Cell Differentiation Medium (PromoCell) supplemented with 8 μg/mL doxycycline for 14 days.

In Vitro Screening of gRNAs

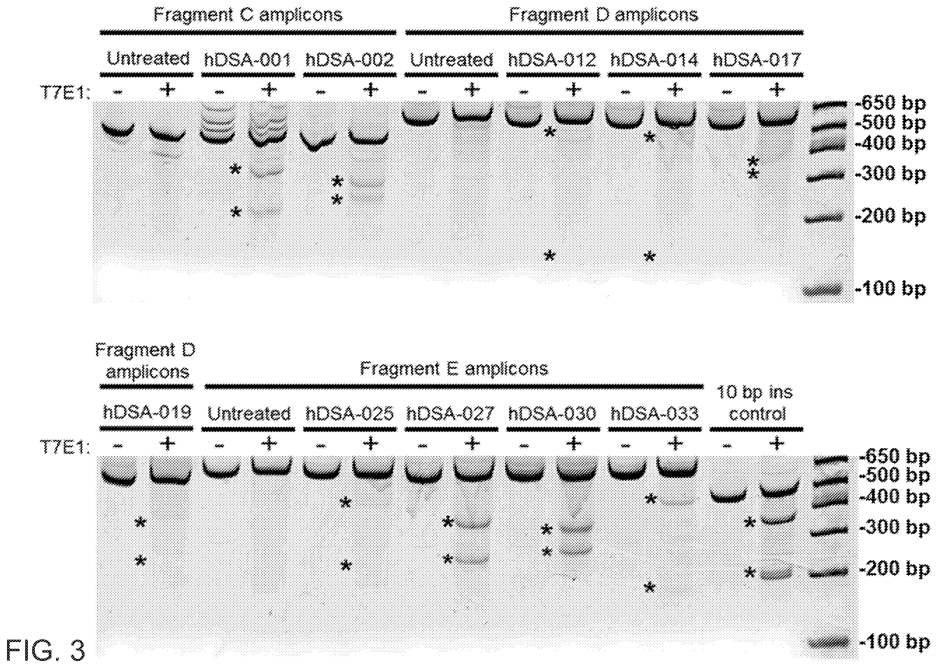

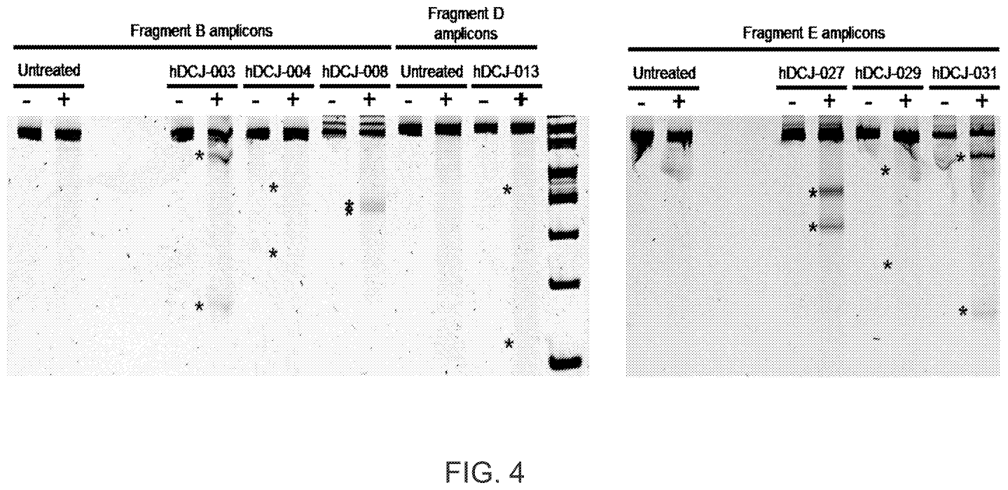

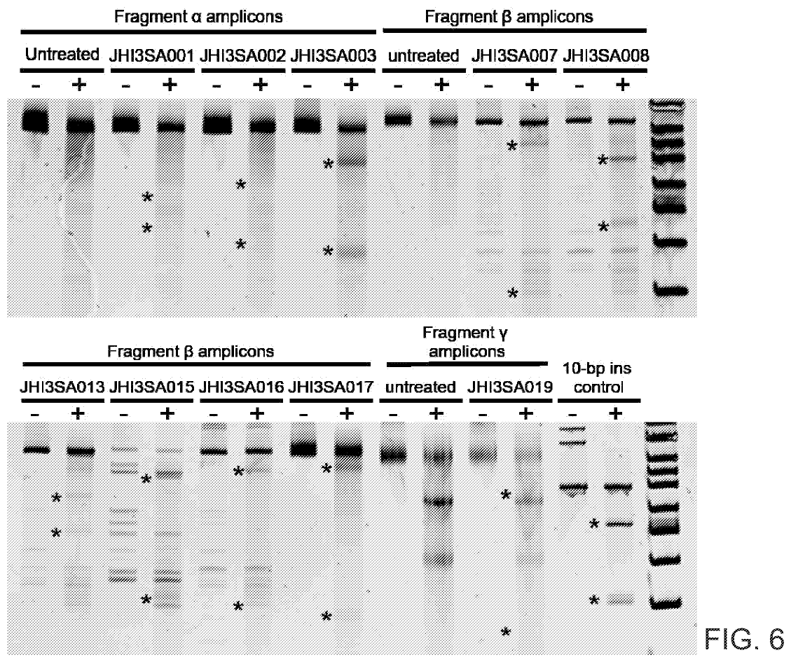

HEK293 cells were plated in 12-well plastic tissue culture dishes (200,000 cells/well) and cultured overnight. Cells were transfected with a plasmid encoding cytomegalovirus promoter-driven Sa or CjCas9 and a U6-promoter driven SaCas9 gRNA or CjCas9 gRNA expression cassette using Lipofectamine LTX with Plus Reagent (Invitrogen) according to the manufacturer's suggested protocol for HEK293 cells. After six hours, the culture medium was replaced and the cells were cultured an additional 72 hours. Cells were collected using 0.05% trypsin-EDTA solution (Gibco) and genomic DNA was extracted using the DNeasy Blood & Tissue Kit (Qiagen). PCR was used to generate amplicons spanning the CRISPR-Cas9 target locations which were used with EnGen Mutation Detection Kit (NEB) to detect CRISPR-Cas9 induced insertions and deletions. Cleavage products were resolved with electrophoresis using a 10% polyacrylamide tris-borate-EDTA gel, stained with a solution of 0.5 μg/mL ethidium bromide, and imaged with UV transillumination on a ChemiDoc Imaging System (Bio-Rad). Gene editing was detectable as cleavage of the amplicons at predicted sites of mutations (see FIG. 3, FIG. 4, and FIG. 6). Those gRNAs that resulted in detectable gene editing were deemed hits.

### Immunofluorescence Staining

Mouse tibialis anterior muscles were dissected and mounted in tragacanth gum before snap freezing in liquid nitrogen-cooled isopentane. Cross-sections (10 microns) were collected using a microtome at −20° C. Sections were permeablized in phosphate buffered saline (PBS) supplemented with 10% normal goat serum and 0.1% Triton X-100 for 10 minutes before washing with three volumes of PBS for 5 minutes each. Sections were blocked with PBS supplemented with 10% normal goat serum and 0.1% Tween 2. Sections were stained with a 1:400 dilution of rabbit anti-dystrophin antibody (AB15277; Abcam) and 1:400 dilution of rat anti-laminin antibody (MAB4656; R&D Systems) in phosphate buffered saline supplemented with 10% normal goat serum and 0.1% Tween 2 for 2 hours. After washing with four volumes of 0.1% Tween-20 in PBS for 5 minutes each, sections were stained with a 1:500 dilution of Alexa 568-labeled donkey anti-rat antibody (712-546-153) and 1:500 dilution of Alexa 488-labeled goat anti-rabbit antibody (A-21069) in phosphate buffered saline supplemented with 10% normal goat serum and 0.1% Tween 2 for 1 hour. Sections were then washed with three volumes of 0.1% Tween-20 in PBS for 5 minutes each. Sections were mounted in ProLong Gold Antifade Mountant with DAPI.

### Immunofluorescence Imaging and Analysis

Whole muscle cross-section images were collected with a Nikon Ti2E fluorescence microscope in blue (DAPI), green (Alexa 488; Thermo Fisher Scientific), and red (Alexa 568; Thermo Fisher Scientific) fluorescence channels through a 10× objective. Analysis was carried out in Nikon NIS-Elements AR software using the General Analysis 3 software module and a custom analysis workflow developed for mouse tissue. Skeletal muscles were analyzed as whole tissue sections, using thresholds for dystrophin-positive and laminin-positive pixels that were empirically derived from the intensity profiles of both signals in untreated dup2 (mouse model of exon 2 duplication) mouse tissue sections. Dystrophin-positive fibers were quantified by identifying all individual muscle fibers using laminin-positive boundaries, measuring the total length of dystrophin-positive segments around each muscle fiber, and normalizing it to the total length of the laminin-positive segment around the muscle fiber perimeter. The criterion for identifying a muscle fiber as overall positive for dystrophin was set at 70% or more of the perimeter.

In Vivo Screening of gRNAs

Mouse-targeting gRNAs are screened for activity in vivo by intramuscular injection of AAV1 encoding CMV or MHCK7 promoter-driven Sa or Cj Cas9 and U6 promoter-driven gRNA into the TA muscles of a mouse model of exon 2 duplication (dup2 mice). After 4 weeks, the TAs are collected, mounted, stained, and imaged as described herein above. The active mouse-targeting gRNAs result in expression of dystrophin (>2% dystrophin-positive fibers) in injected muscles while inactive gRNAs result in no dystrophin expression (<2% positive fibers). The level of dystrophin expression is directly proportional to gene editing activity of the individual gRNAs.

### Example 3

### Correcting DMD Exon 2 Duplications and Multiexon Duplications in Patient Cells

The objective of these experiments was to test whether an AAV vectorized Cas9 and gRNA delivery system could induce collapse of exon 2 and multiexon duplications in patient derived cells. To this end, a recombinant AAV encoding a muscle-specific expression cassette for SaCas9 driven by a synthetic promoter comprised of the myosin heavy chain enhancer and creatine kinase core promoter (MHCK7 promoter, doi: 10.1038/sj.mt.6300027) and human growth hormone polyadenylation signal was constructed. A second AAV was constructed and used to encode three copies of human U6-promoter driven hDSA030 gRNA expression cassettes. The gRNA AAV is a self-complimentary AAV genome in that it carries a mutated inverted terminal repeat (ITR) lacking a terminal resolution site which results in packaging of a double-stranded genome instead of a single-stranded genome typical of AAV which has been shown to enhance CRISPR-Cas9 gene editing in vivo (doi: 10.1038/sj.gt.3302134). FibroMyoD cells from a patient with an exon 2 duplication (Dup2) and a patient with an exon 2 through 6 duplication (Dup2-6) were treated as indicated in Table 3 (n=3 biological replicates) using mixtures of the two AAV viruses (high (H), medium (M), and low (L) dose groups indicated in Table 3) or mock treated without AAV virus (untreated).

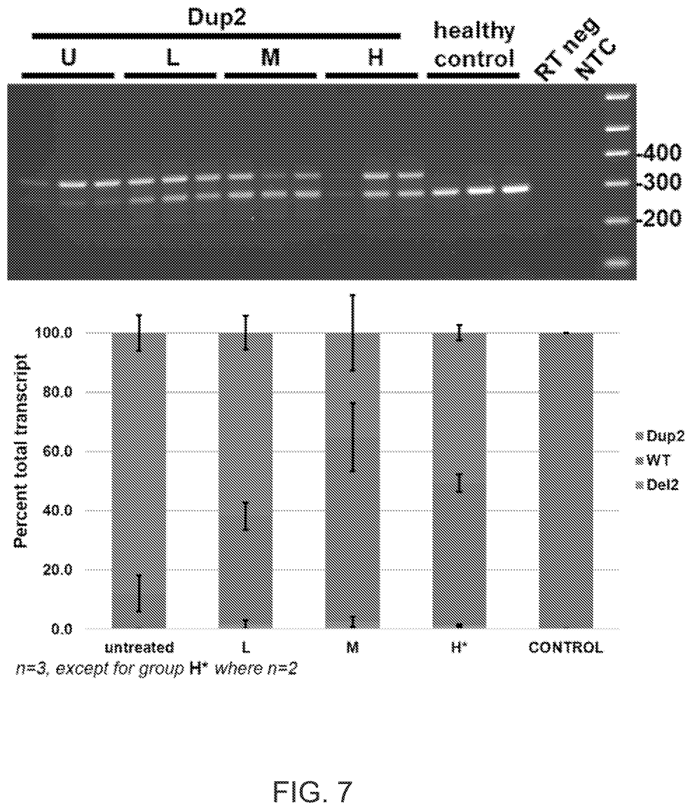

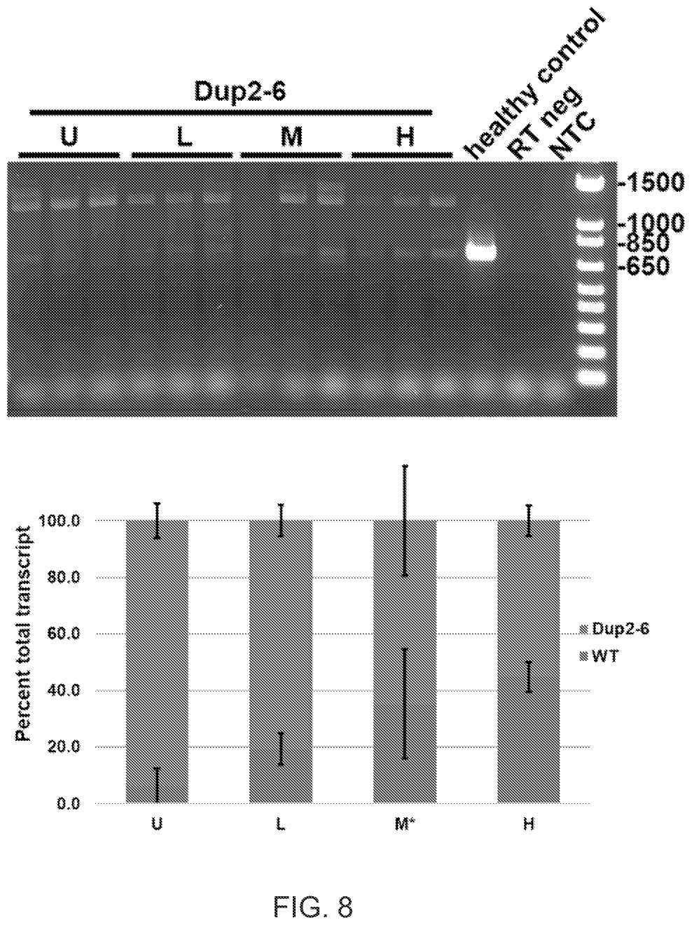

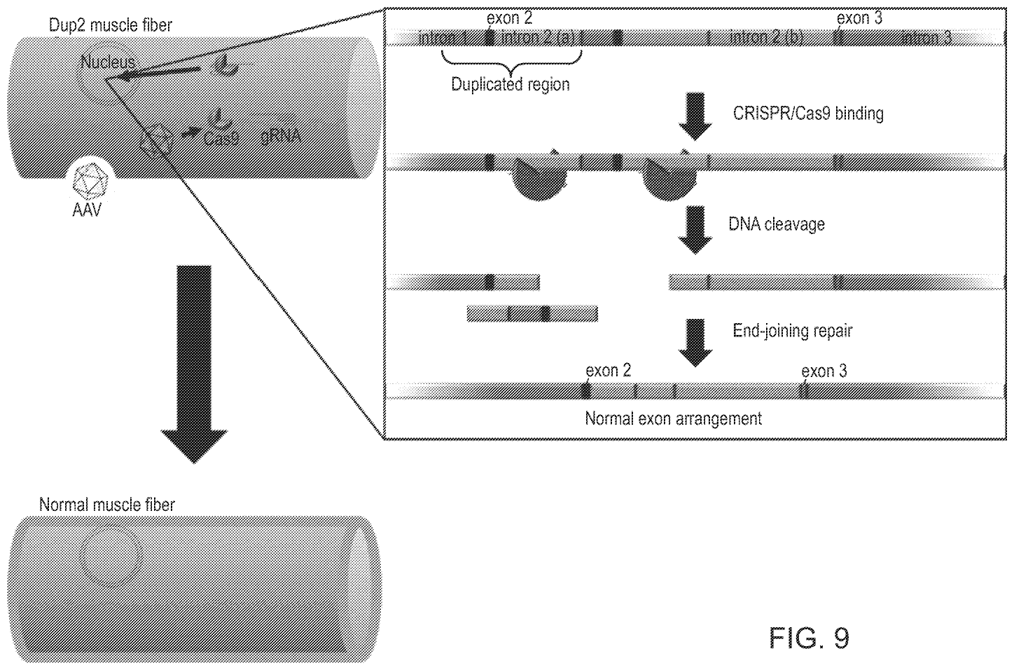

Briefly, the cells were plated and allowed to reach ˜60% confluency before switching the medium to Muscle Cell Growth Medium (PromoCell) supplemented with 8 μg/mL doxycycline. After 3 days, the medium was replaced with Muscle Cell Differentiation Medium (PromoCell) supplemented with 8 μg/mL doxycycline, Cas9 AAV (rAAV1.MHCK7.SaCas9.hGHpA), and gRNA AAV (scAAV1.3xU6.hDSA030) as indicated in Table 3. After 72 hours (and every 2-3 days thereafter), the medium was replaced with fresh Muscle Cell Differentiation Medium (PromoCell) supplemented with 8 μg/mL doxycycline. The cells were transdifferentiated for two weeks before collection of whole RNA using TRIzol reagent (Invitrogen) and RNA Clean & Concentrator™-25 Kit (Zymo Research). The RNA (1 μg) was used to prepare cDNA with RevertAid First Strand cDNA Synthesis Kit (Thermo Scientific) in 20 μL reactions. PCR was performed using a forward primer that anneals to the DMD 5′ untranslated region and a reverse primer that anneals to exon 3 (for Dup2 cells) or exon 8 (for Dup2-6 cells) (FIG. 7 and FIG. 8). Importantly, Dup2 the FibroMyoD cell line from the Dup2 patient exhibited a significant amount of natural exon 2 skipping (˜15% of DMD transcripts).

TABLE 3
Treatment of patient cells with AAV-vectorized CRISPR-Cas9.

| | | | Titer during |
|---|---|---|---|
| | | MOI | treatment |
| Group ID | AAV Name | (vg/cell) | (vg/mL) |
| H | rAAV1.MHCK7.SaCas9.hGHpA | 1.50E+06 | 8.6E+11 |
| | scAAV1.3xU6.hDSA030 | 1.50E+06 | 8.6E+11 |
| M | rAAV1.MHCK7.SaCas9.hGHpA | 5.00E+05 | 2.9E+11 |
| | scAAV1.3xU6.hDSA030 | 5.00E+05 | 2.9E+11 |
| L | rAAV1.MHCK7.SaCas9.hGHpA | 1.00E+05 | 5.7E+10 |
| | scAAV1.3xU6.hDSA030 | 1.00E+05 | 5.7E+10 |
| Untreated | rAAV1.MHCK7.SaCas9.hGHpA | 0.00E+00 | 0.0E+00 |
| | scAAV1.3xU6.hDSA030 | 0.00E+00 | 0.0E+00 |

Treatment of Dup2 cells with the low dose at a multiplicity of infection (MOI) of 2.0×10⁵ total AAV per cell (1:1 Cas9:gRNA AAV ratio) induced an approximately 2.5-fold increase to ˜38% of wild-type transcripts resulting from deletion of the extra copy of exon 2 and the intervening intronic sequences (>150 kb). A dose response was observed with increased MOI to the medium dose of 1×10⁶ total AAV per cell resulting in >65% of transcripts corresponding to the wild-type exon arrangement. However, a further increase to the high MOI dose of 3×10⁶ total AAV per cell did not result in increased editing, potentially due to saturation of AAV binding, expression of Cas9 and/or gRNA, or DMD target sites. In Dup2-6 cells, a similar trend was observed for deletion of the >210 kb Dup2-6 locus with an increase in wild-type exon arrangement from <10% in untreated cells to ˜50% at the highest AAV dose. Potential saturation of editing was also observed with an insignificant improvement between medium and high doses.

### Example 4

Intramuscular Delivery of rAAV and scAAV Comprising Nucleotide Sequences Encoding SaCas9 and mDSA010 gRNA Results in Dose-Dependent Increased Expression of Full-Length Dystrophin

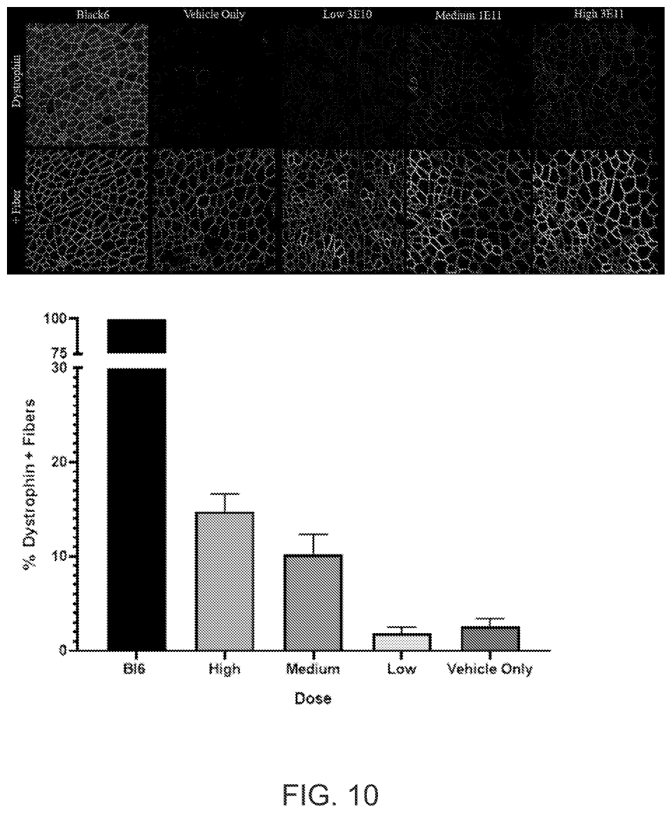

To test whether the designed AAV-vectorized Cas9 and gRNA delivery system could induce correction of the exon 2 duplication mutation in a Dup2 mouse model, both tibialis anterior (TA) muscles of 4-week-old Dup2 mice were injected with various doses (high (H), medium (M), and low (L) doses, as indicated in Table 4, of 1:1 mixtures of two AAVs, as indicated in Table 4 (n=4 biological replicates). The AAV mixture comprised a recombinant AAV serotype 9 encoding MHCK7 promoter-driven SaCas9 (rAAV9.MHCK7.SaCas9.hGHpA) and a self-complimentary AAV serotype 9 encoding three copies of U6 promoter-driven mDSA010 gRNAs (scAAV9.3xU6.mDSA010). As untreated controls, dup2 mice were also injected with the buffer formulation (group V). After 4 weeks, the injected TAs were collected and dystrophin expression was analyzed by immunofluorescence microscopy following imaging of whole muscle cross sections co-stained with antibodies against dystrophin in the red channel (FIG. 10) and laminin in the green channel (not shown) (FIG. 10).

TABLE 4
Injection of dup2 mouse tibialis anterior
muscles with AAV-vectorized CRISPR-Cas9.

| Group | | Final dose per muscle |
|---|---|---|
| ID | AAV Name | (vg) |
| H | rAAV9.MHCK7.SaCas9.hGHpA | 3.0E+11 |
| | scAAV9.3xU6.mDSA010 | 3.0E+11 |
| M | rAAV9.MHCK7.SaCas9.hGHpA | 1.0E+11 |
| | scAAV9.3xU6.mDSA010 | 1.0E+11 |
| L | rAAV9.MHCK7.SaCas9.hGHpA | 3.0E+10 |
| | scAAV9.3xU6.mDSA010 | 3.0E+10 |
| V | None | 0.0E+00 |
| | None | 0.0E+00 |

First, a threshold value was determined using a custom analysis script in Nikon Elements AR software for the red (dystrophin) and green (laminin) channels using the 99th and the 66th percentile pixel intensity values, respectively. These threshold values were averaged from all IF images of vehicle-injected dup2 muscles and then used in a separate custom analysis script to measure dystrophin-positive fibers. Briefly, as it localizes to the sarcolemma-like dystrophin, laminin was used to mark the sarcolemma of individual muscle fibers on whole muscle section images with a coordinate mask. The fiber sarcolemma coordinate mask was then used to measure properly-localized dystrophin for all individual muscle fibers in each image. Next, muscle fibers with at least 30% of their sarcolemma perimeter containing red channel pixel intensity above the red channel threshold value were counted as dystrophin positive. It was found that buffer-injected or low dose (6×10¹⁰ total AAV per muscle) injected mouse TAs contained only ˜2% dystrophin positive fibers while injection of the medium dose (2×10¹¹ total AAV per muscle) or high dose (6×10¹¹ total AAV per muscle) of the 1:1 CRISPR-Cas9 AAV mixture resulted in ˜10% and ˜15% dystrophin-positive fibers, respectively (FIG. 10).

In conclusion, delivery of the AAV vectorized CRISPR-Cas9 sequences was able to induce large deletions (˜30 kb) to correct the dup2 mutation in living dystrophic mouse skeletal muscle tissue and restore significant dystrophin expression.

### Example 5

In Vivo Screening of gRNAs

The gRNAs of Table 1 are screened for activity in vivo by intramuscular injection of AAV1 encoding MHCK7 promoter-driven Sa or Cj Cas9 and U6 promoter-driven gRNA into the TA muscles of a mouse model of exon 2 duplication (dup2 mice). After 4 weeks, the TAs are collected, mounted, stained, and imaged as described herein above. The active DMD-targeting gRNAs of Table 1 result in expression of dystrophin (>2% dystrophin-positive fibers) in injected muscles while inactive gRNAs result in no dystrophin expression (<2% positive fibers). The level of dystrophin expression is directly proportional to gene editing activity of the individual gRNAs.

### Example 6

### Correcting Exon 2 Duplications and Exon 2-6 Multiexon Duplication in Patient-Derived Cells

The objective of these experiments was to further test whether AAV vectorized Cas9 and gRNAs could induce removal of a duplicate copy of exon 2 and multiexon duplication of exons 2-6 in patient derived cells. To this end, a recombinant AAV encoding a muscle-specific expression cassette for SaCas9 driven by a synthetic promoter comprised of the myosin heavy chain enhancer and creatine kinase core promoter (MHCK7 promoter, doi: 10.1038/sj.mt.6300027) and human growth hormone polyadenylation signal was constructed. A second AAV was constructed and used to encode three copies of human U6-promoter driven hDSA-030 gRNA (SEQ ID NO: 30) expression cassettes. The gRNA AAV is a self-complimentary AAV genome in that it carries a mutated inverted terminal repeat (ITR) lacking a terminal resolution site which results in packaging of a double-stranded genome instead of a single-stranded genome typical of AAV which has been shown to enhance CRISPR-Cas9 gene editing in vivo (doi: 10.1038/sj.gt.3302134). FibroMyoD cells from two patients with exon 2 duplication (Dup2) and one patient with an exon 2 through 6 duplication (Dup2-6) were treated using a mixture of the two AAV viruses in a 1:1 ratio at a total MOI of 4E6 vg/cell (treated) or mock treated without AAV virus (untreated).

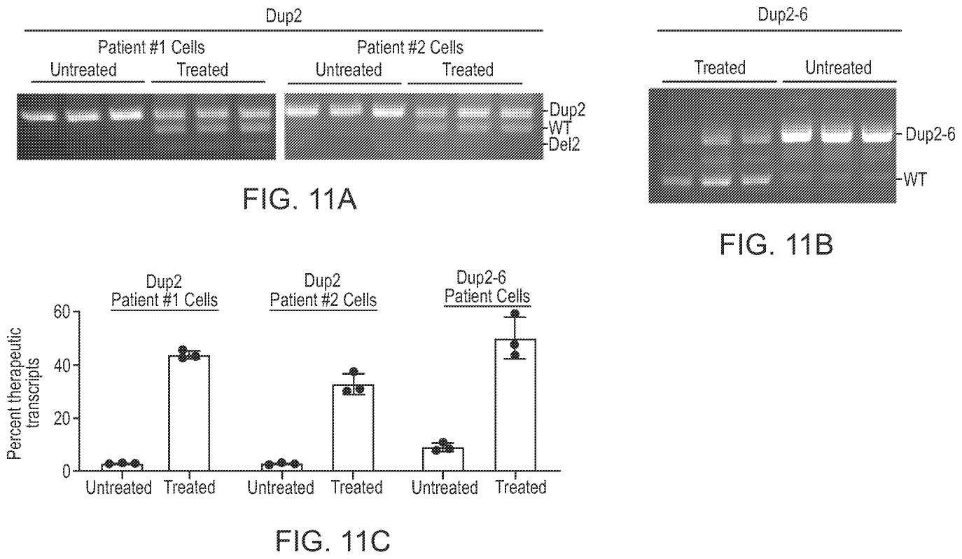

Briefly, the cells were plated and allowed to reach ˜60% confluency before switching the medium to Muscle Cell Growth Medium (PromoCell) supplemented with 8 μg/mL doxycycline. After 3 days, the medium was replaced with Muscle Cell Differentiation Medium (PromoCell) supplemented with 8 μg/mL doxycycline containing the Cas9 AAV (rAAV1.MHCK7.SaCas9.hGHpA), and gRNA AAV (scAAV1.3xU6.hDSA030). After 72 hours (and every 2-3 days thereafter), the medium was replaced with fresh Muscle Cell Differentiation Medium (PromoCell) supplemented with 8 μg/mL doxycycline. The cells were transdifferentiated for two weeks before collection of whole RNA using TRIzol reagent (Invitrogen) and RNA Clean & Concentrator™-25 Kit (Zymo Research). The RNA (1 μg) was used to prepare cDNA with RevertAid First Strand cDNA Synthesis Kit (Thermo Scientific) in 20 μL reactions. PCR was performed using a forward primer that anneals to the DMD 5′ untranslated region and a reverse primer that anneals to exon 3 (for Dup2 cells) or exon 8 (for Dup2-6 cells) (FIG. 11A-C).

Treatment of Dup2 cells with the technology described and a gRNA comprising hDSA030 (SEQ ID NO: 30), resulted in between 31-46% of DMD transcripts corresponding to a therapeutic exon arrangement without the extra copy of exon 2. Importantly, deletion of exon 2 (del2) was detected in Patient #1 cells after treatment (˜10% of total transcripts) and is considered a therapeutic transcript due to an IRES-driven dystrophin protein isoform that has shown to confer normal function by the serendipitous discovery of del2 in several individuals without muscular dystrophy. As hDSA-030 gRNA targets DMD intron 2, del2 may occur when additional bases are deleted around the gRNA target site during DNA repair in a subset of cells. In Dup2-6 cells, a similar trend was observed where deletion of the >210 kb Dup2-6 locus with an increase in wild-type exon arrangement from <10% in untreated cells to >50% after treatment.

While the present disclosure has been described in terms of specific embodiments, it is understood that variations and modifications will occur to those skilled in the art. Accordingly, only such limitations as appear in the claims should be placed on the disclosure.

All documents or references referred to in this application are hereby incorporated by reference in their entirety with particular attention to the content for which they are referred.

### REFERENCES

C. H. Hakim, N. B. Wasala, C. E. Nelson, L. P. Wasala, Y. Yue, J. A. Louderman, T. B. Lessa, A. Dai, K. Zhang, G. J. Jenkins, M. E. Nance, X. Pan, K. Kodippili, N. N. Yang, S. J. Chen, C. A. Gersbach, D. Duan, AAV CRISPR editing rescues cardiac and muscle function for 18 months in dystrophic mice. JCI insight 3, (2018); published online Epub December 6 (10.1172/jci.insight.124297).
L. Xu, Y. Gao, Y. S. Lau, R. Han, Adeno-Associated Virus-Mediated Delivery of CRISPR for Cardiac Gene Editing in Mice. Journal of visualized experiments: JoVE, (2018); published online Epub August 2 (10.3791/57560).
I. Maggio, J. Liu, J. M. Janssen, X. Chen, M. A. Goncalves, Adenoviral vectors encoding CRISPR-Cas9 multiplexes rescue dystrophin synthesis in unselected populations of DMD muscle cells. Scientific reports 6, 37051 (2016); published online Epub November 15 (10.1038/srep37051).
C. Long, H. Li, M. Tiburcy, C. Rodriguez-Caycedo, V. Kyrychenko, H. Zhou, Y. Zhang, Y. L. Min, J. M. Shelton, P. P. A. Mammen, N. Y. Liaw, W. H. Zimmermann, R. Bassel-Duby, J. W. Schneider, E. N. Olson, Correction of diverse muscular dystrophy mutations in human engineered heart muscle by single-site genome editing. Science advances 4, eaap9004 (2018); published online Epub January (10.1126/sciadv.aap9004).
A. Lattanzi, S. Duguez, A. Moiani, A. Izmiryan, E. Barbon, S. Martin, K. Mamchaoui, V. Mouly, F. Bernardi, F. Mavilio, M. Bovolenta, Correction of the Exon 2 Duplication in DMD Myoblasts by a Single CRISPR-Cas9 System. Molecular therapy. Nucleic acids 7, 11-19 (2017); published online Epub June 16 (10.1016/j.omtn.2017.02.004).
P. Zhu, F. Wu, J. Mosenson, H. Zhang, T. C. He, W. S. Wu, CRISPR-Cas9-Mediated Genome Editing Corrects Dystrophin Mutation in Skeletal Muscle Stem Cells in a Mouse Model of Muscle Dystrophy. Molecular therapy. Nucleic acids 7, 31-41 (2017); published online Epub June 16 (10.1016/j.omtn.2017.02.007).
H. Mou, J. L. Smith, L. Peng, H. Yin, J. Moore, X. O. Zhang, C. Q. Song, A. Sheel, Q. Wu, D. M. Ozata, Y. Li, D. G. Anderson, C. P. Emerson, E. J. Sontheimer, M. J. Moore, Z. Weng, W. Xue, CRISPR-Cas9-mediated genome editing induces exon skipping by alternative splicing or exon deletion. Genome biology 18, 108 (2017); published online Epub June 14 (10.1186/s13059-017-1237-8).
Y. Zhang, C. Long, H. Li, J. R. McAnally, K. K. Baskin, J. M. Shelton, R. Bassel-Duby, E. N. Olson, CRISPR-Cpf1 correction of muscular dystrophy mutations in human cardiomyocytes and mice. Science advances 3, e1602814 (2017); published online Epub April (10.1126/sciadv.1602814).
B. L. Duchene, K. Cherif, J. P. Iyombe-Engembe, A. Guyon, J. Rousseau, D. L. Ouellet, X. Barbeau, P. Lague, J. P. Tremblay, CRISPR-Induced Deletion with SaCas9 Restores Dystrophin Expression in Dystrophic Models In Vitro and In Vivo. Molecular therapy: the journal of the American Society of Gene Therapy 26, 2604-2616 (2018); published online Epub November 7 (10.1016/j.ymthe.2018.08.010).
L. Xu, K. H. Park, L. Zhao, J. Xu, M. El Refaey, Y. Gao, H. Zhu, J. Ma, R. Han, CRISPR-mediated Genome Editing Restores Dystrophin Expression and Function in mdx Mice. Molecular therapy: the journal of the American Society of Gene Therapy 24, 564-569 (2016); published online Epub March (10.1038/mt.2015.192).
D. J. Truong, K. Kuhner, R. Kuhn, S. Werfel, S. Engelhardt, W. Wurst, O. Ortiz, Development of an intein-mediated split-Cas9 system for gene therapy. Nucleic acids research 43, 6450-6458 (2015); published online Epub July 27 (10.1093/nar/gkv601).
Y. Yang, L. Wang, P. Bell, D. McMenamin, Z. He, J. White, H. Yu, C. Xu, H. Morizono, K. Musunuru, M. L. Batshaw, J. M. Wilson, A dual AAV system enables the Cas9-mediated correction of a metabolic liver disease in newborn mice. Nature biotechnology 34, 334-338 (2016); published online Epub March (10.1038/nbt.3469).
M. Hagan, M. Ashraf, I. M. Kim, N. L. Weintraub, Y. Tang, Effective regeneration of dystrophic muscle using autologous iPSC-derived progenitors with CRISPR-Cas9 mediated precise correction. Medical hypotheses 110, 97-100 (2018); published online Epub January (10.1016/j.mehy.2017.11.009).
J. P. Iyombe-Engembe, D. L. Ouellet, X. Barbeau, J. Rousseau, P. Chapdelaine, P. Lague, J. P. Tremblay, Efficient Restoration of the Dystrophin Gene Reading Frame and Protein Structure in DMD Myoblasts Using the CinDel Method. Molecular therapy. Nucleic acids 5, e283 (2016); published online EpubJan 26 (10.1038/mtna.2015.58).
B. Duchene, J. P. Iyombe-Engembe, J. Rousseau, J. P. Tremblay, D. L. Ouellet, From gRNA Identification to the Restoration of Dystrophin Expression: A Dystrophin Gene Correction Strategy for Duchenne Muscular Dystrophy Mutations Using the CRISPR-Induced Deletion Method. Methods in molecular biology 1687, 267-283 (2018)10.1007/978-1-4939-7374-3_19).
V. Kyrychenko, S. Kyrychenko, M. Tiburcy, J. M. Shelton, C. Long, J. W. Schneider, W. H. Zimmermann, R. Bassel-Duby, E. N. Olson, Functional correction of dystrophin actin binding domain mutations by genome editing. JCI insight 2, (2017); published online Epub September 21 (10.1172/jci.insight.95918).
T. Koo, N. B. Lu-Nguyen, A. Malerba, E. Kim, D. Kim, O. Cappellari, H. Y. Cho, G. Dickson, L. Popplewell, J. S. Kim, Functional Rescue of Dystrophin Deficiency in Mice Caused by Frameshift Mutations Using Campylobacter jejuni Cas9. Molecular therapy: the journal of the American Society of Gene Therapy 26, 1529-1538 (2018); published online Epub June 6 (10.1016/j.ymthe.2018.03.018).
L. Amoasii, J. C. W. Hildyard, H. Li, E. Sanchez-Ortiz, A. Mireault, D. Caballero, R. Harron, T. R. Stathopoulou, C. Massey, J. M. Shelton, R. Bassel-Duby, R. J. Piercy, E. N. Olson, Gene editing restores dystrophin expression in a canine model of Duchenne muscular dystrophy. Science 362, 86-91 (2018); published online Epub October 5 (10.1126/science.aau1549).
M. El Refaey, L. Xu, Y. Gao, B. D. Canan, T. M. A. Adesanya, S. C. Warner, K. Akagi, D. E. Symer, P. J. Mohler, J. Ma, P. M. L. Janssen, R. Han, In Vivo Genome Editing Restores Dystrophin Expression and Cardiac Function in Dystrophic Mice. Circulation research 121, 923-929 (2017); published online Epub September 29 (10.1161/CIRCRESAHA.117.310996).
D. G. Ousterout, A. M. Kabadi, P. 1. Thakore, W. H. Majoros, T. E. Reddy, C. A. Gersbach, Multiplex CRISPR-Cas9-based genome editing for correction of dystrophin mutations that cause Duchenne muscular dystrophy. Nature communications 6, 6244 (2015); published online Epub February 18 (10.1038/ncomms7244).
N. E. Bengtsson, J. K. Hall, G. L. Odom, M. P. Phelps, C. R. Andrus, R. D. Hawkins, S. D. Hauschka, J. R. Chamberlain, J. S. Chamberlain, Muscle-specific CRISPR-Cas9 dystrophin gene editing ameliorates pathophysiology in a mouse model for Duchenne muscular dystrophy. Nature communications 8, 14454 (2017); published online Epub February 14 (10.1038/ncomms14454).
C. Long, L. Amoasii, A. A. Mireault, J. R. McAnally, H. Li, E. Sanchez-Ortiz, S. Bhattacharyya, J. M. Shelton, R. Bassel-Duby, E. N. Olson, Postnatal genome editing partially restores dystrophin expression in a mouse model of muscular dystrophy. Science 351, 400-403 (2016); published online Epub January 22 (10.1126/science.aad5725).
H. L. Li, N. Fujimoto, N. Sasakawa, S. Shirai, T. Ohkame, T. Sakuma, M. Tanaka, N. Amano, A. Watanabe, H. Sakurai, T. Yamamoto, S. Yamanaka, A. Hotta, Precise correction of the dystrophin gene in duchenne muscular dystrophy patient induced pluripotent stem cells by TALEN and CRISPR-Cas9. Stem cell reports 4, 143-154 (2015); published online Epub January 13 (10.1016/j.stemcr.2014.10.013).
C. Long, J. R. McAnally, J. M. Shelton, A. A. Mireault, R. Bassel-Duby, E. N. Olson, Prevention of muscular dystrophy in mice by CRISPR-Cas9-mediated editing of germline DNA. Science 345, 1184-1188 (2014); published online Epub September 5 (10.1126/science.1254445).
M. Ifuku, K. A. Iwabuchi, M. Tanaka, M. S. Y. Lung, A. Hotta, Restoration of Dystrophin Protein Expression by Exon Skipping Utilizing CRISPR-Cas9 in Myoblasts Derived from DMD Patient iPS Cells. Methods in molecular biology 1828, 191-217 (2018)10.1007/978-1-4939-8651-4_12).
C. S. Young, M. R. Hicks, N. V. Ermolova, H. Nakano, M. Jan, S. Younesi, S. Karumbayaram, C. Kumagai-Cresse, D. Wang, J. A. Zack, D. B. Kohn, A. Nakano, S. F. Nelson, M. C. Miceli, M. J. Spencer, A. D. Pyle, A Single CRISPR-Cas9 Deletion Strategy that Targets the Majority of DMD Patients Restores Dystrophin Function in hiPSC-Derived Muscle Cells. Cell stem cell 18, 533-540 (2016); published online Epub April 7 (10.1016/j.stem.2016.01.021).
L. Amoasii, C. Long, H. Li, A. A. Mireault, J. M. Shelton, E. Sanchez-Ortiz, J. R. McAnally, S. Bhattacharyya, F. Schmidt, D. Grimm, S. D. Hauschka, R. Bassel-Duby, E. N. Olson, Single-cut genome editing restores dystrophin expression in a new mouse model of muscular dystrophy. Science translational medicine 9, (2017); published online Epub November 29 (10.1126/scitranslmed.aan8081).
WO 2017103624 A1=Dual AAV vector system for CRISPR-Cas9 mediated correction of human disease (Inventors: James M. Wilson, Lili Wang, Yang Yang).
Yang, Y. et al. A dual AAV system enables the Cas9-mediated correction of a metabolic liver disease in newborn mice. Nature biotechnology 34, 334-338, doi:10.1038/nbt.3469 (2016).
WO 2016097218 and WO 2016097219 A1=ADENO-ASSOCIATED VIRUS-MEDIATED CRISPR-Cas9 TREATMENT OF OCULAR DISEASE (Inventors: George Buchlis, Xavier Anguela, Katherine A. High).
WO2017197238A1=AAV split cas9 genome editing and transcriptional regulation. (Inventors: George M. Church, Wei Leong Chew.)
Truong, D. J. et al. Development of an intein-mediated split-Cas9 system for gene therapy. Nucleic acids research 43, 6450-6458, doi:10.1093/nar/gkv601 (2015).
Wright, A. V. et al. Rational design of a split-Cas9 enzyme complex. Proceedings of the National Academy of Sciences of the United States of America 112, 2984-2989, doi:10.1073/pnas.1501698112 (2015).
WO2016025469A1=Prevention of muscular dystrophy by CRISPR-Cas9-mediated gene editing. (Inventors: Eric N. Olson, Chengzu LONG, John R. MCANALLY, John M. SHELTON, Rhonda Bassel-Duby.)
WO2017193029A2=Crispr/cas-related methods and compositions for treating duchenne muscular dystrophy. (Inventors: David A. Bumcrot, Nicholas C. Huston, Joshua C. Tycko, Jacqueline Robinson-Hamm, Charles A. Gersbach).
Iyombe-Engembe, J. P. et al. Efficient Restoration of the Dystrophin Gene Reading Frame and Protein Structure in DMD Myoblasts Using the CinDel Method. Molecular therapy. Nucleic Acids 5, e283, doi:10.1038/mtna.2015.58 (2016).
WO2019036599.
US Patent Publication No. 2018/0265859.

1. A nucleic acid comprising:
(a) a nucleotide sequence encoding an RNA sequence comprising at least 90% identity to the sequence set forth in any one of SEQ ID NOs: 1-184;
(b) a nucleotide sequence encoding the RNA sequence set forth in any one of SEQ ID NOs: 1-184;
(c) a nucleotide sequence comprising an RNA sequence comprising at least 90% identity to the sequence set forth in any one of SEQ ID NOs: 1-184;
(d) a nucleotide sequence comprising the RNA sequence set forth in any one of SEQ ID NOs: 1-184; or
(e) a nucleotide sequence that specifically hybridizes to a target nucleic acid of the DMD gene comprising the nucleotide sequence set forth in any one of SEQ ID NOs: 185-276.

2. The nucleic acid of claim 1 further comprising a promoter sequence.

3. The nucleic acid of claim 2, wherein the promoter is any of U6, U7, tRNA, H1, minimal CMV, T7, EF1-alpha, minimal EF1-alpha, or a muscle-specific promoter.

4. The nucleic acid of claim 2, wherein the promoter is U6 or H1.

5. The nucleic acid of claim 3, wherein the muscle-specific promoter is unc45b, tMCK, minimal MCK, CK6, CK7, CK8, MHCK7, CK8e, SPC5-12, or CK1.

6. An adeno-associated virus comprising the nucleic acid of claim 1.

7. The adeno-associated virus of claim 6, wherein the virus lacks rep and cap genes.

8. The adeno-associated virus of claim 6, wherein the virus is a recombinant AAV (rAAV) or a self-complementary recombinant AAV (scAAV).

9. The adeno-associated virus of claim 6, wherein the virus is AAV1, AAV2, AAV3, AAV4, AAV5, AAV6, AAV7, AAV8, AAV9, AAV10, AAV11, AAV12, AAV13, AAVanc80, AAVrh.74, AAVrh.8, AAVrh.10, MyoAAV 1A, AAVMYO, or AAV-B1.

10. The adeno-associated virus of claim 6, wherein the virus is AAV1, AAV9 or AAVrh.74.

11. A nanoparticle, extracellular vesicle, or exosome comprising the nucleic acid of claim 1.

12. A composition comprising
the nucleic acid of claim 1;
and
a pharmaceutically acceptable carrier.

13. A method of correcting a mutation of the dystrophin (DMD) gene in a cell comprising contacting the cell with
(a) the nucleic acid of claim 1;
and
(b) a nucleic acid encoding a Cas9 enzyme or a functional fragment thereof.

14. A method of treating, ameliorating, and/or preventing a muscular dystrophy in a subject having a mutation in the dystrophin (DMD) gene comprising administering to the subject an effective amount of
(a) the nucleic acid of claim 1;
and
(b) a nucleic acid encoding a Cas9 enzyme or a functional fragment thereof.

15. The method of claim 14, wherein the muscular dystrophy is Duchenne's muscular dystrophy (DMD), Becker's muscular dystrophy (BMD), or intermediate muscular dystrophy (IMD).

16. The method of claim 14, wherein the mutation is a single- or multiple-exon duplication of the DMD gene.

17. The method of claim 16, wherein the single- or multiple-exon duplication is involving, surrounding, or affecting exon 2 or 3 of the DMD gene.

18. The method of claim 17, wherein the duplication is a duplication of exon(s) 2, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50, 2-51, 2-52, 2-53, 2-54, 2-55, 2-56, 2-57, 2-58, 2-59, 2-60, 2-61, 2-62, 2-63, 2-64, 2-65, 2-66, 2-67, 2-68, 2-69, 2-70, 2-71, 2-72, 2-73, 2-74, 2-75, 2-76, 2-77, 2-78, 2-79, 3, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-55, 3-56, 3-57, 3-58, 3-59, 3-60, 3-61, 3-62, 3-63, 3-64, 3-65, 3-66, 3-67, 3-68, 3-69, 3-70, 3-71, 3-72, 3-73, 3-74, 3-75, 3-76, 3-77, 3-78, or 3-79 of the DMD gene.

19-26. (canceled)

27. The method of claim 14 which results in increased expression of dystrophin protein in the subject.

28. The method of claim 14 which inhibits progression of dystrophic pathology in the subject.

29. The method of claim 14 which improves muscle function in the subject.

30. The method of claim 29 wherein the improvement in muscle function is an improvement in muscle strength.

31. The method of claim 29 wherein the improvement in muscle function is an improvement in stability in standing and walking.

32. (canceled)


SEQUENCE LISTING (281 sequences)

SEQ ID NO: 1 - RNA, 104 nt, S. Aureus
gaucauacaguauuugaacgacuguuuuaguacucuggaaacagaaucuacuaaaacaaggcaaaaugccguguuuaucucgucaacuuguuggcgagauuuuu

SEQ ID NO: 2 - RNA, 104 nt, S. Aureus
guucagauauuacaaaucugaggguuuuaguacucuggaaacagaaucuacuaaaacaaggcaaaaugccguguuuaucucgucaacuuguuggcgagauuuuu

SEQ ID NO: 3 - RNA, 103 nt, S. Aureus
gaucuugcucugucgcccacgcguuuuaguacucuggaaacagaaucuacuaaaacaaggcaaaaugccguguuuaucucgucaacuuguuggcgagauuuuu

SEQ ID NO: 4 - RNA, 103 nt, S. Aureus
ggcucacugcaaccuccaccucguuuuaguacucuggaaacagaaucuacuaaaacaaggcaaaaugccguguuuaucucgucaacuuguuggcgagauuuuu

SEQ ID NO: 5 - RNA, 103 nt, S. Aureus
gaggcugaggcaggagaaucauguuuuaguacucuggaaacagaaucuacuaaaacaaggcaaaaugccguguuuaucucgucaacuuguuggcgagauuuuu

SEQ ID NO: 6 - RNA, 103 nt, S. Aureus
gcuacuugggaggcugaggcagguuuuaguacucuggaaacagaaucuacuaaaacaaggcaaaaugccguguuuaucucgucaacuuguuggcgagauuuuu

SEQ ID NO: 7 - RNA, 104 nt, S. Aureus
gcugccucagccucccaaguagcguuuuaguacucuggaaacagaaucuacuaaaacaaggcaaaaugccguguuuaucucgucaacuuguuggcgagauuuuu

SEQ ID NO: 8 - RNA, 104 nt, S. Aureus
gauuuuuguauuuucaguagagaguuuuaguacucuggaaacagaaucuacuaaaacaaggcaaaaugccguguuuaucucgucaacuuguuggcgagauuuuu

SEQ ID NO: 9 - RNA, 104 nt, S. Aureus
gacaguuugggaggccagugaggguuuuaguacucuggaaacagaaucuacuaaaacaaggcaaaaugccguguuuaucucgucaacuuguuggcgagauuuuu

SEQ ID NO: 10 - RNA, 104 nt, S. Aureus
gccucacuggccucccaaacuguguuuuaguacucuggaaacagaaucuacuaaaacaaggcaaaaugccguguuuaucucgucaacuuguuggcgagauuuuu

SEQ ID NO: 11 - RNA, 104 nt, S. Aureus
gauuuuucagaaguaauuuuaauguuuuaguacucuggaaacagaaucuacuaaaacaaggcaaaaugccguguuuaucucgucaacuuguuggcgagauuuuu

SEQ ID NO: 12 - RNA, 104 nt, S. Aureus
gucauugauaaauggaauuaaacguuuuaguacucuggaaacagaaucuacuaaaacaaggcaaaaugccguguuuaucucgucaacuuguuggcgagauuuuu

SEQ ID NO: 13 - RNA, 104 nt, S. Aureus
gucauguuuaauuccauuuaucaguuuuaguacucuggaaacagaaucuacuaaaacaaggcaaaaugccguguuuaucucgucaacuuguuggcgagauuuuu

SEQ ID NO: 14 - RNA, 104 nt, S. Aureus
guaguauuacauucauugauaaaguuuuaguacucuggaaacagaaucuacuaaaacaaggcaaaaugccguguuuaucucgucaacuuguuggcgagauuuuu

SEQ ID NO: 15 - RNA, 104 nt, S. Aureus
gaaaguuacuuugguuguaaaauguuuuaguacucuggaaacagaaucuacuaaaacaaggcaaaaugccguguuuaucucgucaacuuguuggcgagauuuuu

SEQ ID NO: 16 - RNA, 104 nt, S. Aureus
guucaaaagaaaacauucacaaaguuuuaguacucuggaaacagaaucuacuaaaacaaggcaaaaugccguguuuaucucgucaacuuguuggcgagauuuuu

SEQ ID NO: 17 - RNA, 104 nt, S. Aureus
gaaauugugcauuuacccauuuuguuuuaguacucuggaaacagaaucuacuaaaacaaggcaaaaugccguguuuaucucgucaacuuguuggcgagauuuuu

SEQ ID NO: 18 - RNA, 104 nt, S. Aureus
guaaaugcacaauuuucuaagguguuuuaguacucuggaaacagaaucuacuaaaacaaggcaaaaugccguguuuaucucgucaacuuguuggcgagauuuuu

SEQ ID NO: 19 - RNA, 104 nt, S. Aureus
gaugacacuaugagagaaauaaaguuuuaguacucuggaaacagaaucuacuaaaacaaggcaaaaugccguguuuaucucgucaacuuguuggcgagauuuuu

SEQ ID NO: 20 - RNA, 104 nt, S. Aureus
gauauuauguucacucuuauuuaguuuuaguacucuggaaacagaaucuacuaaaacaaggcaaaaugccguguuuaucucgucaacuuguuggcgagauuuuu

SEQ ID NO: 21 - RNA, 104 nt, S. Aureus
guucugcugcuuacuccuuaaauguuuuaguacucuggaaacagaaucuacuaaaacaaggcaaaaugccguguuuaucucgucaacuuguuggcgagauuuuu

SEQ ID NO: 22 - RNA, 104 nt, S. Aureus
gaucuuugauucuucuugacaauguuuuaguacucuggaaacagaaucuacuaaaacaaggcaaaaugccguguuuaucucgucaacuuguuggcgagauuuuu

SEQ ID NO: 23 - RNA, 104 nt, S. Aureus
guaaagaaacucacauugucaagguuuuaguacucuggaaacagaaucuacuaaaacaaggcaaaaugccguguuuaucucgucaacuuguuggcgagauuuuu

SEQ ID NO: 24 - RNA, 104 nt, S. Aureus
gcaaugugaguuucuuuaaugacguuuuaguacucuggaaacagaaucuacuaaaacaaggcaaaaugccguguuuaucucgucaacuuguuggcgagauuuuu

SEQ ID NO: 25 - RNA, 104 nt, S. Aureus
guuucuuuaaugacaugaguccuguuuuaguacucuggaaacagaaucuacuaaaacaaggcaaaaugccguguuuaucucgucaacuuguuggcgagauuuuu

SEQ ID NO: 26 - RNA, 104 nt, S. Aureus
gcugcaaucuuccaucaaguagcguuuuaguacucuggaaacagaaucuacuaaaacaaggcaaaaugccguguuuaucucgucaacuuguuggcgagauuuuu

SEQ ID NO: 27 - RNA, 104 nt, S. Aureus
gcacccagcagaagaagauaugaguuuuaguacucuggaaacagaaucuacuaaaacaaggcaaaaugccguguuuaucucgucaacuuguuggcgagauuuuu

SEQ ID NO: 28 - RNA, 104 nt, S. Aureus
gaauucccucauaucuucuucugguuuuaguacucuggaaacagaaucuacuaaaacaaggcaaaaugccguguuuaucucgucaacuuguuggcgagauuuuu

SEQ ID NO: 29 - RNA, 104 nt, S. Aureus
gcaagaaaagcaauauaauugcuguuuuaguacucuggaaacagaaucuacuaaaacaaggcaaaaugccguguuuaucucgucaacuuguuggcgagauuuuu

SEQ ID NO: 30 - RNA, 104 nt, S. Aureus
gaauaugcucuaaacuauaguggguuuuaguacucuggaaacagaaucuacuaaaacaaggcaaaaugccguguuuaucucgucaacuuguuggcgagauuuuu

SEQ ID NO: 31 - RNA, 104 nt, S. Aureus
gaaauuaaguaaauauuaagaguguuuuaguacucuggaaacagaaucuacuaaaacaaggcaaaaugccguguuuaucucgucaacuuguuggcgagauuuuu

SEQ ID NO: 32 - RNA, 104 nt, S. Aureus
gauuugcaaauuaaguaaauauuguuuuaguacucuggaaacagaaucuacuaaaacaaggcaaaaugccguguuuaucucgucaacuuguuggcgagauuuuu

SEQ ID NO: 33 - RNA, 103 nt, S. Aureus
gagucacgcuauugauuaaaaaguuuuaguacucuggaaacagaaucuacuaaaacaaggcaaaaugccguguuuaucucgucaacuuguuggcgagauuuuu

SEQ ID NO: 34 - RNA, 104 nt, S. Aureus
gugguauugaacauucaauuaaaguuuuaguacucuggaaacagaaucuacuaaaacaaggcaaaaugccguguuuaucucgucaacuuguuggcgagauuuuu

SEQ ID NO: 35 - RNA, 104 nt, S. Aureus
guuaauuuagaaauucuuuuuaaguuuuaguacucuggaaacagaaucuacuaaaacaaggcaaaaugccguguuuaucucgucaacuuguuggcgagauuuuu

SEQ ID NO: 36 - RNA, 104 nt, S. Aureus
gaauaaaaaauaaaauuuauaacguuuuaguacucuggaaacagaaucuacuaaaacaaggcaaaaugccguguuuaucucgucaacuuguuggcgagauuuuu

SEQ ID NO: 37 - RNA, 104 nt, S. Aureus
gaaauugugcauuuauccauuuuguuuuaguacucuggaaacagaaucuacuaaaacaaggcaaaaugccguguuuaucucgucaacuuguuggcgagauuuuu

SEQ ID NO: 38 - RNA, 104 nt, S. Aureus; identical to SEQ ID NO: 16

SEQ ID NO: 39 - RNA, 104 nt, S. Aureus
guccacauuucaaauuauagcucguuuuaguacucuggaaacagaaucuacuaaaacaaggcaaaaugccguguuuaucucgucaacuuguuggcgagauuuuu

SEQ ID NO: 40 - RNA, 104 nt, S. Aureus
guucuagagcuauaauuugaaauguuuuaguacucuggaaacagaaucuacuaaaacaaggcaaaaugccguguuuaucucgucaacuuguuggcgagauuuuu

SEQ ID NO: 41 - RNA, 104 nt, S. Aureus
guuucuuaugugucauuuuaauuguuuuaguacucuggaaacagaaucuacuaaaacaaggcaaaaugccguguuuaucucgucaacuuguuggcgagauuuuu

SEQ ID NO: 42 - RNA, 104 nt, S. Aureus
guuuaaaacauauguaaagagguguuuuaguacucuggaaacagaaucuacuaaaacaaggcaaaaugccguguuuaucucgucaacuuguuggcgagauuuuu

SEQ ID NO: 43 - RNA, 102 nt, C. Jejuni
guuaucuuugcuucccagaacuguuuuagucccugaaaagggacuaaaauaaagaguuugcgggacucugcgggguuacaauccccuaaaaccgcuuuuuuu

SEQ ID NO: 44 - RNA, 103 nt, C. Jejuni
guaguuaucuuugcuucccagaaguuuuagucccugaaaagggacuaaaauaaagaguuugcgggacucugcgggguuacaauccccuaaaaccgcuuuuuuu

SEQ ID NO: 45 - RNA, 103 nt, C. Jejuni
gauuauuccuuccuuugcagcauguuuuagucccugaaaagggacuaaaauaaagaguuugcgggacucugcgggguuacaauccccuaaaaccgcuuuuuuu

SEQ ID NO: 46 - RNA, 103 nt, C. Jejuni
gaugacagcugagaguuucuuuuguuuuagucccugaaaagggacuaaaauaaagaguuugcgggacucugcgggguuacaauccccuaaaaccgcuuuuuuu

SEQ ID NO: 47 - RNA, 103 nt, C. Jejuni
gaguaucuauucauaacuggcgaguuuuagucccugaaaagggacuaaaauaaagaguuugcgggacucugcgggguuacaauccccuaaaaccgcuuuuuuu

SEQ ID NO: 48 - RNA, 103 nt, C. Jejuni
gaguauuuauucgccaguuaugaguuuuagucccugaaaagggacuaaaauaaagaguuugcgggacucugcgggguuacaauccccuaaaaccgcuuuuuuu

SEQ ID NO: 49 - RNA, 103 nt, C. Jejuni
gucagaggagacuauuuuuuuauguuuuagucccugaaaagggacuaaaauaaagaguuugcgggacucugcgggguuacaauccccuaaaaccgcuuuuuuu

SEQ ID NO: 50 - RNA, 103 nt, C. Jejuni
gauaugaccaaacccauagucguguuuuagucccugaaaagggacuaaaauaaagaguuugcgggacucugcgggguuacaauccccuaaaaccgcuuuuuuu

SEQ ID NO: 51 - RNA, 103 nt, C. Jejuni
guugcagugagccgagaucauggguuuuagucccugaaaagggacuaaaauaaagaguuugcgggacucugcgggguuacaauccccuaaaaccgcuuuuuuu

SEQ ID NO: 52 - RNA, 102 nt, C. Jejuni
ggagguugcagugagccgagauguuuuagucccugaaaagggacuaaaauaaagaguuugcgggacucugcgggguuacaauccccuaaaaccgcuuuuuuu

SEQ ID NO: 53 - RNA, 103 nt, C. Jejuni
gcaaaaauuagccaggcauggugguuuuagucccugaaaagggacuaaaauaaagaguuugcgggacucugcgggguuacaauccccuaaaaccgcuuuuuuu

SEQ ID NO: 54 - RNA, 103 nt, C. Jejuni
gauggcgaaaccccaucucuacuguuuuagucccugaaaagggacuaaaauaaagaguuugcgggacucugcgggguuacaauccccuaaaaccgcuuuuuuu

SEQ ID NO: 55 - RNA, 103 nt, C. Jejuni
guuuaauuccauuuaucaaugaaguuuuagucccugaaaagggacuaaaauaaagaguuugcgggacucugcgggguuacaauccccuaaaaccgcuuuuuuu

SEQ ID NO: 56 - RNA, 103 nt, C. Jejuni
gaugaauguaauacuaaauguaaguuuuagucccugaaaagggacuaaaauaaagaguuugcgggacucugcgggguuacaauccccuaaaaccgcuuuuuuu

SEQ ID NO: 57 - RNA, 102 nt, C. Jejuni
guaauacuaaauguaaaaaaacguuuuagucccugaaaagggacuaaaauaaagaguuugcgggacucugcgggguuacaauccccuaaaaccgcuuuuuuu

SEQ ID NO: 58 - RNA, 103 nt, C. Jejuni
gagaaaacauucacaaaauggguguuuuagucccugaaaagggacuaaaauaaagaguuugcgggacucugcgggguuacaauccccuaaaaccgcuuuuuuu

SEQ ID NO: 59 - RNA, 102 nt, C. Jejuni
guugugaaauuuucaaaauggaguuuuagucccugaaaagggacuaaaauaaagaguuugcgggacucugcgggguuacaauccccuaaaaccgcuuuuuuu

SEQ ID NO: 60 - RNA, 103 nt, C. Jejuni
gauaaaacggauuuuuaagauacguuuuagucccugaaaagggacuaaaauaaagaguuugcgggacucugcgggguuacaauccccuaaaaccgcuuuuuuu

SEQ ID NO: 61 - RNA, 103 nt, C. Jejuni
gagagaaauaaaacggauuuuuaguuuuagucccugaaaagggacuaaaauaaagaguuugcgggacucugcgggguuacaauccccuaaaaccgcuuuuuuu

SEQ ID NO: 62 - RNA, 103 nt, C. Jejuni
gugagagaaauaaaacggauuuuguuuuagucccugaaaagggacuaaaauaaagaguuugcgggacucugcgggguuacaauccccuaaaaccgcuuuuuuu

SEQ ID NO: 63 - RNA, 103 nt, C. Jejuni
gcagauuugcacagcuaaaauaaguuuuagucccugaaaagggacuaaaauaaagaguuugcgggacucugcgggguuacaauccccuaaaaccgcuuuuuuu

SEQ ID NO: 64 - RNA, 103 nt, C. Jejuni
gagagugaacauaauauuuccagguuuuagucccugaaaagggacuaaaauaaagaguuugcgggacucugcgggguuacaauccccuaaaaccgcuuuuuuu

SEQ ID NO: 65 - RNA, 103 nt, C. Jejuni
gaguaagcagcagaagauauggcguuuuagucccugaaaagggacuaaaauaaagaguuugcgggacucugcgggguuacaauccccuaaaaccgcuuuuuuu

SEQ ID NO: 66 - RNA, 103 nt, C. Jejuni
guaagcagcagaagauauggcaaguuuuagucccugaaaagggacuaaaauaaagaguuugcgggacucugcgggguuacaauccccuaaaaccgcuuuuuuu

SEQ ID NO: 67 - RNA, 103 nt, C. Jejuni
gucacauugucaagaagaaucaaguuuuagucccugaaaagggacuaaaauaaagaguuugcgggacucugcgggguuacaauccccuaaaaccgcuuuuuuu

SEQ ID NO: 68 - RNA, 103 nt, C. Jejuni
guaugcucuaaacuauaguggcuguuuuagucccugaaaagggacuaaaauaaagaguuugcgggacucugcgggguuacaauccccuaaaaccgcuuuuuuu

SEQ ID NO: 69 - RNA, 103 nt, C. Jejuni
gaauaugcucuaaacuauaguggguuuuagucccugaaaagggacuaaaauaaagaguuugcgggacucugcgggguuacaauccccuaaaaccgcuuuuuuu

SEQ ID NO: 70 - RNA, 103 nt, C. Jejuni
gucuuguuuuugugcaggcuucaguuuuagucccugaaaagggacuaaaauaaagaguuugcgggacucugcgggguuacaauccccuaaaaccgcuuuuuuu

SEQ ID NO: 71 - RNA, 103 nt, C. Jejuni
gaauauuaagaguauggauugaaguuuuagucccugaaaagggacuaaaauaaagaguuugcgggacucugcgggguuacaauccccuaaaaccgcuuuuuuu

SEQ ID NO: 72 - RNA, 103 nt, C. Jejuni
gacucuuaauauuuacuuaauuuguuuuagucccugaaaagggacuaaaauaaagaguuugcgggacucugcgggguuacaauccccuaaaaccgcuuuuuuu

SEQ ID NO: 73 - RNA, 102 nt, C. Jejuni
gcgugacucuagaugauuauagguuuuagucccugaaaagggacuaaaauaaagaguuugcgggacucugcgggguuacaauccccuaaaaccgcuuuuuuu

SEQ ID NO: 74 - RNA, 103 nt, C. Jejuni
gcuaaauuaauagauauguucagguuuuagucccugaaaagggacuaaaauaaagaguuugcgggacucugcgggguuacaauccccuaaaaccgcuuuuuuu

SEQ ID NO: 75 - RNA, 103 nt, C. Jejuni
gagaaauucuuuuuaauugaaugguuuuagucccugaaaagggacuaaaauaaagaguuugcgggacucugcgggguuacaauccccuaaaaccgcuuuuuuu

SEQ ID NO: 76 - RNA, 103 nt, C. Jejuni
gagaaaacauucacaaaauggauguuuuagucccugaaaagggacuaaaauaaagaguuugcgggacucugcgggguuacaauccccuaaaaccgcuuuuuuu

SEQ ID NO: 77 - RNA, 103 nt, C. Jejuni
guagaaucaauuucaagaagucaguuuuagucccugaaaagggacuaaaauaaagaguuugcgggacucugcgggguuacaauccccuaaaaccgcuuuuuuu

SEQ ID NO: 78 - RNA, 103 nt, C. Jejuni
gagacuugcagauccaaaauuaaguuuuagucccugaaaagggacuaaaauaaagaguuugcgggacucugcgggguuacaauccccuaaaaccgcuuuuuuu

SEQ ID NO: 79 - RNA, 104 nt, S. Aureus
gagaaaguuaacuuuaaacuuagguuuuaguacucuggaaacagaaucuacuaaaacaaggcaaaaugccguguuuaucucgucaacuuguuggcgagauuuuu

SEQ ID NO: 80 - RNA, 104 nt, S. Aureus
gaacauaguuuuaugacaucuagguuuuaguacucuggaaacagaaucuacuaaaacaaggcaaaaugccguguuuaucucgucaacuuguuggcgagauuuuu

SEQ ID NO: 81 - RNA, 104 nt, S. Aureus
gaucauacgagguugcuuuacuaguuuuaguacucuggaaacagaaucuacuaaaacaaggcaaaaugccguguuuaucucgucaacuuguuggcgagauuuuu

SEQ ID NO: 82 - RNA, 104 nt, S. Aureus
gaguuauuuuacaaggaaccuguguuuuaguacucuggaaacagaaucuacuaaaacaaggcaaaaugccguguuuaucucgucaacuuguuggcgagauuuuu

SEQ ID NO: 83 - RNA, 104 nt, S. Aureus
gaaccuguaugaguuuauacaugguuuuaguacucuggaaacagaaucuacuaaaacaaggcaaaaugccguguuuaucucgucaacuuguuggcgagauuuuu

SEQ ID NO: 84 - RNA, 104 nt, S. Aureus
guauaucaaucauguaucuucacguuuuaguacucuggaaacagaaucuacuaaaacaaggcaaaaugccguguuuaucucgucaacuuguuggcgagauuuuu

SEQ ID NO: 85 - RNA, 104 nt, S. Aureus
gugggggcuuaaacucuacuuuuguuuuaguacucuggaaacagaaucuacuaaaacaaggcaaaaugccguguuuaucucgucaacuuguuggcgagauuuuu

SEQ ID NO: 86 - RNA, 104 nt, S. Aureus
gcuuagauugcuauucuaaaaagguuuuaguacucuggaaacagaaucuacuaaaacaaggcaaaaugccguguuuaucucgucaacuuguuggcgagauuuuu

SEQ ID NO: 87 - RNA, 104 nt, S. Aureus
gagagacgaaggcuaugauuuagguuuuaguacucuggaaacagaaucuacuaaaacaaggcaaaaugccguguuuaucucgucaacuuguuggcgagauuuuu

SEQ ID NO: 88 - RNA, 104 nt, S. Aureus
gacaggaggcaggcugggguagaguuuuaguacucuggaaacagaaucuacuaaaacaaggcaaaaugccguguuuaucucgucaacuuguuggcgagauuuuu

SEQ ID NO: 89 - RNA, 104 nt, S. Aureus
guuaaguguuacaggaggcaggcguuuuaguacucuggaaacagaaucuacuaaaacaaggcaaaaugccguguuuaucucgucaacuuguuggcgagauuuuu

SEQ ID NO: 90 - RNA, 104 nt, S. Aureus
gaucugacaauauauaccgagaaguuuuaguacucuggaaacagaaucuacuaaaacaaggcaaaaugccguguuuaucucgucaacuuguuggcgagauuuuu

SEQ ID NO: 91 - RNA, 104 nt, S. Aureus
gcgguauauauugucagauaucuguuuuaguacucuggaaacagaaucuacuaaaacaaggcaaaaugccguguuuaucucgucaacuuguuggcgagauuuuu

SEQ ID NO: 92 - RNA, 104 nt, S. Aureus
gaucaagguuucuggcagacaguguuuuaguacucuggaaacagaaucuacuaaaacaaggcaaaaugccguguuuaucucgucaacuuguuggcgagauuuuu

SEQ ID NO: 93 - RNA, 23 nt, S. Aureus
gaucauacaguauuugaacgacu

SEQ ID NO: 94 - RNA, 23 nt, S. Aureus
guucagauauuacaaaucugagg

SEQ ID NO: 95 - RNA, 22 nt, S. Aureus
gaucuugcucugucgcccacgc

SEQ ID NO: 96 - RNA, 22 nt, S. Aureus
ggcucacugcaaccuccaccuc

SEQ ID NO: 97 - RNA, 22 nt, S. Aureus
gaggcugaggcaggagaaucau

SEQ ID NO: 98 - RNA, 22 nt, S. Aureus
gcuacuugggaggcugaggcag

SEQ ID NO: 99 - RNA, 23 nt, S. Aureus
gcugccucagccucccaaguagc

SEQ ID NO: 100 - RNA, 23 nt, S. Aureus
gauuuuuguauuuucaguagaga

SEQ ID NO: 101 - RNA, 23 nt, S. Aureus
gacaguuugggaggccagugagg

SEQ ID NO: 102 - RNA, 23 nt, S. Aureus
gccucacuggccucccaaacugu

SEQ ID NO: 103 - RNA, 23 nt, S. Aureus
gauuuuucagaaguaauuuuaau

SEQ ID NO: 104 - RNA, 23 nt, S. Aureus
gucauugauaaauggaauuaaac

SEQ ID NO: 105 - RNA, 23 nt, S. Aureus
gucauguuuaauuccauuuauca

SEQ ID NO: 106 - RNA, 23 nt, S. Aureus
guaguauuacauucauugauaaa

SEQ ID NO: 107 - RNA, 23 nt, S. Aureus
gaaaguuacuuugguuguaaaau

SEQ ID NO: 108 - RNA, 23 nt, S. Aureus
guucaaaagaaaacauucacaaa

SEQ ID NO: 109 - RNA, 23 nt, S. Aureus
gaaauugugcauuuacccauuuu

SEQ ID NO: 110 - RNA, 23 nt, S. Aureus
guaaaugcacaauuuucuaaggu

SEQ ID NO: 111 - RNA, 23 nt, S. Aureus
gaugacacuaugagagaaauaaa

SEQ ID NO: 112 - RNA, 23 nt, S. Aureus
gauauuauguucacucuuauuua

SEQ ID NO: 113 - RNA, 23 nt, S. Aureus
guucugcugcuuacuccuuaaau

SEQ ID NO: 114 - RNA, 23 nt, S. Aureus
gaucuuugauucuucuugacaau

SEQ ID NO: 115 - RNA, 23 nt, S. Aureus
guaaagaaacucacauugucaag

SEQ ID NO: 116 - RNA, 23 nt, S. Aureus
gcaaugugaguuucuuuaaugac

SEQ ID NO: 117 - RNA, 23 nt, S. Aureus
guuucuuuaaugacaugaguccu

SEQ ID NO: 118 - RNA, 23 nt, S. Aureus
gcugcaaucuuccaucaaguagc

SEQ ID NO: 119 - RNA, 23 nt, S. Aureus
gcacccagcagaagaagauauga

SEQ ID NO: 120 - RNA, 23 nt, S. Aureus
gaauucccucauaucuucuucug

SEQ ID NO: 121 - RNA, 23 nt, S. Aureus
gcaagaaaagcaauauaauugcu

SEQ ID NO: 122 - RNA, 23 nt, S. Aureus
gaauaugcucuaaacuauagugg

SEQ ID NO: 123 - RNA, 23 nt, S. Aureus
gaaauuaaguaaauauuaagagu

SEQ ID NO: 124 - RNA, 23 nt, S. Aureus
gauuugcaaauuaaguaaauauu

SEQ ID NO: 125 - RNA, 22 nt, S. Aureus
gagucacgcuauugauuaaaaa

SEQ ID NO: 126 - RNA, 23 nt, S. Aureus
gugguauugaacauucaauuaaa

SEQ ID NO: 127 - RNA, 23 nt, S. Aureus
guuaauuuagaaauucuuuuuaa

SEQ ID NO: 128 - RNA, 23 nt, S. Aureus
gaauaaaaaauaaaauuuauaac

SEQ ID NO: 129 - RNA, 23 nt, S. Aureus
gaaauugugcauuuauccauuuu

SEQ ID NO: 130 - RNA, 23 nt, S. Aureus
guucaaaagaaaacauucacaaa

SEQ ID NO: 131 - RNA, 23 nt, S. Aureus
guccacauuucaaauuauagcuc

SEQ ID NO: 132 - RNA, 23 nt, S. Aureus
guucuagagcuauaauuugaaau

SEQ ID NO: 133 - RNA, 23 nt, S. Aureus
guuucuuaugugucauuuuaauu

SEQ ID NO: 134 - RNA, 23 nt, S. Aureus
guuuaaaacauauguaaagaggu

SEQ ID NO: 135 - RNA, 22 nt, C. Jejuni
guuaucuuugcuucccagaacu

SEQ ID NO: 136 - RNA, 23 nt, C. Jejuni
guaguuaucuuugcuucccagaa

SEQ ID NO: 137 - RNA, 23 nt, C. Jejuni
gauuauuccuuccuuugcagcau

SEQ ID NO: 138 - RNA, 23 nt, C. Jejuni
gaugacagcugagaguuucuuuu

SEQ ID NO: 139 - RNA, 23 nt, C. Jejuni
gaguaucuauucauaacuggcga

SEQ ID NO: 140 - RNA, 23 nt, C. Jejuni
gaguauuuauucgccaguuauga

SEQ ID NO: 141 - RNA, 23 nt, C. Jejuni
gucagaggagacuauuuuuuuau

SEQ ID NO: 142 - RNA, 23 nt, C. Jejuni
gauaugaccaaacccauagucgu

SEQ ID NO: 143 - RNA, 23 nt, C. Jejuni
guugcagugagccgagaucaugg

SEQ ID NO: 144 - RNA, 22 nt, C. Jejuni
ggagguugcagugagccgagau

SEQ ID NO: 145 - RNA, 23 nt, C. Jejuni
gcaaaaauuagccaggcauggug

SEQ ID NO: 146 - RNA, 23 nt, C. Jejuni
gauggcgaaaccccaucucuacu

SEQ ID NO: 147 - RNA, 23 nt, C. Jejuni
guuuaauuccauuuaucaaugaa

SEQ ID NO: 148 - RNA, 23 nt, C. Jejuni
gaugaauguaauacuaaauguaa

SEQ ID NO: 149 - RNA, 22 nt, C. Jejuni
guaauacuaaauguaaaaaaac

SEQ ID NO: 150 - RNA, 23 nt, C. Jejuni
gagaaaacauucacaaaaugggu

SEQ ID NO: 151 - RNA, 22 nt, C. Jejuni
guugugaaauuuucaaaaugga

SEQ ID NO: 152 - RNA, 23 nt, C. Jejuni
gauaaaacggauuuuuaagauac

SEQ ID NO: 153 - RNA, 23 nt, C. Jejuni
gagagaaauaaaacggauuuuua

SEQ ID NO: 154 - RNA, 23 nt, C. Jejuni
gugagagaaauaaaacggauuuu

SEQ ID NO: 155 - RNA, 23 nt, C. Jejuni
gcagauuugcacagcuaaaauaa

SEQ ID NO: 156 - RNA, 23 nt, C. Jejuni
gagagugaacauaauauuuccag

SEQ ID NO: 157 - RNA, 23 nt, C. Jejuni
gaguaagcagcagaagauauggc

SEQ ID NO: 158 - RNA, 23 nt, C. Jejuni
guaagcagcagaagauauggcaa

SEQ ID NO: 159 - RNA, 23 nt, C. Jejuni
gucacauugucaagaagaaucaa

SEQ ID NO: 160 - RNA, 23 nt, C. Jejuni
guaugcucuaaacuauaguggcu

SEQ ID NO: 161 - RNA, 23 nt, C. Jejuni
gaauaugcucuaaacuauagugg

SEQ ID NO: 162 - RNA, 23 nt, C. Jejuni
gucuuguuuuugugcaggcuuca

SEQ ID NO: 163 - RNA, 23 nt, C. Jejuni
gaauauuaagaguauggauugaa

SEQ ID NO: 164 - RNA, 23 nt, C. Jejuni
gacucuuaauauuuacuuaauuu

SEQ ID NO: 165 - RNA, 22 nt, C. Jejuni
gcgugacucuagaugauuauag

SEQ ID NO: 166 - RNA, 23 nt, C. Jejuni
gcuaaauuaauagauauguucag

SEQ ID NO: 167 - RNA, 23 nt, C. Jejuni
gagaaauucuuuuuaauugaaug

SEQ ID NO: 168 - RNA, 23 nt, C. Jejuni
gagaaaacauucacaaaauggau

SEQ ID NO: 169 - RNA, 23 nt, C. Jejuni
guagaaucaauuucaagaaguca

SEQ ID NO: 170 - RNA, 23 nt, C. Jejuni
gagacuugcagauccaaaauuaa

SEQ ID NO: 171 - RNA, 23 nt, S. Aureus
gagaaaguuaacuuuaaacuuag

SEQ ID NO: 172 - RNA, 23 nt, S. Aureus
gaacauaguuuuaugacaucuag

SEQ ID NO: 173 - RNA, 23 nt, S. Aureus
gaucauacgagguugcuuuacua

SEQ ID NO: 174 - RNA, 23 nt, S. Aureus
gaguuauuuuacaaggaaccugu

SEQ ID NO: 175 - RNA, 23 nt, S. Aureus
gaaccuguaugaguuuauacaug

SEQ ID NO: 176 - RNA, 23 nt, S. Aureus
guauaucaaucauguaucuucac

SEQ ID NO: 177 - RNA, 23 nt, S. Aureus
gugggggcuuaaacucuacuuuu

SEQ ID NO: 178 - RNA, 23 nt, S. Aureus
gcuuagauugcuauucuaaaaag

SEQ ID NO: 179 - RNA, 23 nt, S. Aureus
gagagacgaaggcuaugauuuag

SEQ ID NO: 180 - RNA, 23 nt, S. Aureus
gacaggaggcaggcugggguaga

SEQ ID NO: 181 - RNA, 23 nt, S. Aureus
guuaaguguuacaggaggcaggc

SEQ ID NO: 182 - RNA, 23 nt, S. Aureus
gaucugacaauauauaccgagaa

SEQ ID NO: 183 - RNA, 23 nt, S. Aureus
gcgguauauauugucagauaucu

SEQ ID NO: 184 - RNA, 23 nt, S. Aureus
gaucaagguuucuggcagacagu

SEQ ID NO: 185 - DNA, 28 bp, S. Aureus
atcatacagtatttgaacgactatgggt

SEQ ID NO: 186 - DNA, 28 bp, S. Aureus
ttcagatattacaaatctgaggcgggat

SEQ ID NO: 187 - DNA, 28 bp, S. Aureus
gatcttgctctgtcgcccacgctggagt

SEQ ID NO: 188 - DNA, 28 bp, S. Aureus
ggctcactgcaacctccacctcctggat

SEQ ID NO: 189 - DNA, 28 bp, S. Aureus
attcaaatgattctcctgcctcagcctc

SEQ ID NO: 190 - DNA, 28 bp, S. Aureus
attctcctgcctcagcctcccaagtagc

SEQ ID NO: 191 - DNA, 28 bp, S. Aureus
ctgcctcagcctcccaagtagctgggat

SEQ ID NO: 192 - DNA, 28 bp, S. Aureus
atttttgtattttcagtagagatggggt

SEQ ID NO: 193 - DNA, 28 bp, S. Aureus
atccgtcctcactggcctcccaaactgt

SEQ ID NO: 194 - DNA, 28 bp, S. Aureus
cctcactggcctcccaaactgttgggat

SEQ ID NO: 195 - DNA, 28 bp, S. Aureus
atttttcagaagtaattttaatttggat

SEQ ID NO: 196 - DNA, 28 bp, S. Aureus
actcatgtttaattccatttatcaatga

SEQ ID NO: 197 - DNA, 28 bp, S. Aureus
tcatgtttaattccatttatcaatgaat

SEQ ID NO: 198 - DNA, 28 bp, S. Aureus
attccatttatcaatgaatgtaatacta

SEQ ID NO: 199 - DNA, 28 bp, S. Aureus
aaagttactttggttgtaaaatatgaat

SEQ ID NO: 200 - DNA, 28 bp, S. Aureus
ttcaaaagaaaacattcacaaaatgggt

SEQ ID NO: 201 - DNA, 28 bp, S. Aureus
attcacaaaatgggtaaatgcacaattt

SEQ ID NO: 202 - DNA, 28 bp, S. Aureus
taaatgcacaattttctaaggtaagaat

SEQ ID NO: 203 - DNA, 28 bp, S. Aureus
atccgttttatttctctcatagtgtcat

SEQ ID NO: 204 - DNA, 28 bp, S. Aureus
atattatgttcactcttatttaaggagt

SEQ ID NO: 205 - DNA, 28 bp, S. Aureus
actcttatttaaggagtaagcagcagaa

SEQ ID NO: 206 - DNA, 28 bp, S. Aureus
atctttgattcttcttgacaatgtgagt

SEQ ID NO: 207 - DNA, 28 bp, S. Aureus
attcttcttgacaatgtgagtttcttta

SEQ ID NO: 208 - DNA, 28 bp, S. Aureus
caatgtgagtttctttaatgacatgagt

SEQ ID NO: 209 - DNA, 28 bp, S. Aureus
tttctttaatgacatgagtcctcagagt

SEQ ID NO: 210 - DNA, 28 bp, S. Aureus
ctgcaatcttccatcaagtagctagaat

SEQ ID NO: 211 - DNA, 28 bp, S. Aureus
cacccagcagaagaagatatgagggaat

SEQ ID NO: 212 - DNA, 28 bp, S. Aureus
acccagcagaagaagatatgagggaatt

SEQ ID NO: 213 - DNA, 28 bp, S. Aureus
caagaaaagcaatataattgctttgggt

SEQ ID NO: 214 - DNA, 28 bp, S. Aureus
attcagccactatagtttagagcatatt

SEQ ID NO: 215 - DNA, 28 bp, S. Aureus
atccatactcttaatatttacttaattt

SEQ ID NO: 216 - DNA, 28 bp, S. Aureus
actcttaatatttacttaatttgcaaat

SEQ ID NO: 217 - DNA, 28 bp, S. Aureus
actcaatttttaatcaatagcgtgactc

SEQ ID NO: 218 - DNA, 28 bp, S. Aureus
attctttttaattgaatgttcaatacca

SEQ ID NO: 219 - DNA, 28 bp, S. Aureus
ttaatttagaaattctttttaattgaat

SEQ ID NO: 220 - DNA, 28 bp, S. Aureus
atcctagttataaattttattttttatt

SEQ ID NO: 221 - DNA, 28 bp, S. Aureus
attcacaaaatggataaatgcacaattt

SEQ ID NO: 222 - DNA, 28 bp, S. Aureus
ttcaaaagaaaacattcacaaaatggat

SEQ ID NO: 223 - DNA, 28 bp, S. Aureus
attctagagctataatttgaaatgtgga

SEQ ID NO: 224 - DNA, 28 bp, S. Aureus
ttctagagctataatttgaaatgtggat

SEQ ID NO: 225 - DNA, 28 bp, S. Aureus
tttcttatgtgtcattttaattttggat

SEQ ID NO: 226 - DNA, 28 bp, S. Aureus
tttaaaacatatgtaaagaggtgggaat

SEQ ID NO: 227 - DNA, 30 bp, C. Jejuni
gtgtgctaagttctgggaagcaaagataac

SEQ ID NO: 228 - DNA, 30 bp, C. Jejuni
gtgctaagttctgggaagcaaagataacta

SEQ ID NO: 229 - DNA, 30 bp, C. Jejuni
gtatgtagatgctgcaaaggaaggaataat

SEQ ID NO: 230 - DNA, 30 bp, C. Jejuni
atgacagctgagagtttcttttaattatac

SEQ ID NO: 231 - DNA, 30 bp, C. Jejuni
agtatctattcataactggcgaataaatac

SEQ ID NO: 232 - DNA, 30 bp, C. Jejuni
gtatctattcataactggcgaataaatact

SEQ ID NO: 233 - DNA, 30 bp, C. Jejuni
tcagaggagactatttttttataatcatac

SEQ ID NO: 234 - DNA, 30 bp, C. Jejuni
gtatttgaacgactatgggtttggtcatat

SEQ ID NO: 235 - DNA, 30 bp, C. Jejuni
gtgcagtgccatgatctcggctcactgcaa

SEQ ID NO: 236 - DNA, 30 bp, C. Jejuni
gtgccatgatctcggctcactgcaacctcc

SEQ ID NO: 237 - DNA, 30 bp, C. Jejuni
gtgcatgccaccatgcctggctaatttttg

SEQ ID NO: 238 - DNA, 30 bp, C. Jejuni
gtattttcagtagagatggggtttcgccat

SEQ ID NO: 239 - DNA, 30 bp, C. Jejuni
tttaattccatttatcaatgaatgtaatac

SEQ ID NO: 240 - DNA, 30 bp, C. Jejuni
atgaatgtaatactaaatgtaaaaaaacac

SEQ ID NO: 241 - DNA, 30 bp, C. Jejuni
gtaatactaaatgtaaaaaaacactaacac

SEQ ID NO: 242 - DNA, 30 bp, C. Jejuni
agaaaacattcacaaaatgggtaaatgcac

SEQ ID NO: 243 - DNA, 30 bp, C. Jejuni
gttgtgaaattttcaaaatggactatgtac

SEQ ID NO: 244 - DNA, 30 bp, C. Jejuni
gtacctgtgtatcttaaaaatccgttttat

SEQ ID NO: 245 - DNA, 30 bp, C. Jejuni
gtgtatcttaaaaatccgttttatttctct

SEQ ID NO: 246 - DNA, 30 bp, C. Jejuni
gtatcttaaaaatccgttttatttctctca

SEQ ID NO: 247 - DNA, 30 bp, C. Jejuni
gtgtcattttattttagctgtgcaaatctg

SEQ ID NO: 248 - DNA, 30 bp, C. Jejuni
gtgcaaatctggaaatattatgttcactct

SEQ ID NO: 249 - DNA, 30 bp, C. Jejuni
agtaagcagcagaagatatggcaaagatac

SEQ ID NO: 250 - DNA, 30 bp, C. Jejuni
taagcagcagaagatatggcaaagatacac

SEQ ID NO: 251 - DNA, 30 bp, C. Jejuni
gtatatctttgattcttcttgacaatgtga

SEQ ID NO: 252 - DNA, 30 bp, C. Jejuni
gtgtattcagccactatagtttagagcata

SEQ ID NO: 253 - DNA, 30 bp, C. Jejuni
gtattcagccactatagtttagagcatatt

SEQ ID NO: 254 - DNA, 30 bp, C. Jejuni
tcttgtttttgtgcaggcttcaatccatac

SEQ ID NO: 255 - DNA, 30 bp, C. Jejuni
gtgcaggcttcaatccatactcttaatatt

SEQ ID NO: 256 - DNA, 30 bp, C. Jejuni
actcttaatatttacttaatttgcaaatac

SEQ ID NO: 257 - DNA, 30 bp, C. Jejuni
gcgtgactctagatgattataggtggacac

SEQ ID NO: 258 - DNA, 30 bp, C. Jejuni
gtatggacctgaacatatctattaatttag

SEQ ID NO: 259 - DNA, 30 bp, C. Jejuni
agaaattctttttaattgaatgttcaatac

SEQ ID NO: 260 - DNA, 30 bp, C. Jejuni
agaaaacattcacaaaatggataaatgcac

SEQ ID NO: 261 - DNA, 30 bp, C. Jejuni
gtattgattgacttcttgaaattgattcta

SEQ ID NO: 262 - DNA, 30 bp, C. Jejuni
gtgtcattttaattttggatctgcaagtct

SEQ ID NO: 263 - DNA, 28 bp, S. Aureus
agaaagttaactttaaacttagtagaat

SEQ ID NO: 264 - DNA, 28 bp, S. Aureus
aacatagttttatgacatctagtagaat

SEQ ID NO: 265 - DNA, 28 bp, S. Aureus
attccttagtaaagcaacctcgtatgat

SEQ ID NO: 266 - DNA, 28 bp, S. Aureus
agttattttacaaggaacctgtatgagt

SEQ ID NO: 267 - DNA, 28 bp, S. Aureus
aacctgtatgagtttatacatgtgggat

SEQ ID NO: 268 - DNA, 28 bp, S. Aureus
attcctgtgaagatacatgattgatata

SEQ ID NO: 269 - DNA, 28 bp, S. Aureus
attctaaaaagtagagtttaagccccca

SEQ ID NO: 270 - DNA, 28 bp, S. Aureus
cttagattgctattctaaaaagtagagt

SEQ ID NO: 271 - DNA, 28 bp, S. Aureus
attctgctaaatcatagccttcgtctct

SEQ ID NO: 272 - DNA, 28 bp, S. Aureus
atcctatctaccccagcctgcctcctgt

SEQ ID NO: 273 - DNA, 28 bp, S. Aureus
accccagcctgcctcctgtaacacttaa

SEQ ID NO: 274 - DNA, 28 bp, S. Aureus
actcttttctcggtatatattgtcagat

SEQ ID NO: 275 - DNA, 28 bp, S. Aureus
cggtatatattgtcagatatctctgggt

SEQ ID NO: 276 - DNA, 28 bp, S. Aureus
accccaactgtctgccagaaaccttgat

SEQ ID NO: 277 - DNA, 3258 bp, S. aureus
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagtaa

SEQ ID NO: 278 - DNA, 3030 bp, C. jejuni
atggccccaaagaagaagcggaaggtcggtgctcgcatactcgcttttgatattggaatttcatccataggatgggcattttcagaaaatgatgaacttaaagattgtggagtcaggattttcacaaaagtagagaatcccaaaacaggggaaagccttgctctcccaaggagactggcgcgatccgcaaggaaacgacttgctaggcgcaaagcaaggttgaatcatcttaaacatctcattgctaatgaatttaaactcaattatgaagattaccaaagttttgatgaatctttggctaaagcgtataaaggtagtctcatttccccatatgaactccgttttcgcgcattgaatgaacttctctctaaacaagattttgctcgtgtcattcttcacattgcaaaacgtcgcggttatgatgatattaagaattcagatgataaggaaaagggagcgattctcaaagctattaaacaaaatgaggagaaattggctaactatcaatctgtcggagaatatctctataaggaatatttccaaaagtttaaggaaaattccaaggaatttacaaatgtgcgaaataagaaggagtcctatgaaaggtgcattgctcaatcctttctcaaagacgaactcaaactcatctttaagaaacaaagggaatttgggtttagttttagtaagaagtttgaagaggaagtattgtcagtggctttctataaacgggctctcaaggacttttctcatctggtcggaaattgttctttctttacggatgaaaagcgggcaccgaagaattcaccactcgcgtttatgtttgtcgcactcactcgcattattaatctcctcaataaccttaagaatacagaaggaattctttatacaaaagatgatctcaatgcgctgcttaatgaagttttgaagaatggaactcttacttataaacaaacaaagaagttgcttgggttgtcagatgattatgaattcaaaggagagaaaggtacttattttatcgagtttaagaaatataaagagtttattaaagcactcggagaacataatctctcccaagacgaccttaatgaaattgcaaaagatattacactcattaaagatgaaataaaactgaagaaagcacttgcaaaatatgatctgaatcaaaatcaaatcgattcactttctaaattggagtttaaagaccatttgaatatttctttcaaagcacttaaattggtcacaccactcatgcttgaggggaagaaatacgatgaagcctgtaatgagcttaatttgaaagtcgctattaatgaagataagaaggattttcttccagcttttaatgaaacctattataaagatgaggttacgaatccggttgtcttgcgagcaattaaggaatataggaaagtactcaacgctttgctcaagaagtatggtaaagtacataaaattaatattgaacttgcccgcgaggtcggtaagaatcattcacaacgggctaaaattgaaaaggagcaaaatgaaaattataaagcgaagaaagacgcagaactcgagtgtgaaaagttgggcctcaaaattaattccaagaatatactcaagcttcggctgtttaaggaacaaaaggagttttgtgcatatagtggagagaaaatcaaaatctccgatcttcaagacgaaaagatgctggaaattgaccatatttatccatattctaggtcttttgatgatagttatatgaataaagtccttgtatttacaaaacaaaaccaggagaaacttaaccaaactccctttgaggcttttgggaatgattccgcaaaatggcaaaagattgaagtattggctaagaatctcccgaccaagaaacagaaacgaattttggataagaactataaagataaagagcagaagaattttaaagatagaaatctcaatgatactcgatacattgctcgccttgtcttgaattataccaaagactatttggactttctccccctctcagatgatgaaaataccaaattgaatgacactcaaaagggatcaaaagtccatgttgaggccaaaagtgggatgctcacttccgcactccgccatacgtggggattttccgcaaaagacaggaataatcacctgcatcatgctatagatgctgttataatagcatatgcaaataattccattgtcaaagccttttctgattttaagaaggaacaggaaagtaattctgcagaattgtatgctaagaagatttccgaactcgattataagaataaaagaaaattctttgaaccatttagtgggtttcggcaaaaggtcttggacaaaattgatgaaatatttgtcagcaaaccagaaaggaagaaaccatccggagcgcttcatgaagagacttttcggaaggaagaggaattttatcaaagctatggcggaaaagagggagttcttaaagcgttggagctcggtaaaatacggaaggtcaatggtaaaatagttaagaacggggatatgtttagggttgatatatttaaacataagaaaacaaataaattttatgctgttcccatttatactatggactttgcattgaaagtcttgccgaataaagcggtcgctaggtccaagaaaggagagattaaagactggatattgatggatgaaaactacgaattttgcttttccttgtataaagatagcctgattttgatacaaaccaaagatatgcaggaaccagaatttgtttattataatgcgtttacaagtagtactgtcagccttattgtctccaaacatgacaataaatttgaaaccctcagtaagaatcagaaaattttgtttaagaatgcgaatgagaaagaggttattgcaaaatccattggaattcaaaatttgaaggtattcgagaagtatattgtcagcgcgctcggagaggttactaaagctgaattccgccaacgcgaagatttcaagaaaaaaaggccggcggccacgaaaaaggccggccaggcaaaaaagaaaaagtga

SEQ ID NO: 279 - DNA, 6 bp, Artificial Sequence
  Synthetic
  misc_feature (1)..(2): n is a, c, g, or t
nngrrt

SEQ ID NO: 280 - DNA, 8 bp, Artificial Sequence
  Synthetic
  misc_feature (1)..(4): n is a, c, g, or t
nnnnryac

SEQ ID NO: 281 - RNA, 5 nt, Artificial Sequence
  Synthetic
uuuuu